(19) United States
(12) Patent Application Publication
(10) Pub. No.: US 2026/0007798 A1
(43) Pub. Date: Jan. 8, 2026

(54) Cotton Gauze Replacement for Temporary Use in an Oral Cavity

(71) Applicant: Queventive, LLC, Palm Beach, FL (US)

(72) Inventor: Joseph V Quevedo, Pittsford, NY (US)

(21) Appl. No.: 18/764,744

(22) Filed: Jul. 5, 2024

(51) Int. Cl. A61L 15/26; A61L 15/42

(52) U.S. Cl. CPC A61L 15/26; A61L 15/425

(57) ABSTRACT

An oral surgical retraction article comprises a polyester or polyether polyurethane foam sponge, or a combination thereof, that is sterilizable and has a porosity between not less than 45 pores per inch (ppi) and not more than 105 ppi and configured in size and shape for retracting a gingival flap during an oral surgery.

### REFERENCE TO RELATED APPLICATIONS

This patent application is related to U.S. patent application Ser. No. 18/762,776, filed Jul. 3, 2024.

This patent application is also related to U.S. patent application Ser. No. 18/157,069, filed Jan. 19, 2023, which claims priority to U.S. provisional patent application No. 63/300,881, filed Jan. 19, 2022.

This patent application is also related to U.S. patent application Ser. No. 17/913,019, filed Sep. 20, 2022, which is a 371 of PCT/US21/23544, filed Mar. 22, 2021, which claims the benefit of priority to U.S. provisional patent application Ser. No. 62/992,177, filed Mar. 20, 2020, and which is a continuation-in-part (CIP) of PCT/US21/21258, filed Mar. 5, 2021, which claims the benefit of priority to U.S. Ser. Nos. 62/985,731, filed Mar. 5, 2020 and 62/992,177, filed Mar. 20, 2020.

Each of these related patent applications is incorporated by reference.

### BACKGROUND

Dental implant surgeries and dental repair surgeries for, respectively, replacing and repairing a decaying tooth, a partially or entirely missing tooth, or an otherwise painful, unsightly, or unsuitable tooth are dental surgical options provided by dentists for resolving oral health issues for their patients. Cotton and gauze have been used as spacer materials to preserve the way for subsequently coupling abutments and crowns to installed implants following osseointegration and/or for facilitating reaccess to a coupling component or to a cavity or canal pathway in a follow-on checkup or procedure. However, cotton and gauze can become sticky and disheveled over time, especially when soaked with bodily fluids, and straggling cotton fibers can provide pathways for microbes. It is desired to have alternative spacer articles and materials that exhibit sufficient porosity and sterilizability, e.g., autoclavability, and can maintain their structural integrity, even when soaked in bodily fluids and subjected to oral vicissitudes, over extended periods of time.

Gingival retraction involves deflection of marginal gingiva away from a tooth. There exist multiple varieties of mechanical, chemo-mechanical, cordless and surgical retraction techniques. Retraction cords, chemical reagents, electrosurgery, laser tissue sculpting and hemostatic materials are often used when atraumatic displacement of gingival tissue is desired. Of these, gingival retraction cords are most commonly used, often in combination with chemical solutions, astringent gels, or hemostatic agents such as aluminum chloride which can cause gingival recession and can damage epithelial tissue and underlying connective tissues.

Gingival electrosurgery may be used for crevicular troughing but at a significant risk of causing long-term damage.

Retraction pastes have advantages such as comfort reported by patients, faster techniques, ease of use, no need for anesthesia, and reduced tissue trauma. Retraction pastes tend to perform less effectively at the deeper subgingival sites of deeper implants. Injectable materials can be used to form an expanding matrix to provide gingival retraction. As with retraction pastes, injectable matrices provide less effective retraction performance in procedures involving deeper implants.

FIG. 1A illustrates how cotton fibers are often left behind from uses of gauze during an oral surgery. The amount of such fibers that are left behind increase with the amount of use of gauze. FIG. 1B illustrates how cotton gauze leaves microscopic cotton fibers in the surgical field creating a foreign body reaction in the wound site and an increase in post-operative inflammation. It is desired to have a suitable replacement for cotton gauze in oral surgeries which leave no microscopic gauze fibers behind and instead remain intact during use. It is further desired to have a suitable replacement for cotton gauze which retains its shape and form throughout an oral surgery and does not stick to tissues at the wound site, thereby leaving mouth tissues intact without leaving any portion of the replacement material behind at the wound site when it is removed.

### BRIEF DESCRIPTION OF DRAWINGS

FIGS. 1A-1B illustrate cotton fibers that were left behind from use of gauze following an oral surgery.

FIGS. 1C-1D illustrate how a suitable gauze replacement material, e.g., polyether polyurethane or polyester polyurethane, leaves no gauze fibers behind and instead remains intact during an oral surgery.

FIGS. 2A-2C schematically illustrate a 3 mm thick, square foam qube in perspective, front and side views, respectively, in accordance with an example embodiment.

FIGS. 3A-3C schematically illustrate a 2 mm thick, square foam qube in perspective, front and side views, respectively, in accordance with an example embodiment.

FIGS. 4A-4C schematically illustrate a 10 mm thick, U-shaped foam qube, in perspective, front and side views, respectively, in accordance with an example embodiment.

FIGS. 5A-5C schematically illustrate a 12 mm thick, U-shaped foam qube, in perspective, front and side views, respectively, in accordance with an example embodiment.

FIGS. 6A-6C schematically illustrate a 15 mm thick, U-shaped foam qube, in perspective, front and side views, respectively, in accordance with an example embodiment.

FIGS. 7A-7C schematically illustrate an 8 mm thick, short rectangular foam qube, in perspective, front and side views, respectively, in accordance with an example embodiment.

FIGS. 8A-8C schematically illustrate a 10 mm thick, short rectangular foam qube, in perspective, front and side views, respectively, in accordance with an example embodiment.

FIGS. 9A-9C schematically illustrate an 8 mm thick, 2D foam mushroom qube, in perspective, front and side views, respectively, in accordance with an example embodiment.

FIGS. 10A-10C schematically illustrate a 10 mm thick, 2D foam mushroom qube, in perspective, front and side views, respectively, in accordance with an example embodiment.

FIGS. 11A-11C schematically illustrate a 10 mm thick, 2D foam football qube, in perspective, front and side views, respectively, in accordance with an example embodiment.

FIGS. 12A-12C schematically illustrate an 8 mm thick, 2D foam thimble qube, in perspective, front and side views, respectively, in accordance with an example embodiment.

FIGS. 13A-13C schematically illustrate a 10 mm thick, 2D foam thimble qube, in perspective, front and side views, respectively, in accordance with an example embodiment.

FIGS. 14A-14C schematically illustrate a 6 mm thick, long rectangular foam qube, in perspective, front and side views, respectively, in accordance with an example embodiment.

FIGS. 15A-15C schematically illustrate a 4 mm thick, 30 mm long rectangular foam qube, in perspective, front and side views, respectively, in accordance with an example embodiment.

FIGS. 15D-15F schematically illustrate a 10 mm thick, 20 mm long rectangular foam qube, in perspective, front and side views, respectively, in accordance with an example embodiment.

FIGS. 15G-15I schematically illustrate a 15 mm thick, 20 mm long rectangular foam qube, in perspective, front and side views, respectively, in accordance with an example embodiment.

FIGS. 16A-19 schematically illustrate polyether polyurethane grafting qubes of multiple sizes and shapes that are in accordance with example embodiments.

FIG. 16A schematically illustrates a quasi-football shaped grafting qube in accordance with an example embodiment.

FIG. 16B schematically illustrates a quasi-football shaped grafting qube that is scored in accordance with an example embodiment.

FIG. 17A schematically illustrates a large quasi-mushroom shaped grafting qube in accordance with an example embodiment.

FIG. 17B schematically illustrates a large quasi-mushroom-shaped grafting qube that is scored in accordance with an example embodiment.

FIG. 18A schematically illustrates a small quasi-mushroom shaped grafting qube in accordance with an example embodiment.

FIG. 18B schematically illustrates a small quasi-mushroom-shaped grafting qube that is scored in accordance with an example embodiment.

FIG. 19 schematically illustrates a quasi-truncated cone-shaped grafting qube that is scored in accordance with an example embodiment.

FIG. 20 schematically illustrates a felted rectangular polyester polyurethane dab qube that is scored in accordance with an example embodiment.

FIG. 21A illustrates another example embodiment of a U-shaped compression qube that is a polyether polyurethane foam material including a pore density of 50-100 ppi.

FIG. 21B illustrates another example embodiment of a U-shaped compression qube that is non-reticulated felted polyether polyurethane, and has a pore density of 40-90 ppi.

FIG. 22A illustrates a thick 2″×2″ polyurethane SG series sample that is reticulated, and is not felted, and has a pore density between 60-120 ppi, while in other example embodiments, SG series qubes may have up to 5× compression or double, triple, or quadruple felting, or up to quintuple felting, and may have pore densities that are between 60 ppi-600 ppi.

FIG. 22B illustrates a 2″×2″×2 mm polyester polyurethane SG series example embodiment that is reticulated and unfelted and has a density of about 100 ppi.

FIG. 23A illustrates a retraction qube in accordance with an example embodiment, that is reticulated, and triple felted, and had a starting pore density of about 100 ppi prior to the triple felting, and is rectangular on at least four of six sides, like a shipping container.

FIG. 23B illustrates a retraction qube in accordance with an example embodiment, that is non-reticulated, unfelted and had a starting pore density of around 77 ppi, and is a rectangular polyhedron and is rectangular on the four sides that are parallel, in one of its two dimensions, with the long axis of the rectangular polyhedron.

FIGS. 24A-24B illustrate front and side views of a tongue retractor qube in accordance with an example embodiment.

FIG. 25 illustrates an applicator qube in accordance with an example embodiment.

FIG. 26 illustrates a qube roll which may be similar to a cotton roll except a qube roll is not made of cotton, and is configured to provide saliva flow control during a dental surgical procedure. The qube roll may be cylindrical, tubular, and/or otherwise elongated rhombohedral, trapezoidal or rectangular or tubular and 3× felted or otherwise felted up to five times 5× or six times 6× compression.

FIGS. 27A-27H illustrate several ePTFE/PTFE or polytetrafluoroethylene qube example embodiments in side, bottom, cutaway and perspective views.

FIGS. 28A-28L illustrate steps in a dental implant surgical process in accordance with an example embodiment.

FIGS. 29A-29D show steps in multiple example oral surgical processes in accordance with an example embodiment.

FIGS. 29E-29Y show another process in which a retraction qube may contribute to a positive result in an oral surgery.

FIGS. 30A-30K show steps in an oral surgical implant placement process in accordance with an example embodiment.

FIGS. 31A-31L illustrate steps in an oral surgical process involving a further use of a retraction qube in accordance with an example embodiment.

FIG. 32A-32V illustrate an example oral surgical process involving an endo qube.

FIGS. 33A-33F illustrate an example process that involves use of one or more endo qubes.

FIGS. 34A-34U illustrate a dental implant process following a tooth extraction that involves use of an implant qube.

FIGS. 35A-35J illustrate an oral surgical process involving use of an anterior implant qube.

FIGS. 36A-36P illustrate an oral surgical process involving use of both an exo qube and a grafting qube.

FIGS. 37A-37E illustrate another oral surgical process involving use of a graft qube.

FIGS. 38A-38F-illustrate qubes for use with oral surgical processes involving anterior teeth, premolar teeth, and molar teeth.

FIGS. 39A-39N illustrate retraction tools in accordance with example embodiments.

FIGS. 39O-39P illustrate SG series qubes as substitutes for cotton gauze in accordance with example embodiments.

FIGS. 40A-40G illustrate oral surgical process steps involving use of qubes for enhancing hemostasis, wound cleaning, wound healing, excess saliva and blood absorption and suctioning.

FIGS. 41A-41Q illustrate one or more dental implant processes involving use of one or more retraction qubes.

FIGS. 42A-42G illustrate one or more tooth extraction and/or dental implant processes involving use a compression qubes or C series qubes or C qubes in accordance with example embodiments.

FIG. 43A-43L illustrate one or more oral surgical processes involving use of endo qubes in accordance with example embodiments.

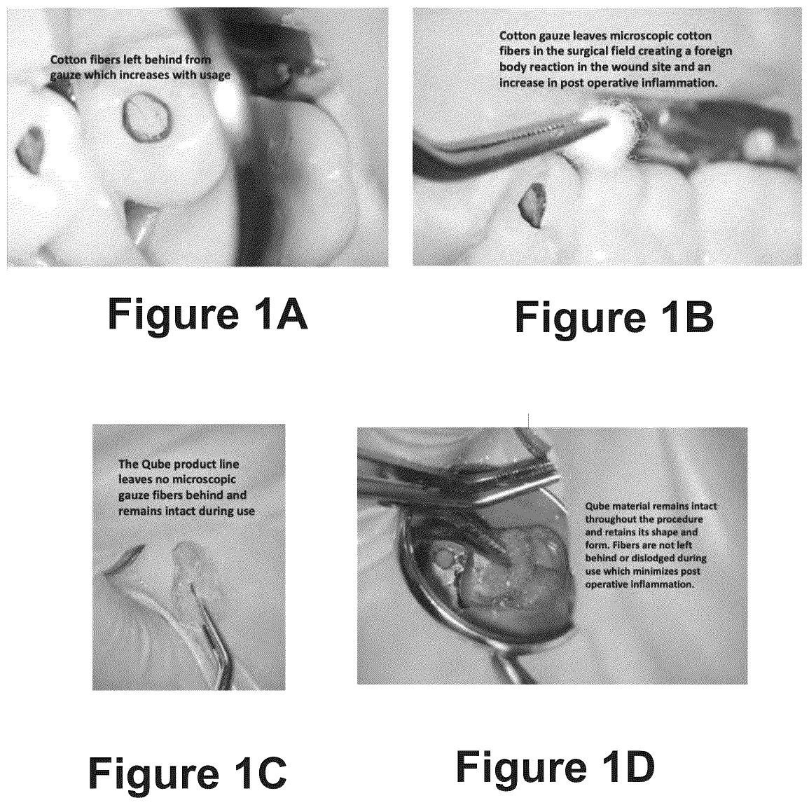

### DETAILED DESCRIPTIONS OF EXAMPLE EMBODIMENTS

A dental surgical retraction article is provided that may include a polymeric foam sponge that may be sterilized, e.g., by steam autoclave sterilization, gamma or electron beam radiation, Ethylene Oxide (EtO) sterilization, hydrogen peroxide or formaldehyde gas exposure, or heating or freezing or applying another sterilization technique that may be suited to a particular polymeric foam material. The dental article may have a porosity that is not less than a porosity of polyurethane. The dental article may be configured in size and shape for retracting a gingival flap during an oral surgery.

The polymeric foam sponge may include polyurethane foam, such as polyester polyurethane foam or polyether polyurethane foam. The polyurethane foam may exhibit a pore density between 30 ppi to 120 ppi, 45 ppi to 105 ppi, 60 ppi to 90 ppi, 70 ppi to 80 ppi, or another specific range that is optimal to a specific material and use.

The polymeric foam sponge may exhibit an elongated shape. The polymeric foam sponge may include a cylindrical, ellipsoidal, tubular, wedge, prism, ovoid, triovoid, egg or pear shape, or combinations thereof.

The polymeric foam sponge may include polyurethane, polytetrafluoroethylene (PTFE), ePTFE, polyolefin, polyamide-imide, polymethylpentene (PMP), polyoxymethylene (POM), polyaryletherketone (PAEK), polyetheretherketone (PEEK), non-reticulated or wholly or partially reticulated polyether type polyurethane, polyethyl polyurethane, thermoplastic foam, reactive resin foam, polyurethane foam, reaction injection molding plastic foam, flexible foam, thermoplastic polyurethane, mica-particulated polyurethane, resin-particulated polyurethane, resin-blended polyurethane, porous polyurethane, polyester polyurethane, polyether polyurethane, or polyurethane blend, or combinations thereof. The polymeric foam sponge may be felted or non-felted, and reticulated, wholly or partially, or non-reticulated. The amount of felting can be unfelted, 1× felted, or 2×, 3×, 4×, or 5× felted.

The polymeric foam sponge may include polyurethane blended with one or more additives for enhancing one or more characteristic material attributes. The one or more additives may include silicon oil, silicone surfactant, polyester, polyether, polyethyl, or molybdenum.

The one or more additives may include ethylene glycol, 1,4-butanediol (1,4-BDO or BDO), 1,6-hexanediol, cyclohexane dimethanol or hydroquinone bis(2-hydroxyethyl) ether (HQEE), or combinations thereof.

The one or more additives may include one or more difunctional, trifunctional or tetrafunctional Hydroxyl compounds or one or more difunctional amine compounds, or combinations thereof.

The one or more additives may include one or more difunctional hydroxyl compounds including Ethylene glycol, Diethylene glycol, Triethylene glycol, Tetraethylene glycol, Propylene glycol, Dipropylene glycol, Tripropylene glycol, 1,3-Propanediol, 1,3-Butanediol, 1,4-Butanediol, Neopentyl glycol, 1,6-Hexanediol, 1,4-Cyclohexanedimethanol, HQEE, Ethanolamine, Diethanolamine, Methyldiethanolamine, or Phenyldiethanolamine, or combinations thereof.

The one or more additives may include one or more trifunctional hydroxyl compounds including Glycerol, Trimethylolpropane, 1,2,6-Hexanetriol, or Triethanolamine, or combinations thereof.

The one or more additives may include one or more tetrafunctional hydroxyl compounds including Pentaerythritol, N,N,N′,N′-Tetrakis, (2-hydroxypropyl), or ethylenediamine, or combinations thereof.

The one or more additives may include one or more difunctional amine compounds including Diethyltoluenediamine or Dimethylthiotoluenediamine, or both.

A dental surgical retraction article is also provided that includes a sustainable green polyhydroxurethane foam sponge formed by combining polyamines and cyclic carbonates with polyols prepared from vegetable oils, dimer fatty acids, or fatty acids, or combinations thereof.

A method of manufacturing a dental surgical retraction article is also provided. The method may involve combining one or more aromatic isocyanates, or diisocyanates, or aliphatic or cycloaliphatic isocyanates with one or more polyols including at least one polyether or polyester polyol that has a molecular weight of at least 2000.

The one or more polyols may include polycarbonate, polycaprolactone, polybutadiene, polysulfide, castor oil, soybean oil, cotton seed oil, neem seed oil, vegetable oil, dipropylene glycol, glycerine, or a sorbitol/water solution, or combinations thereof.

The method may also include chemically grafting dispersed styrene-acrylonitrile, acrylonitrile, or polyurea (PHD) polymer solids to a polyether or polyester backbone.

The one or more isocyanates may include 1,6-hexamethylene diisocyanate (HDI), 1-isocyanato-3-isocyanatomethyl-3,5,5-trimethyl-cyclohexane, isophorone diisocyanate (IPDI), or 4,4-diisocyanato dicyclohexylmethane (H12MDI or hydrogenated MDI), or combinations thereof.

A dental implant surgical spacer article is also provided. This dental article may include a polymeric foam sponge or ePTFE that is sterilizeable and has a porosity not less than a porosity of polyurethane. The dental article may be configured in size and shape to preserve a volume above a dental implant for coupling an abutment to the dental implant during an osseointegration period.

The polymeric sponge may include a base end opposite a tapered end.

The polymeric sponge may include a tapered end to base end weight density ratio of at least 2:1.

The polymeric sponge may exhibit a conic or truncated conic shape.

The polymeric sponge may include a pyramid or truncated pyramid shape.

An endodontic spacer article may include a polymeric foam sponge that may be configured to temporarily preserve a prepared tooth cavity volume until filling material is ready for filling the cavity volume with permanent filling material.

The polymeric sponge may include an absorbed, adhered or trapped medicinal dosage, or combinations thereof.

The polymeric sponge may include a base end opposite a tapered end.

The polymeric sponge may include a tapered end to base end weight density ratio of at least 2:1.

The polymeric sponge may include a conic or truncated conic shape.

The polymeric sponge may exhibit a pyramid or truncated pyramid shape.

A polymeric foam sponge may be configured to protect sensitive or vulnerable mouth tissue from surgical equipment and ambient exposure during an oral surgery.

A dental surgical retraction method is also provided that may include placing a polymeric foam sponge at a gingival incision location to retract the gingival flap during an oral surgery.

A dental surgical spacer method is also provided. The method may include placing a polymeric foam sponge in a space next to an embedded dental implant to preserve a spacing for coupling an abutment to the dental implant after an osseointegration period.

A dental surgical protection method is also provided. The method may include placing a polymeric foam sponge against sensitive or vulnerable mouth tissue as protection from surgical equipment impacts and ambient exposure during an oral surgery.

A dental article is also provided. The dental article may include a polymeric foam sponge that is sterilizeable and has a porosity not less than a porosity of polyurethane and is configured for insertion into a bone socket recess to nonadhesively contact and compress loose graft material contained therein.

The polymeric foam sponge may include polyurethane or polyurethane blend.

The polymeric foam sponge may include a porosity not less than a porosity of polyurethane.

The polymeric foam sponge may be further configured for maintaining a volume density integrity of compressed graft material when removing bodily fluids from the bone socket recess by suctioning said fluids through the polymeric foam sponge.

A guided tissue regeneration membrane may be configured to be disposed between graft material and a polymeric foam sponge during compression of the graft material by applying contact pressure nonadhesively to the sponge.

The membrane may be configured to remain over the graft material within the socket graft recess during an osseointegration period.

A dental bone socket grafting method is also provided. A bone socket recess defined within a patient's jawbone is prepared. After the preparing of the bone socket recess, the bone socket recess is filled with loose graft material. The loose graft material may be compressed within the bone socket recess by inserting a nonadhesive polymeric foam sponge into contact with the loose graft material therein and applying pressure to the sponge. The nonadhesive polymeric foam sponge may exhibit sterilizable and may have a porosity which is not less than a porosity of polyurethane.

The preparing of a bone socket recess may involve shape cutting or drilling into a tooth, or through gum tissue, or into some bone tissue, or combinations thereof.

The preparing of a bone socket recess may involve removing one or more of a decayed tooth, decayed tissue, excess tissue, microbial organic material, or inorganic debris, or combinations thereof.

The method may include suctioning fluid from the bone socket recess through the sponge.

A tooth extraction process may include using an U-shaped and/or H-shaped compression qube.

A dental surgical process may include using SG series qubes and/or a medicated MS series qube.

A dental hygiene process may include using a Qube roll, cotton roll, or applicator qube, or combinations thereof. One or more smooth and/or ribbed qubes may be used. A dental surgical process may include using a tongue retraction qube.

The text descriptions that follow generally refer to specific additional example embodiments that are illustrated schematically and/or in photographs, tables and/or other graphics referred to in U.S. patent application Ser. No. 17/913,019, which is by the same Inventor and Applicant, and which is hereby incorporated by reference. Many components and parameters embody concepts that are described herein with reference to multiple working examples.

A qube may have a size, shape and/or color that has been selected in accordance with a specific use and function during an oral surgery in accordance with an example embodiment.

Qubes may have different sizes and shapes selected, and optionally cut from, a larger qube. Each individual qube may be custom configured for a specific intended use during an oral surgery in accordance with example embodiments. Several qubes of each of several types, shapes, sizes, and compositions are illustrated and/or described in example embodiments herein.

In some example embodiments, a qube may relate to an article for application to human and/or animal teeth and human and/or animal dental implants as a medicated and non-medicated space maintainer and/or retraction medium (referred to herein as a QUBE, a Qube, or a qube). A Qube may include, in an example embodiment, a synthetic sponge-like material and/or polymeric foam with a 1) specific porosity size. 2) which is sterilizable, e.g., by any one or a combination of a number of techniques, such as using ethylene oxide or hydrogen peroxide or formaldehyde gas exposure, or electron beam or gamma ray beam irradiation, or autoclaving or heat or steam exposure, or freezing, or exposure to sterilizing liquids, or combinations thereof 3) which can optionally be colored 4) which may be used as a vehicle to carry a medicament 1.2% Chlorohexidine, or 5) which can be used as a vehicle to carry a medicament Calcium hydroxide Ca(OH), or 6) which can be used as a vehicle to carry a medicament Povodine-Iodine solution, or 7) which can be used as a vehicle to carry a medicament 2% Iodine Potassium Iodide, or 8) which can be used as a vehicle to carry a Sterile saline, or combinations thereof. A Qube may be configured to be applied as an interappointment dressing for endodontically treated teeth in an access cavity to serve as a barrier from microbial invasion of a canal space as well as a mechanism to prevent damage to surrounding tooth structure when a dentist re-accesses the tooth for permanent restoration. A Qube may also be configured to be used as a barrier from microbial invasion within an internal aspect of a coronal access of a screw retained dental implant.

In another example embodiment, a Qube can be configured to be used as a retraction medium for gingival flaps during dental surgery. The Qube can be contoured in specific shapes. In another example embodiment, a Qube can be impregnated with barium sulfate so it can be visible radiographically. A Qube can also be impregnated with other radio opaque agents or a scannable receiver agent that can be imbedded in the retraction foam and can be detected with a receiver instrument if inadvertently left inside the wound site accidentally.

In another example embodiment, a Qube can be configured to be inserted and compacted against gingival soft tissue to allow for atraumatic retraction. In this embodiment, a Qube is used for atraumatically stretching soft tissue elastic fibers during retraction, which helps with closure of a wound as it reduces tension of the soft tissue when suturing. The atraumatic retraction also compresses against blood vessels to allow for a hemostasis affect, thereby allowing for better visibility and control during the procedure at the surgical site, and reducing a risk of surgical errors being made due to an inability to sufficiently clearly visualize the surgical site. This affect also prevents the need for chemical or physical agents that can be more harmful to the surgical site when used to control bleeding. The physical compression and retraction of the tissue with the retraction Qube avoids this and allows for better wound healing, less postoperative pain, and inflammation of the wound site.

In another example embodiment, a retraction Qube may be configured to create homeostasis from compression of blood vessels, minimizing trauma. The Qube in this example may be configured and positioned to stretch elastic fibers of a flap atraumatically for better wound closure and by applying less tension from sutures on the wound for improved healing and NS Pin reduction. The same qube can serve to improve visibility of a wound site, e.g., by absorbing bodily fluids.

Qubes may be used in various roles during oral surgical procedures, including as dental implant spacer qubes, and as grafting qubes, and as exo-socket Qubes, and as tapered endoQubes. Each of these different qube types, including implant cubes, grafting qubes, exo-socket qubes and endo-socket qubes may be configured in medicated or non-medicated form in accordance with example embodiments.

A tapered endo qubes may be provided in accordance with example embodiments. The tapered shape of a wedge shaped qube renders it advantageous for insertion into a dental implant space reserved for coupling with an abutment component or an abutment space reserved for coupling with a crown component for a duration of an osseointegration of the implant. Such a wedge may have four long sides and a square or rectangular base. Two long sides may be parallel, tapered and/or triangularly-shaped and the other two sides may be rectangular and form an acute angle at the tapered end.

One or both of the rectangular long sides may also be tapered or be triangularly-shaped, and a pyramidal qube or tetrahedral qube or truncated pyramid or truncated tetrahedron or cone-shaped, four or five sided pyramid, or pentagonal cone, pentagonal pyramid, truncated cone, half ellipsoid or partial ellipsoid or truncated ellipsoid. One or more of the long sides of a regular rectangular box, cube or polyhedron may be tapered or compressed spatially at one end.

An implant qube may be made of ePTFE or PTFE foam, or another polymeric material, foam material or sponge material, or combinations thereof that is more densely-weighted at a tapered end, which may taper to a point or may round off or may be truncated such that a plane at a tapered end may be parallel to a base plane of greater area of a truncated implant qube, which may have small and large diameter circular end planes, or an elongated end plane quadrilateral having at least one short dimension which may taper to a point in one or both cross-plane dimensions.

Different qubes may have different physical, chemical or biological properties, different functions, different uses, different roles to play within oral surgical applications, different compositions (polymer units or polymer side chains, molecular component monomers or side chains, monomer units or monomer side groups, different sizes (millimeters to centimeters) and shapes (spheres, ellipsoids, cubes, polyhedrons with four to twenty-four sides, wedges, pyramids, tetrahedrons, tapered polyhedrons, truncated polyhedrons, ovoid) and being grouped according to anticipated, intended or scheduled uses, functions, or specific applications among multiple example oral surgical applications in accordance with example embodiments.

Other qube types may include qubes having different colors or color distributions or different weights, or different overall weight densities (10 kg/m3, 15 kg/m3, 20 kg/m3, 25 kg/m3, 30 kg/m3, 35 kg/m3, 40 kg/m3), or different weight or weight density distributions (20&30 kg/m3, or different porosities, or different applicability to sterilization methods including but not limited to autoclavabilities (thermal: 250 F-300 F, 225 F-325 F, 200 F-275 F, 200 F-280 F, 270 F-280 F, 275 F-300 F, 275 F-325 F; pressure: 20 psi-30 psi, 25-35 psi, 25-30 psi, 23-28 psi, 24-27 psi, 28-36 psi, 24-38 psi)-indentation force deflection (IFD) capabilities, cell openness, cell densities (15-16 cells/cm, 10-20 cells/cm, medicament chemistry (calcium hydroxide CaOH, barium sulfide BaS, titanium dioxide TiO2, silver nitride Ag3N, silver nitrate AgNO3, silver ion Ag+, silver ion Ag−, 1.2% chlorohexidine, povodine-iodine 2% iodine potassium iodide, sterile saline, as an embedded sensor within a qube product providing an ability to detect it with a receiver device, or medicament biology (e.g., bacteriocidal), and/or different medicament combinational process types (e.g., soaking, coating, encapsulating, embedding, integrating, release rate).

Moreover, in certain example embodiments, use of a qube during an oral surgery or during a step or subset of steps of an oral surgery, e.g., a dental implant surgery, a tooth or jawbone grafting surgery, or another oral surgery involving one or more retraction uses of one or more cubes. In example embodiments, a dental impression may be made, formed, generated or located such as to make a dental impression for molding a synthetic tooth, a grown organic tooth or a tooth graft or set of teeth to replace a tooth or teeth that may have become decayed or that may be colliding with another tooth or gum, cheek, tongue or lip area causing pain or that may be rooted unevenly within an upper or lower jaw in the front or back of the mouth or may have fallen out such that a synthetic replacement tooth or a grown organic replacement dental implant or similar oral constituent may be desired to take its place.

Example embodiments are provided herein that may involve one or more oral surgical steps, sequences of two or more steps, subsets of multiple steps or several steps, or complete oral surgical processes that involve use of a qube for retraction, maintaining space above or within a dental implant, abutment or crown, or providing temporary structural integrity support for a tooth, gum, dentin, pulp, root, enamel, bone-cementum, crown or combinations or component parts thereof, or for catching, filtering, redirecting, accumulating, or stabilizing or controlling flow rate, area coverage or contained volume density of bodily fluids, saliva, blood, mucous, water, partly digested food or dislodged food fragments or combinations or evolving quantities or components thereof during an oral surgery.

Example embodiments may advantageously further involve reduced pain, reduced swelling, and reduced tearing, scratching, slicing, stabbing or poking by sharp edges or jagged components of dental instruments, and reduced time to heal and enhanced effectiveness by placement and use of one or more qubes for protecting, cushioning, deflecting, bandaging, or covering one or more exposed, wounded, inflamed or otherwise sensitive areas within a patient's mouth during an oral surgery.

Example embodiments of dental processes, both surgical and non-surgical, advantageously include sequences of steps involving use of one or more qubes for retraction, maintaining space, cushioning, absorbing, softening, providing flexibility, strength without rigidity, and cohesiveness. After any of a wide variety of oral surgical steps, and in various orders and sequences of oral surgical steps, use of qubes throughout the surgical processes characteristically maintains an availability of choices of next steps, when to stop, how to provide a first dental care process and transition to a different oral state prepared to provide a second dental care process, while continuously, discretely, periodically and/or increasingly having an ability to return, and/or returning, suturing or positioning or orienting tissue to an original position or orientation due to no distortion or damage being caused by this retraction method involving use of a qube rather than a conventional retraction cord or other conventional retraction device or component.

Other uses of qubes include performing a spacer or space-maintaining function during an oral surgical procedure that includes two or more subsets of an overall surgery or of a complete procedure, such as between coupling a dental implant at a grafted or un-grafted jawbone socket site which has become decoupled from a tooth suddenly or gradually over time, or a jawbone site that is at risk of becoming decayed unless a rotting tooth is extracted or repaired.

The two or more subsets of sequential oral surgical steps, processes, actions or modifications may, in one example embodiment, be spaced apart in time. In an example embodiment, a time delay advantageously allows for sufficient osseointegration of a bone graft within a jawbone socket, or socket graft, for example, prior to a dental implant procedure. Such a dental implant procedure may itself follow a sudden, unexpected tooth loss collision event or a long and steady incremental tooth decay process, or an ordinary tooth extraction, or a drawn-out, crumbling tooth disintegration lasting perhaps years or another tooth and/or jawbone volume reducing event.

The two or more surgical process subsets may, in another example embodiment, be spaced apart in time in order to allow sufficient osseointegration of a dental implant inserted within a jawbone socket at a depth below a gingival margin anywhere in a range between a shallow implant coupling location through an average implant depth location to a deep implant coupling location that may be significantly below a gingival margin. In this example embodiment, a second surgical process subset may involve coupling within a dental implant component for maintaining a space for attaching an abutment after sufficient osseointegration of the implant has occurred over the passage of time.

Retraction Qubes allow gingival flaps to be retracted for better visibility, to expose the implant atraumatically, and to allow the operator to access and place a healing abutment for the implant and then retrieval of, the retraction Qube, leaves a viable tension free flap that can be closed with primary closure with very low tension on the wound edges because of the modest fiber stretching from the retraction qube that was in place prior.

An access cavity within a crown may be filled with an Implant Qube to maintain the space for a screw or cement retained abutment that may be inserted after primary healing (3-4 months) is completed.

One or more qubes may be used to retract a rubber dam. A qube may be used to retract soft tissue as well. A qube may be used to protect cheek tissue, tongue tissue, lip tissue, gum tissue, tonsil tissue, and/or tissue at the roof of the mouth or under the tongue from a surgical drill or other surgical instruments. A qube may protect tissues of the mouth from encountering tooth or implant fragments which may have sharp or jagged edges by adhering, blocking or deflecting such items.

One or more Retraction Qubes may be used to both retract the tongue and retract the thin lingual tissue so a bone sequestrumectomy could be completed. A qube may also be configured and placed to protect surrounding soft tissues from trauma.

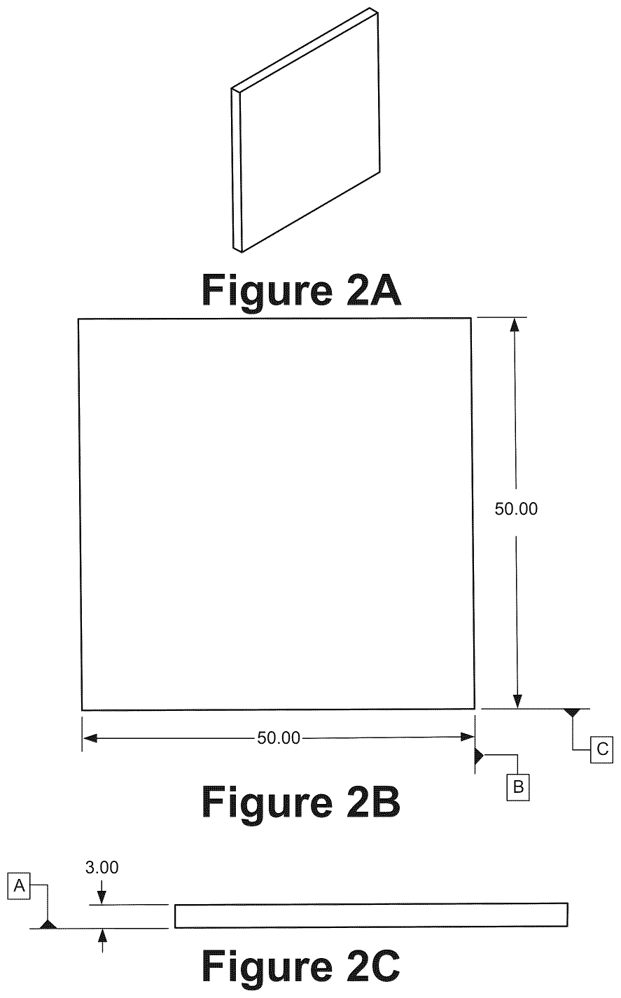

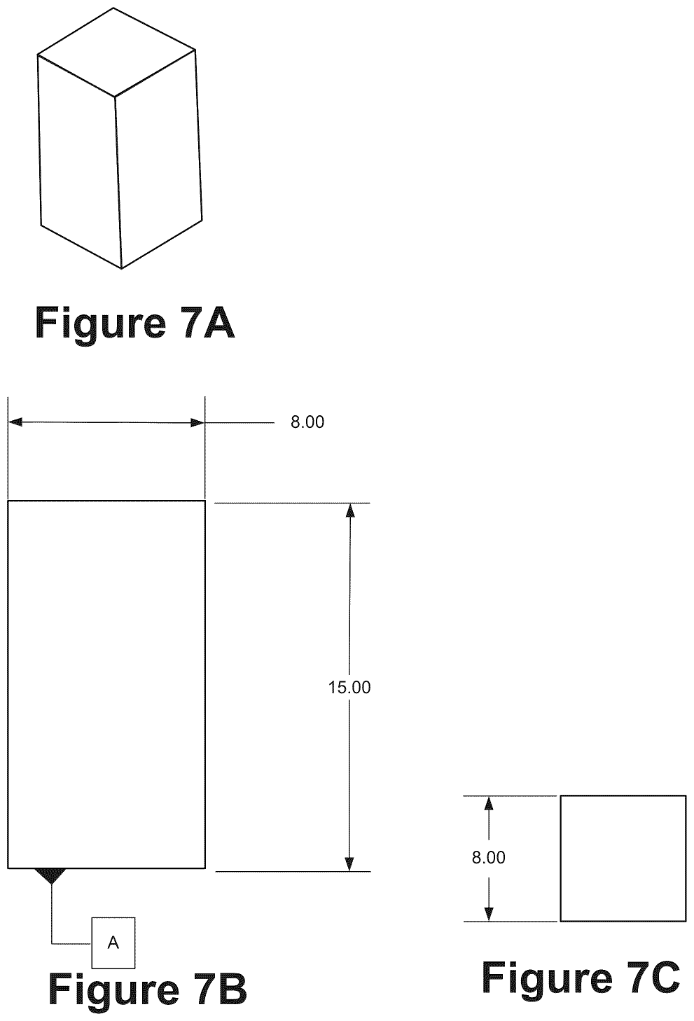

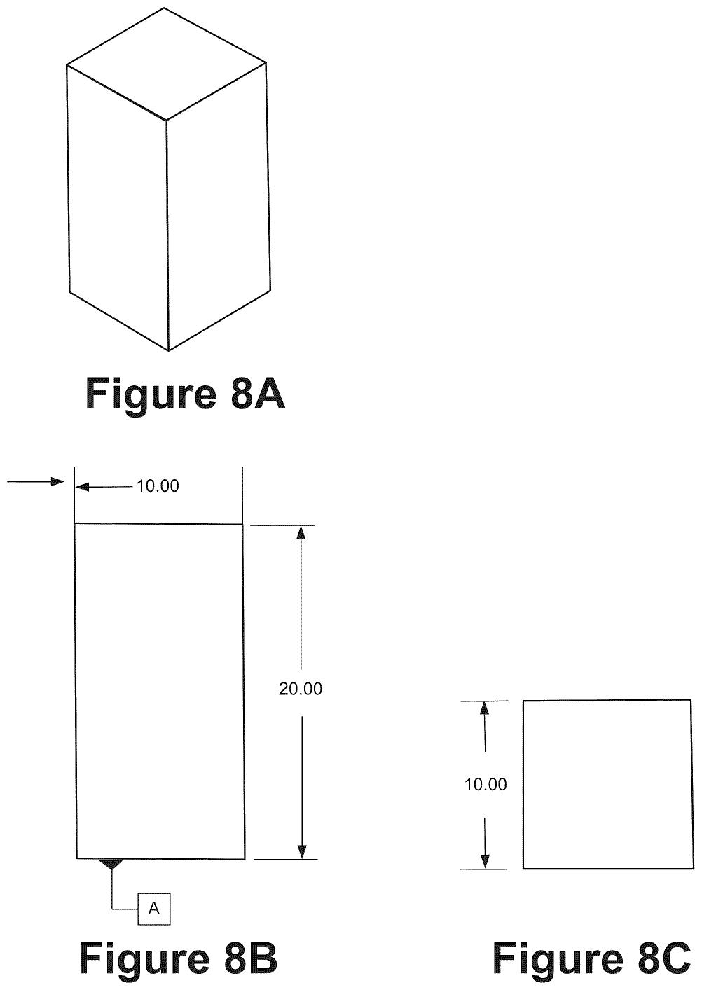

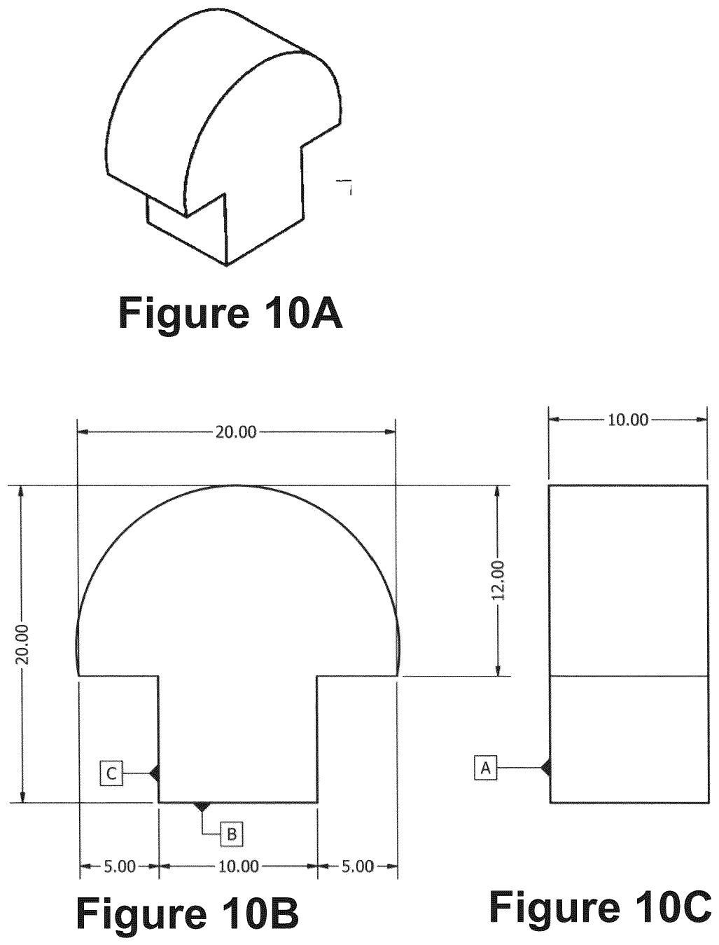

Example embodiments include the following five specific Qube embodiments that are illustrated schematically at FIGS. 2A-15I and described below with reference thereto. The dimensional ratios, overall sizes and volumes, surface areas, thicknesses and curvatures, and lengths of sides are illustrative, and additional embodiments may vary with regard to each of these and other parameters described herein.

A pore density of a qube may be selected in accordance with an application to which a qube is to be advantageously used. In example embodiments, a qube may also be selected that is advantageously either felted or unfelted, and in felted embodiments, a qube may undergo double felting or triple felting, or a qube may be compressed 2×, 3×, 4×, or 5×, for example, or between 2× and 4× compression, or between double felting and quadruple felting, or between 2.5× and 3.5×, or between 2.5:1 and 3.5:1, or in another range that may be centered around approximately 3× compression, triple felting or 3:1. In a specific example embodiment, a 3:1 felted, 30-110 ppi, e.g., 60-90 ppi or 70-90 ppi, or unfelted 50 ppi-120 ppi, polyester polyurethane, polyether polyurethane and/or polytetrafluoroethylene (PTFE) or ePTFE expanded foam qube may be used as a gauze replacement in various applications to which cotton gauze is conventionally used.

In different applications, pore densities of example embodiments of qubes may include between not less than 30-50 ppi) and perhaps not greater than 120-125 ppi. Beneath 30-50 ppi, e.g., around 40 ppi, structural integrity may become an issue, while above 120-125 ppi, e.g., even around 105-110 ppi, absorption and fluid transmission using suction can begin to become too low to be useful for this application and certain other applications. For example, when serving as a suction filter to draw excess blood and saliva through as part of a dental procedure, qubes between not less than 30-50 ppi, e.g., between 45 ppi and 75 ppi or perhaps more if desired for other reasons, or between 45 ppi and 70 ppi, or between 50 ppi and 65 ppi or between 55 ppi and 60 ppi may be selected. A cylindrical 3D mushroom shaped qube or a 2D mushroom shaped qube may be used for compacting graft material at a tooth extraction site and/or for suctioning blood and/or saliva therethrough without disturbing the grafting material and in certain embodiments serving to compress the grafting material by contact and application of pressure. Multiple applications, e.g., two or three or four or five, of supplying loose grafting material at a tooth extraction site may be performed sequentially each involving compacting the grafting material by contacting and applying pressure to the graft material with a mushroom shaped qube or a quasi-mushroom shaped qube having a mushroom shape in fewer than all three dimensions. The suction can be used to draw excess blood and saliva from the field without disturbing the graft beneath and retaining the majority of bone graft material.

A U-shaped qube may be installed at a tooth extraction site for allowing a patient to bite down comfortably to stop the bleeding and/or to compress loose grafting material without the U-shaped qube adhering to the grafting material and pulling it out when the U-shaped qube is removed. In each case, excess blood and/or saliva may be drawn or siphoned or suctioned through a qube which may contact graft material without pulling the graft material out of the socket nor otherwise loosening a compressed grafting pile when the qube is withdrawn or removed. Examples of U shaped qube embodiments may include felted polyester polyurethane or polyether polyurethane that are between 2:1 and 4:1 or 5:1 felted, e.g., approximately 3:1 felted, and between not less than 30-50 ppi and not more than 120-125 ppi, or between 50 ppi and 100 ppi, or between 60 ppi and 90 ppi, or between 70 ppi and 80 ppi, or between 70 ppi and 75 ppi, or approximately 72 ppi.

Another example embodiment may include a polyether polyurethane foam qube between, e.g., 30 ppi and 90 ppi, or between 40 ppi and 90 ppi, or between 50 ppi and 90 ppi, or between 60-65 ppi and 90 ppi, that may be selected for grafting applications when enhanced durability is desired.

In another example embodiment, a polyester polyurethane qube may have a pore density in a range between not less than 45-50 ppi and not more than 70-75 ppi or between 55 ppi and 65 ppi or between 57.5 ppi and 62.5 ppi or between 59 ppi and 61 ppi or within another range around approximately 60 ppi.

In the context of use of qubes to apply compression at a wound site, a felted qube including between 70 ppi and 105 ppi or between 80 ppi and 100 ppi or between 85 ppi and 95 ppi or another range around 90 ppi may be selected. In the context of use of qubes as wound disinfection tools, e.g., at a tooth extraction site, example embodiments may include felted qubes that include pore densities between, e.g., not less than 30 ppi and 120 ppi, or 40 ppi and 110 ppi, or 50 ppi and 100 ppi, or 60 ppi and 90 ppi, or 80 ppi and 90 ppi.

As a retraction instrument, an example embodiment may include a rectangular qube with a pore density between 30, 40 or 50 ppi and 85 ppi, or between 60 ppi and 80 ppi, or between 65 ppi and 75 ppi or another range around approximately 70 ppi. In other example embodiments, retraction qubes having higher or lower pore densities between 40 ppi and 125 ppi, felted or unfelted, may be selected.

### SG Series Qube

An SG series qube may be used in an example embodiment primarily for applications where traditional cotton gauze is conventionally used in the oral cavity or externally on the skin surface. An example SG qube may have a square shape, e.g., 50 mm×50 mm sheet, and a 2 mm-3 mm thickness. These can be advantageously used for applications where traditional 2″×2″ or 4″×4″ gauze pads are conventionally used such as for absorbing blood and saliva from wound sites and applying to tooth extraction sockets to initially compress the surface of the wound for initial clotting.

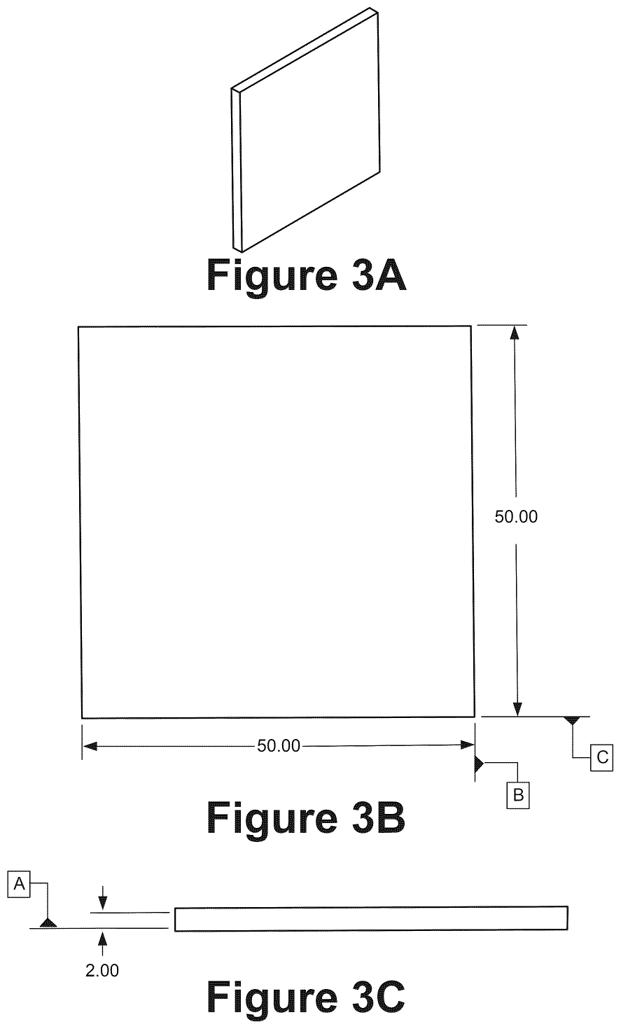

FIGS. 2A-2C and 3A-3C schematically illustrate perspective, top and side views of example embodiments of SG series qubes. An SG qube does not adhere to the surfaces of wound sites, especially after compressing the wound for initial hemostasis, so that when an SG qube is removed, the SG qube does not pull away the initial fibrin clot that has formed. In addition, SG qubes absorb fluids including blood and saliva and can be easily wrung out to reuse immediately. The pore size and reticulated nature of the foam material of a SG qube also allows for the material to be suctioned directly on its surface to draw out blood or saliva from the site, while the SG qube remains in place to protect the wound. The material can be rolled, folded or used in multiple layers for the procedure at hand.

In further example embodiments, SG qubes can be used to apply medicaments such as betadine or chlorohexidine to the surface of intra oral or extraoral wounds in a non-stick fashion or application of isopropyl alcohol to external surface sites receiving anesthetic. The SG qube also does not leave small cotton fibers behind in the wound the way traditional cotton gauze can do which can cause inflammation as the site heals over a prolonged healing time.

An SG series qube in accordance with an example embodiment may be configured for and/or used for applications in place of traditional cotton gauze. SG qubes may be square shaped, e.g., 50 mm×50 mm or 100 mm×100 mm, sheets that may be 1-10 mm thick, including 2 mm or 3 mm in thickness as in the examples of FIGS. 2A-2C. and/or FIGS. 3A-3C, which may be the same objects in many respects, respectively, while differing only in size and/or perhaps modestly in pore density, pore size, cell size, amount of compression or starting material composition. Other examples of SG Qubes include 25 mm×25 mm or smaller, 75 mm×75 mm, and 125 mm×125 mm or larger. A qube can be formed in example embodiments as most any shape, diameter or thickness, depending on the application.

A thick 2″×2″ polyurethane SG-Qube example embodiment that is reticulated, is not felted, and has a pore density between 60-120 ppi. SG Qubes in some example embodiments can be felted 2×-5× and have a pore density between 60 ppi-500 ppi. A 2″×2″×2 mm polyester polyurethane SG qube example embodiment may be reticulated and unfelted and have a density of about 100 ppi.

An SG series qube may be advantageously used for applications where traditional 2″×2″, 3″×3″ or 4″×4″, for example, cotton gauze pads are conventionally used such as for absorbing blood and saliva from wound sites in the oral cavity or externally applied to skin wounds. Lots of teeth are extracted and the SG product can easily be substituted for cotton gauze and applied to extraction sockets to initially compress the surface of the wound for initial clotting. Doctors typically fold the flat cotton gauze to their desired shape and size and then the patient bites down on the wad of gauze. This example is for typically a short time usage during a dental procedure. The SG qube does not adhere to the surface of wound sites like cotton gauze often tends to do, especially after compressing the wound for initial hemostasis. So when a qube is removed from a wound site, unlike cotton gauze, a qube does not tend to pull away the initial fibrin clot that forms. In addition, qubes tend to absorb fluids including blood and saliva and can be easily wrung out to reuse immediately. The pore size and reticulated nature of the foam material of an example qube allows for material to be suctioned during a dental procedure to draw out blood or saliva from an oral surgical site, while the super gauze qube may be left in place to protect the wound. The material of an example qube can be rolled, folded or used in multiple layers depending on the procedure at hand.

### C Series Compression Qube

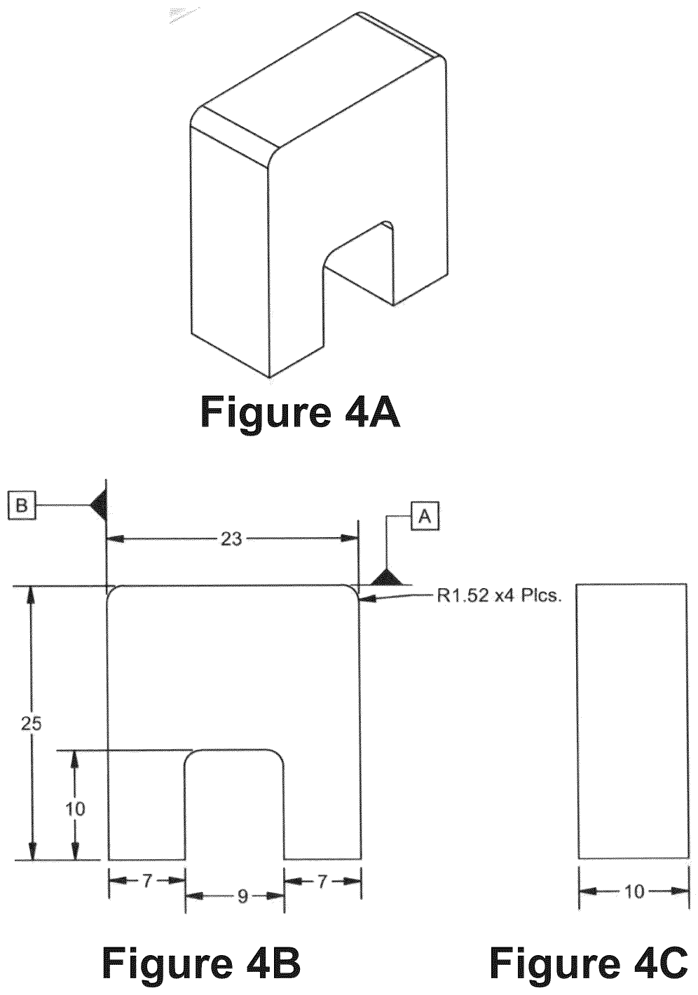

A compression qube or C-series qube or simply a C qube may be used to decompress an oral surgical wound site and stop bleeding after a tooth extraction. A horseshoe or U-shaped qube may be configured in a variety of sizes and proportions. FIGS. 4A-4C, 5A-5C and 6A-6C illustrate three example sizes, including a small size for the anterior teeth region, a medium size for the premolar teeth region, and a large size for the molar teeth region.

An unique horseshoe shape advantageously allows for precise placement and firm retention over an extraction socket. A compression qube may be configured in size and shape to hug advantageously the topography of the alveolar ridge of both the maxilla and mandible when it is placed. The pore size and reticulated nature of the foam material in an example embodiment also allows for the material to further compress and pressurize over the extraction site when the patient is advised to close/bite down for an initial 20-25 minutes. The additional compression action may reapproximate the soft tissue and bone of the tooth socket (which have been distended following tooth extraction) back to or close to their original positions which reduces post-operative pain, inflammation and ultimately expedites healing. The foam material does not adhere to the surface of wound sites, especially after compressing the wound for initial hemostasis so when a compression qube is removed it does not pull away the initial fibrin clot that forms. This reduces prolonged bleeding. This also helps form and protect a very stable fibrin clot which serves as the foundation for primary wound healing of the extraction socket. In addition, a compression qube in accordance with example embodiments may be configured to absorb fluids including blood and saliva and can be easily wrung out and reused.

A C Qube in another example embodiment also does not leave small cotton fibers behind in the wound the way traditional gauze can do which can cause inflammation as the site heals or prolonged healing time. The C Qube can also be placed over surgical grafting sites after the graft/GTR/sutures are placed to decompress the site, concentrate the growth factors and protect/promote the fibrin clot formation that occurs over the site.

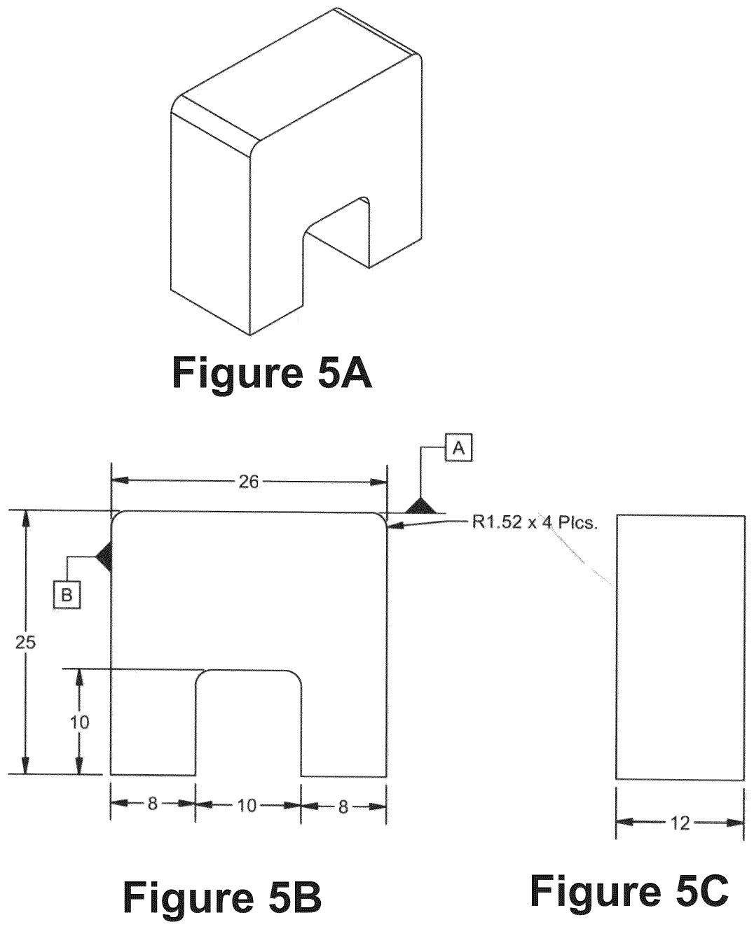

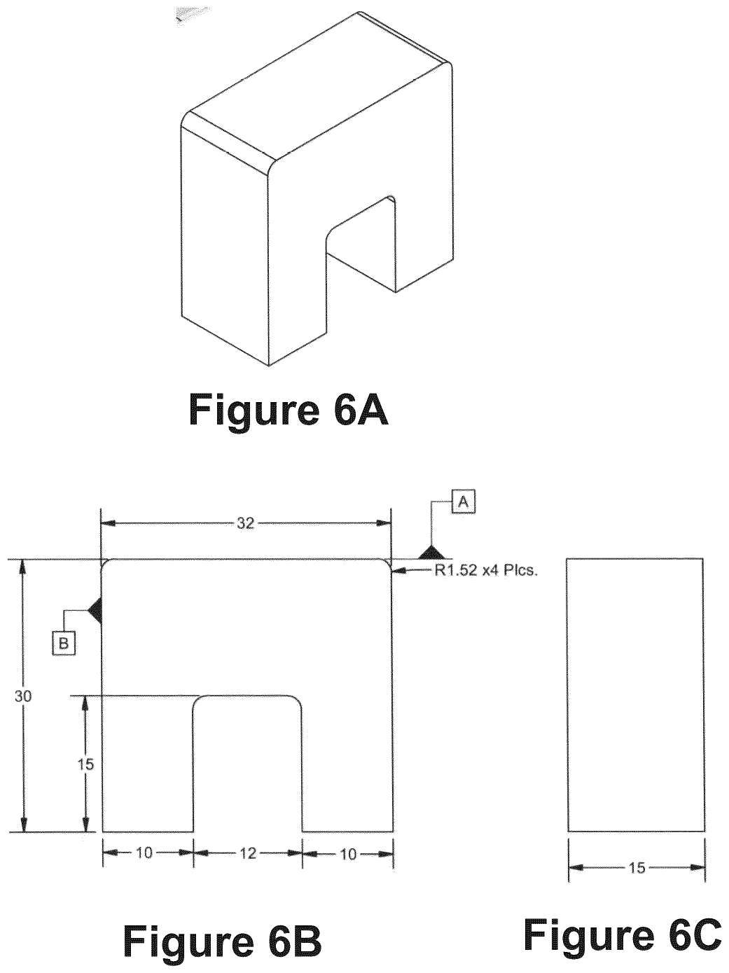

The compression qube or C series qube or C qube may be used advantageously as a cotton gauze replacement and can be used to decompress a wound site and stop bleeding after a tooth extraction or other surgery. This is a very common use of cotton gauze now. The horseshoe or U-shaped C Qube illustrated schematically in the examples illustrated schematically in FIGS. 4A-4C, 5A-5C and 6A-6C, respectively, may be provided individually and/or in kits that include two or more sizes, such as three sizes: Small as in FIGS. 4A-4C for the anterior teeth region, Medium as in FIGS. 5A-5C for the premolar teeth region, and Large as in FIGS. 6A-6C for the molar teeth region.

FIG. 21A illustrates another example embodiment of a U-shaped compression qube that may be a polyether polyurethane foam material including a pore density of 50-100 ppi.

FIG. 22B illustrates another example embodiment of a U-shaped compression qube that in an example embodiment is non-reticulated, felted, and has a pore density of 40-90 ppi.

### G Series Grafting Qubes

A G Series or grafting Qube may be used as an aid for the placement of bone graft material into a bony defect in the oral cavity. Currently, cotton gauze or cotton pellets are what is mainly used as an aid. The unique shape and texture of the G series or grafting qube material helps with placement of bone graft material (human allograft, xenograft, autograft or alloplastic/synthetic graft material) which can be completed immediately following tooth extraction (socket preservation) or during grafting procedures that are completed at a later time such as ridge augmentation, lateral and vertical sinus lifts, and block grafting to name a few. The graft site is usually bleeding (as this is required for successful outcome as the bleeding graft site provides the majority of growth factors and cellular components required for proper wound healing and regeneration of new bone formation). There is often background saliva in the field attempting to enter/wash into the site as well which essentially acts as a contaminant. Once the bolus of bone graft material is delivered with a surgical spoon or other tool, the Grafting Qube may be applied over the bolus of graft material to draw out excess liquid. The excess liquid can also be drawn out through the G Qube when a suction tip instrument is applied. This draws out the excessive liquid while protecting/holding the bone graft material/growth factors in place, within the socket. This prevents the graft material from washing out of the socket, concentrates the growth factors and clotting agents in the wound and prevents saliva from contaminating the wound/graft site. Traditional gauze when used for this application leaves microfibers of cotton behind in the graft site and can get stuck into the suction tip. The graft material can also stick to the gauze and be pulled away from the site when the gauze is removed.

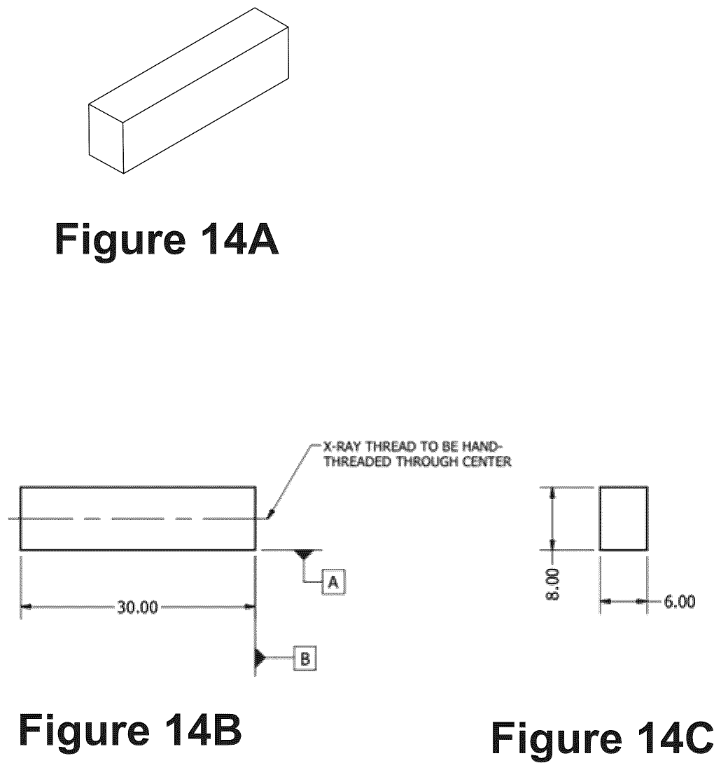

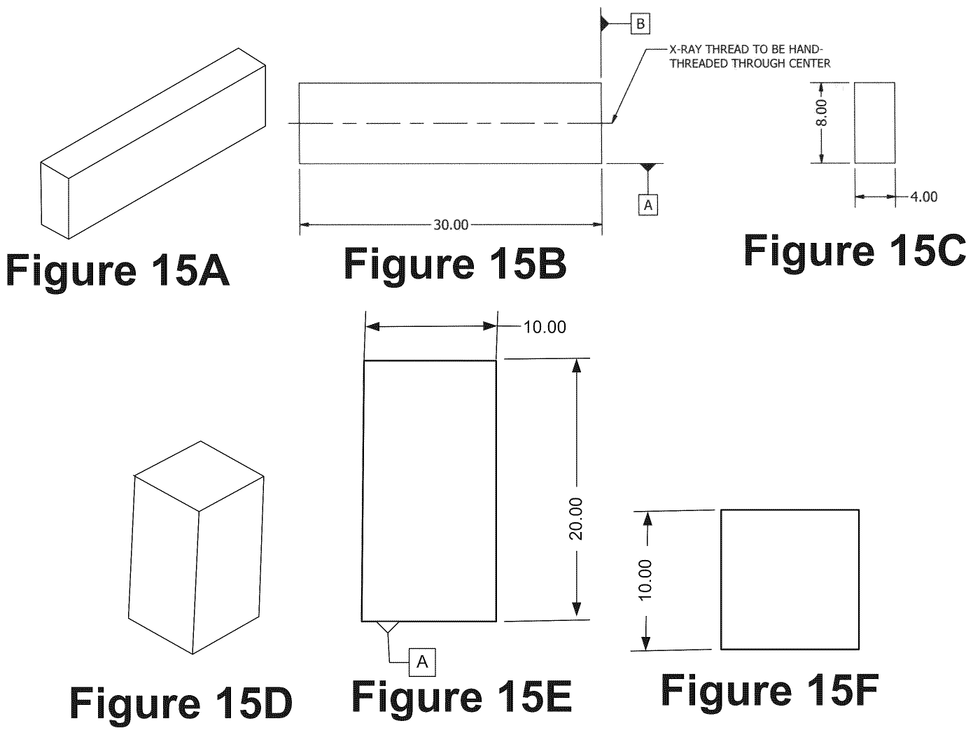

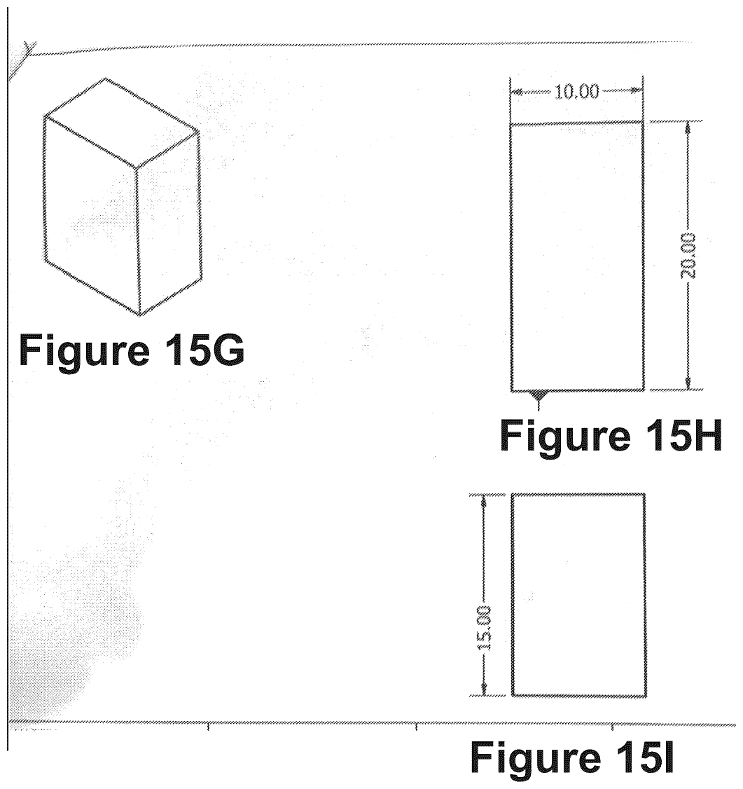

A Grafting Qube or G series qube may be used as an aid for the placement of bone graft material into a socket in an oral cavity where a tooth has been extracted and where new bone is desired to be set in the oral cavity in order to do a future tooth replacement. Currently, cotton gauze is what is mainly used as an aid in this regard. Different shapes and sizes may be used. FIGS. 14A-14C and 15A-15I schematically illustrate examples of G series foam rectangular qubes of relatively small, medium and large sizes and/or fat to thin sizes and/or tall to short sizes.

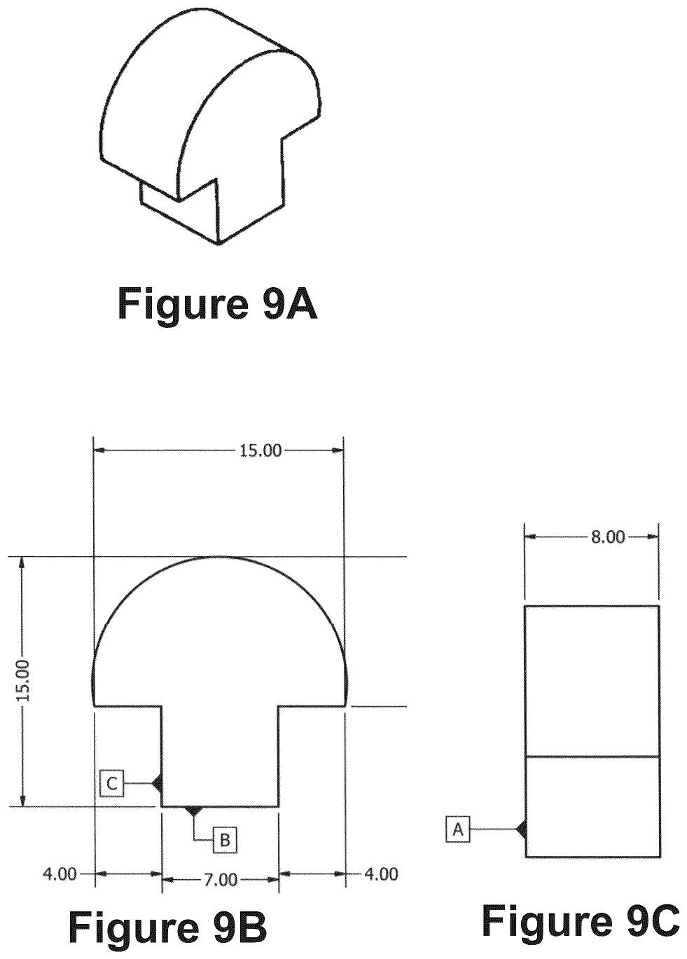

FIGS. 9A-13C and 16A-20 schematically illustrate grafting qubes, or qubes that may be used during certain steps of a grafting procedure, in accordance with example embodiments. Polyester or polyether polyurethane foam, or another material recited above or below herein, or variations thereof in their starting chemistry and/or final packaged configuration, may be selected as a starting material in example embodiments. The starting material may be formed as one or more sheets having one or more particular thicknesses and one or more pore densities. Certain two-dimensional shapes may be cut-out or stamped out from the starting sheets. These unfelted or uncompressed qubes may then be felted or compressed to higher density within a range of higher densities from unfelted or uncompressed to up to five, six or seven times the unfelted or uncompressed density of pores and/or molecular materials. The felted or unfelted qubes may also be scored through one surface or through two opposite surfaces, or through each of two adjacent surfaces (e.g., perpendicular surfaces of a rectangle) with or without also scoring either opposing surface (e.g., parallel surfaces of a rectangle).

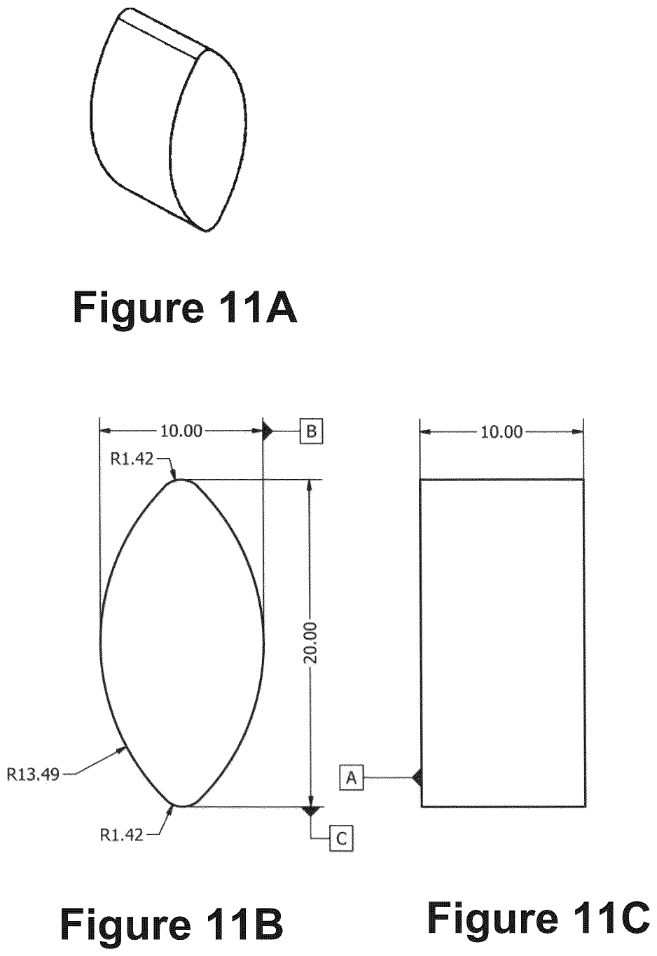

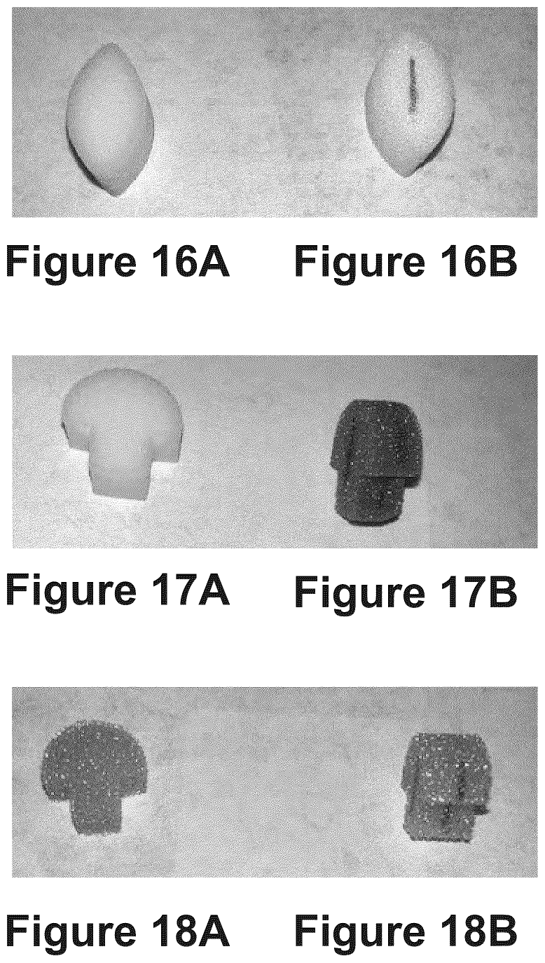

FIGS. 11A-11C and 16A-16B schematically illustrates a quasi-football shaped grafting qube in accordance with an example embodiment. By quasi-football shaped, it is meant that in at least one dimension of the three spatial dimensions, the quasi-football shaped qube is not truly football shaped. In one example embodiment, a football-shaped qube is curved symmetrically in two dimensions around its long axis with a radius of curvature that is larger than that of a spherical ball such as a soccer ball or a basketball. Looking along the direction of the long axis of a football-shaped qube, any cross section of the football-shaped qube is circular, wherein the circles have minimum or even zero radii at the long axis ends of the football and a maximum radius in the center.

The example qube embodiments of FIGS. 11A-11C and 16A-16B are referred to as exhibiting a quasi-football-shape, because the radius of curvature is not the same in all directions 360° around the long axis of the quasi-football. The quasi-football shaped qube of FIG. 16A is curved like a football in only one of two dimensions that are orthogonal to the long axis of the quasi-football. In the second dimension, there may be no curvature, as in the example of FIG. 16A. In this no curvature example embodiment, looking along the direction of the long axis of the quasi-football-shaped qube example of FIG. 16A, cross-sections are rectangular, wherein the rectangles have maximum elongation at the long axis ends of the football and a minimum elongation or even zero elongation, i.e., a square cross-section, in the center.

In the quasi-football shaped qube example embodiment of FIG. 16A, the curvature in the first dimension may be constant and symmetrical about a plane that includes the long axis and the non-curved perpendicular axis, e.g., like a cylindrical lens viewed from a side in a direction orthogonal to the optical axis. Continuing with the optical lens analogy, quasi-football shaped qubes may have opposing same or different spherical or aspherical curvatures on either side of a center plane that includes the long axis of the quasi football.

In another alternative example embodiment, a quasi-football shaped qube may have a more or less pear shape which is not symmetrically wide above and below the “50 yard line” or “equator” or center plane that is equidistant from each end of the quasi-football-shaped qube along its long axis. The curvature in example embodiments may even be opposite in segments to a true football such as with the curvature of an hourglass, a saddle or a barbell.

FIG. 16B schematically illustrates a quasi-football shaped grafting qube that is scored in accordance with an example embodiment. The quasi-football shaped grafting qube of FIG. 16B exhibits a football shape in a first plane that includes the long axis of the quasi football, while in a second plane that also includes the long axis of the quasi-football and is orthogonal to the first plane, the quasi football shaped qube includes a thin elongated cut that may be between 0.5 mm and 1.5 mm deep, or between 0.5 mm and 5.0 mm deep, such as 1.0 mm, 1.5 mm, 2.0 mm, 2.5 mm, 3.0 mm, 3.5 mm, 4.0 mm, or 4.5 mm. The cut may be arbitrarily thin and elongated, or may have little or no elongation, or something in between zero width and a width that approaches 1/16, 1/12, 1/10, ⅛, ⅙, ¼, ⅓ or ½ or more of the length of the cut. The length of the cut may be between 3-5 mm and 8-10 mm.

FIGS. 9A-9C, 10A-10C, 17A-17B and 18A-18B schematically illustrate example embodiments of quasi-mushroom shaped grafting qubes. As with the quasi-football shaped examples qube of FIG. 16A-16B, by quasi-mushroom-shaped, it is meant that in at least one dimension of the three spatial dimensions, the quasi-mushroom shaped qube is not truly mushroom shaped.

In one example embodiment, a mushroom-shaped qube at one end is curved symmetrically in two dimensions around its long axis with a radius of curvature that may be the same or nearly the same as a spherical ball such as a soccer ball or a basketball, while the other end includes a cylinder, cube or combination thereof, tapered or untampered, truncated or not truncated, curved or flat or a combination thereof at the edges in the direction of the long axis and/or in any direction of a plane perpendicular to the long axis. Looking along the direction of the long axis of a mushroom-shaped qube, any cross section of the mushroom-shaped qube at the first end is circular, wherein the circles have a minimum or even zero radius at the long axis end of the first end of the mushroom and a maximum radius at the base of the first end of the mushroom. The second end of the mushroom may have a smaller and more constant cross section end to base than the first end.

The example qube embodiment of FIG. 17A is referred to as exhibiting a quasi-mushroom-shape, because the radius of curvature is not the same in all directions 360° around the long axis of the quasi-mushroom at least at the first end. The quasi-mushroom shaped qube of FIG. 17A is curved at the first end like a mushroom in only one of two dimensions that are orthogonal to the long axis of the quasi-mushroom. In the second dimension, there may be no curvature, as in the example of FIG. 17A, or there may be an asymmetric curvature. In the no curvature example embodiment, looking along the direction of the long axis of the quasi-mushroom-shaped qube in the example of FIG. 17A, the cross sections are rectangular, wherein the rectangles have maximum elongation at the long axis end of the mushroom and a minimum elongation or even zero elongation, i.e., a square cross-section, at the base, i.e., wherein the two ends of the mushroom meet. The second end of the quasi-mushroom shaped qube of FIG. 17A may have a more or less true mushroom shape with circular cross sections or may exhibit one or more sharp vertices and/or flat edges as may be efficiently and conveniently machined.

In the quasi-mushroom shaped qube example embodiment of FIG. 17A, the curvature at the first end in the first dimension may be constant and symmetrical about a plane that includes the long axis and the non-curved perpendicular axis, e.g., like a cylindrical lens surface viewed from a side in a direction orthogonal to the optical axis. Continuing with the optical lens analogy, quasi-mushroom shaped qubes may have opposing same or different spherical or aspherical curvatures on either side of a center plane that includes the long axis of the first end of the quasi mushroom.

In another alternative example embodiment, a quasi-mushroom shaped qube may have a more or less pear shape which is not uniformly curved base to end along its center axis. The curvature in example embodiments may even be opposite in segments to a true mushroom such as with the curvature of an hourglass, a saddle or a barbell.

FIG. 17B schematically illustrates a large quasi-mushroom-shaped grafting qube that is scored in accordance with an example embodiment. The quasi-mushroom of FIG. 17B exhibits a mushroom shape in a first plane that includes the long axis of the quasi mushroom, while in a second plane that includes the long axis of the quasi-mushroom and is orthogonal to the first plane, the quasi mushroom shaped qube is cut in this example embodiment. The cut may be arbitrarily thin and elongated, or may have little or no elongation, or something in between zero width and a width that approaches 1/16, 1/12, 1/10, ⅛, ⅙, ¼, ⅓ or ½ or more of the length of the cut. A cut may be provided within any of the four sides of the quasi-mushroom shaped qube of FIG. 17B.

FIGS. 18A-18B schematically illustrate small quasi-mushroom shaped grafting qubes without scoring and with scoring, respectively, in accordance with example embodiments. The discussion with regard to large mushroom and large quasi-mushroom shaped qubes with reference to FIGS. 17A-17B is incorporated here by reference.

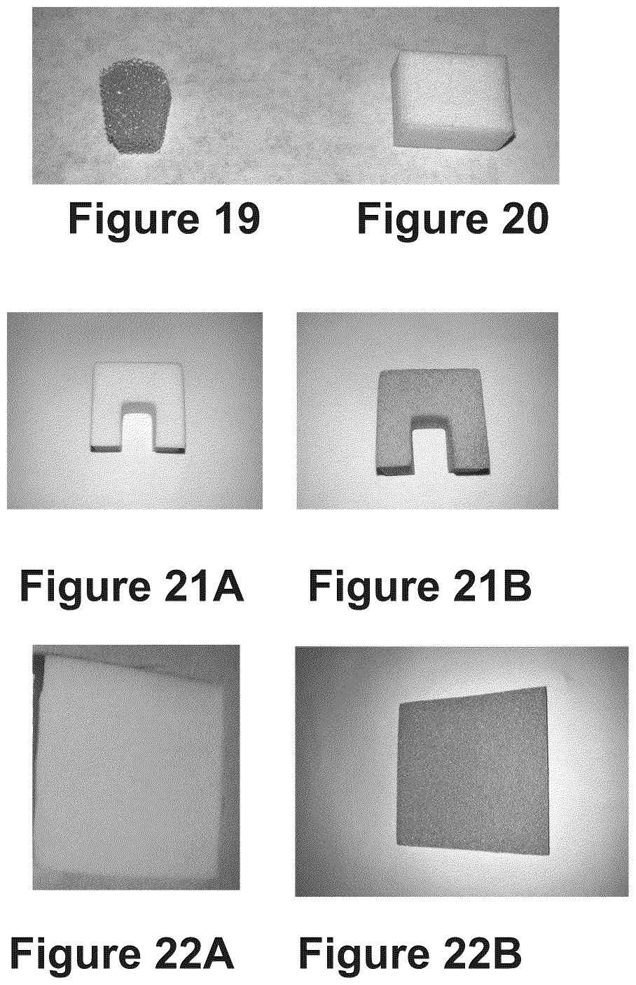

FIG. 19 schematically illustrates a quasi-truncated cone-shaped grafting qube that is scored in accordance with an example embodiment. Examples of differences between a true truncated conic section and a quasi-truncated cone are incorporated here by reference to the above discussions.

FIG. 20 schematically illustrates a felted rectangular dab qube in accordance with an example embodiment. The dab qube of FIG. 20 may be triple felted or may be three times more dense compared with an unfelted qube, or in a range of 2× to 5× in example embodiments. Moreover, an increase in the density of an unfelted qube has been provided by felting or compressing a 1× density qube that has been formed in a separate manufacturing process.

Placement of bone graft material (e.g., human allograft, xenograft, autograft or alloplast synthetic graft material) can be completed immediately following tooth extraction (socket preservation) or grafting procedures that are completed at a later time such as ridge augmentation, lateral and vertical sinus lifts, and block grafting to name a few. Conventional graft materials have a consistency of ground up corral particles or large particles of sand, which is often mixed with a liquid to serve as a carrier such as sterile saline or blood particulate from the patient, PRP mixtures, PRF mixtures, Emdogain or other growth factors. During graft placement, the graft site may also be bleeding as this is actually desired for successful outcome as the bleeding graft site may be relied upon to provide a majority of growth factors and cellular components for proper wound healing and regeneration of new bone formation. There can also be background saliva in the field attempting to enter the site as well which is essentially a contaminant. Once the graft site receives a bolus of bone graft material that is delivered with a surgical spoon or other tool, the Qube can be advantageously used to help hold the powdered bone material in place, rather than quickly washing out of a tooth socket from blood and saliva. The doctor may be trying to create new bone in an area where a tooth was pulled and will eventually be replaced by an implant screw and crown that is screwed into the jaw.

The shape, size and proportions of a Grafting Qube or G series qube may be advantageously selected for its use as a tool and ergonomic handling ability.

In one example process, a 1× density polyether polyurethane or polyester polyurethane qube, or another material recited above or below herein, may be formed in a process involving combining one or more isocyanates, diisocyanates, or aromatic, aliphatic or cycloaliphatic isocyanates, with one or more polyols, such as, e.g., a polyether polyol or a polyester polyol, to form a polyether polyurethane or a polyester polyurethane starting bulk volume. Sheets of selected thickness may be cut and rolled from the starting bulk volume. One or more of a variety of qubes of different shapes, sizes, porosities, or molecular weights, or combinations of these or other material characteristics, traits and/or properties may be manufactured. In an example embodiment, a starting polyether or polyester polyurethane bulk volume may be generated that has a molecular weight of 2000 or more. Those 1× density qubes may then be up to 2×, 3×, 4×, or −5× felted or compressed.

In further example embodiments, polyether or polyester starting foam materials may be varied in molecular and/or pore density in one, two or all three spatial dimensions along regular or angularly offset polygonal (e.g., rectangular, pyramidal, trapezoidal, conic) axes. Besides triple felting, in other example embodiments, dab qubes and/or grafting qubes may be unfelted, double felted, quadruple felted or quintuple felted, and/or 1× compressed (i.e., not compressed), 2× compressed, 3× compressed, 4× compressed, 5× compressed, or up to 5×-6× felted or compressed.

In example embodiments, grafting qubes may be formed from polyether foam having a pore density in a range between 45 pores per inch (ppi) and 110 ppi, or between 55 ppi and 100 ppi, or between 65 ppi and 90 ppi, or between 70 ppi and 85 ppi, or between 72 ppi and 82 ppi, or between 73 ppi and 81 ppi, or between 74 ppi and 80 ppi, or between 75 ppi and 79 ppi, or between 76 ppi and 78 ppi or in a range around 77 ppi.

In other example embodiments, dab qubes may be formed from polyester foam material, e.g., polyester foam that may be felted or compressed to double, triple, quadruple or quintuple its pore density. Examples of dab qubes or dab sponges may include cubes or may have other rectangular, triangular, pyramidal, trapezoidal, oval, mushroom, football, thimble, conic or truncated conic, meniscus, lenticular, cylindrical, rhombohedral, tertrahedral, quadrahedral, pentahedral, octahedral, or other curved or polygonal shapes or combinations thereof, which may be reticulated or non-reticulated, and may have a pore density in a range between 45 pores per inch (ppi) and 155 ppi, or between 55 ppi and 145 ppi, or between 65 ppi and 135 ppi, or between 75 ppi and 125 ppi, or between 85 ppi and 115 ppi, or between 90 ppi and 110 ppi, or between 95 ppi and 105 ppi, or between 97 ppi and 103 ppi, or between 98 ppi and 102 ppi or between 99 ppi and 101 ppi, or in a range around 100 ppi.

Thin square qubes, such as those illustrated schematically in FIGS. 2A-2C, 3A-3C and/or 22A-22B may be particularly useful as a gauze replacement or gauze substitute. These square qubes may be four inches on each side, or three inches on each side, or two inches on each side, or one inch on each side, or 1-20 cm on each side, e.g., 5 cm, 10 cm or 15 cm, or the gauze replacement qubes may be rectangular, e.g., with a length to width ratio of 1, 1.5, 2, 2.5, 3, 4, 5 or up to 10 or more, or circular or elliptical, or another polygonal or curved shape or a combination of regular and/or irregular shapes. Such gauze replacement qubes may be sterilized and packaged individually or with a coupling mechanism for holding in place at a wound site, grafting site, retraction site, compression site, an osseointegration site, a blistering or callusing site, a layering, cushioning and/or thermalizing site, or another site wherein cotton gauze may have been historically useful for softening, absorbing, retracting, spacing, or otherwise. The thicknesses of qube squares may be 0.8 mm, 0.9 mm, 1 mm, 1.25 mm, 1.5 mm, 1.75 mm, 2 mm, 2.5 mm, 3 mm, 4 mm, 5 mm, 6 mm, 8 mm, 10 mm or otherwise depending on the application, e.g., thicker as an insulator and thinner as a filter or suction biasing agent.

### MS Series Qubes

Qube MS/Dab, in another example embodiment, may be configured to replace cotton gauze or cotton pellets and perform advantageously to cleanse a tooth extraction site or a wound site in an oral cavity. An example embodiment of an MS/Dab Qube may be configured advantageously to allow for absorption of a liquid such as chlorohexidine or povidone iodine which can be carried to a contaminated extraction site and be inserted into the site. The shape and material properties of an MS Qube may be selected and the MS Qube may be advantageously configured to allow such MS Qube to conform to an internal socket shape while holding a disinfecting solution. An MS Qube in another example embodiment can be placed and then easily retrieved without tearing or disintegrating. An MS Qube in accordance with another example embodiment can also be used to scrub socket walls or other surgical sites. The ergonomic shape of an MS Qube can be configured in an example embodiment to allow for improved handling and reduced working time making the practitioner more efficient and precise.

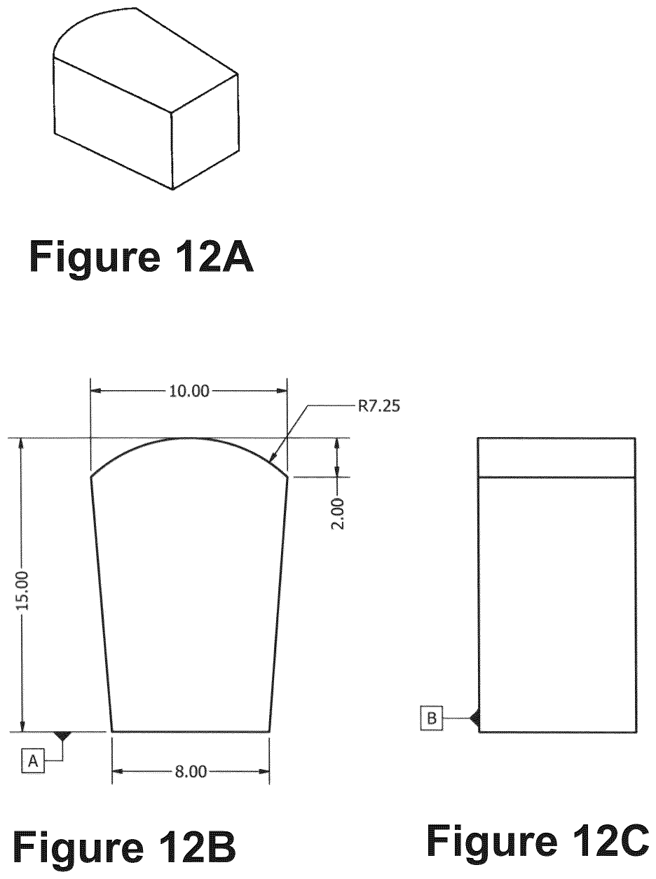

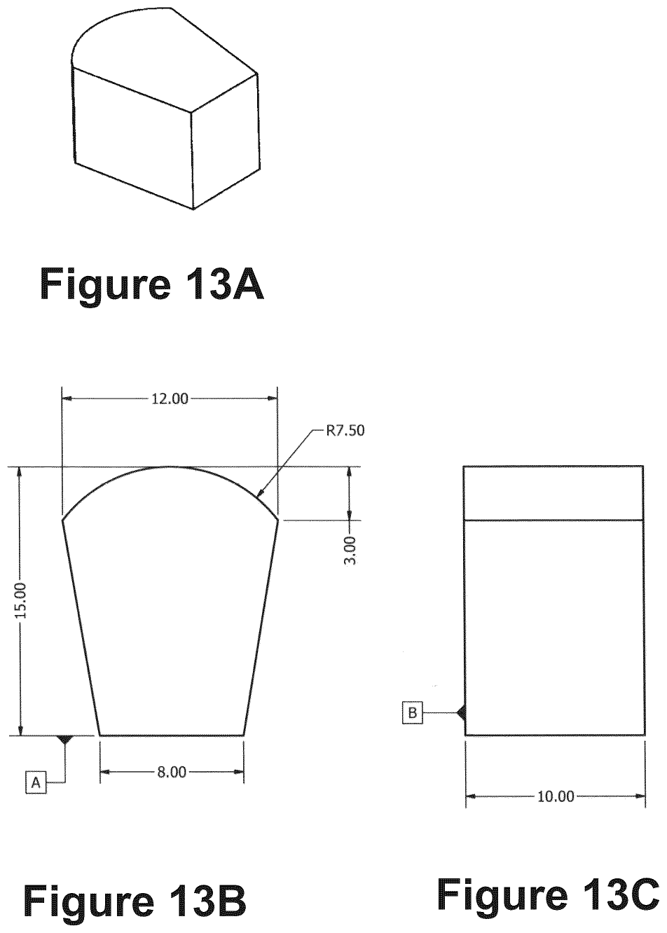

An MS series qube may have a 2D thimble shape and may serve advantageously as a cotton gauze replacement providing a convenient shape for surgical or non-surgical medical use when cleaning a skin area or other bodily area or applying a sterile or sterilizing solution, a medicament or absorbing or moving excess fluids. This 2D thimble shape may be an option for use in a grafting procedure, as a G series qube. The thimble shape may be 2D or 3D and may be configured in a variety of sizes, such as the relatively smaller and larger sized examples of FIGS. 12A-12C and 13A-13C, respectively. The radii of curvature of the top surfaces in the examples of FIGS. 12A-12C and 13A-13C are 7.25 mm and 7.5 mm, respectively, although these radii may be more or less than in these examples, such as 4 mm, 5 mm, 6 mm, 7 mm, 7.75 mm, 8 mm, 9 mm or 10 mm or more. The smaller radii of curvature would accompany smaller widths and the larger radii would accompany larger widths, at least at the top, whereas more or less tapering top to bottom than 20% or 33%, as in the illustrated examples, may be provided in further example embodiments. The base of an MS series qube may be square as in the example embodiment of FIGS. 12A-12C or rectangular as in the example embodiment of FIGS. 13A-13C. In other example embodiments, the base may be trapezoidal, circular, elliptical, triangular, pentagonal, hexagonal or another polygonal shape and/or a shape having one or more sides that are partially-curved or wholly-curved, convex or concave, or other regular or irregular shapes.

In some example embodiments, a medicated qube or MS qube may be reticulated, and double or triple felted, and have a relatively high starting pore density between about 60-70 ppi and 130-140 ppi or in one example about 100 ppi prior to the double or triple felting. In some examples, a MS qube may be rectangular on at least four of six sides, like a shipping container.

In other example embodiments, a medicated qube or MS qube may be non-reticulated, unfelted and have a starting pore density between 40 ppi and 110 ppi, or in one example around 77 ppi. In some examples, a MS qube may include a rectangular polyhedron which may be rectangular on the four sides that are parallel, in one of its two dimensions, with the long axis of the rectangular polyhedron.

In an example embodiment, a non-reticulated, unfelted medicated qube or MS qube may have a starting pore density between 30-50 ppi and 110-130 ppi or in one example about 77 ppi. This example MS qube may have dimensions 1 cm×1 cm×3-5 mm. In another example embodiment, a medicated qube or MS qube may be reticulated and may have a starting pore density between 30-50 ppi and 110-130 ppi or in one embodiment around 77 ppi.

### R Series Retraction Qubes

R series Qube, in another example embodiment, may be configured to replace cotton gauze pads and/or stainless steel retraction instruments and perform advantageously to retract soft tissue during oral surgical procedures in the oral cavity. During implant surgery, a surgical incision is created along a surface of a ridge in a maxilla or mandible in the site of a prosed implant. A gingival tissue flap may be then retracted back away from the ridge to expose a bony ridge beneath. The gingival tissue flap may be then retracted with an R series Qube throughout the procedure. Conventional surgical stainless steel instruments can create trauma to tissue during a procedure. Traditional gauze, e.g., cotton gauze, can also be used but has limited ability to retract and often leaves fibers in the surgical site. Such cotton gauze also tends to pull clotting factors away from the site increasing bleeding. The R Qube retracts the tissue in a way that allows for firm yet gentle contact with oral tissues while not tending to leave fibers at the surgical site nor tending to pull clotting factors from the site when removed.

Example embodiments of relatively larger and smaller sized cotton gauze replacement qubes are, illustrated schematically at FIGS. 14A-14C and 15A-15I. The cotton gauze replacement qubes can be used instead of mechanical means or the insertion of cotton gauze on a temporary basis, e.g., to help keep a jaw bone and a patient's gum material separated during a surgical procedure. In an example embodiment, a thin rectangle shaped R series qube may be unfelted, although in further example embodiments, thin rectangle or rounded elongated qubes may be felted or unfelted.

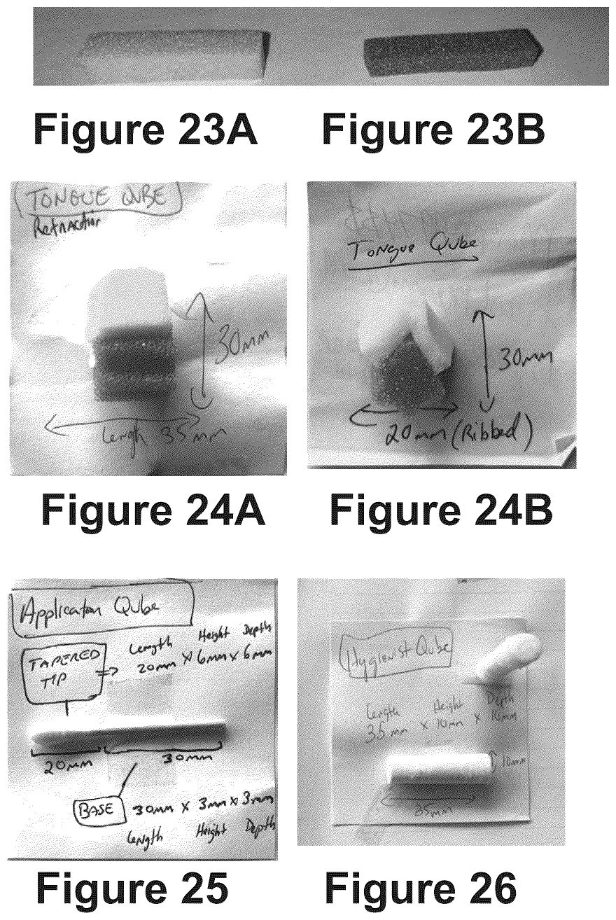

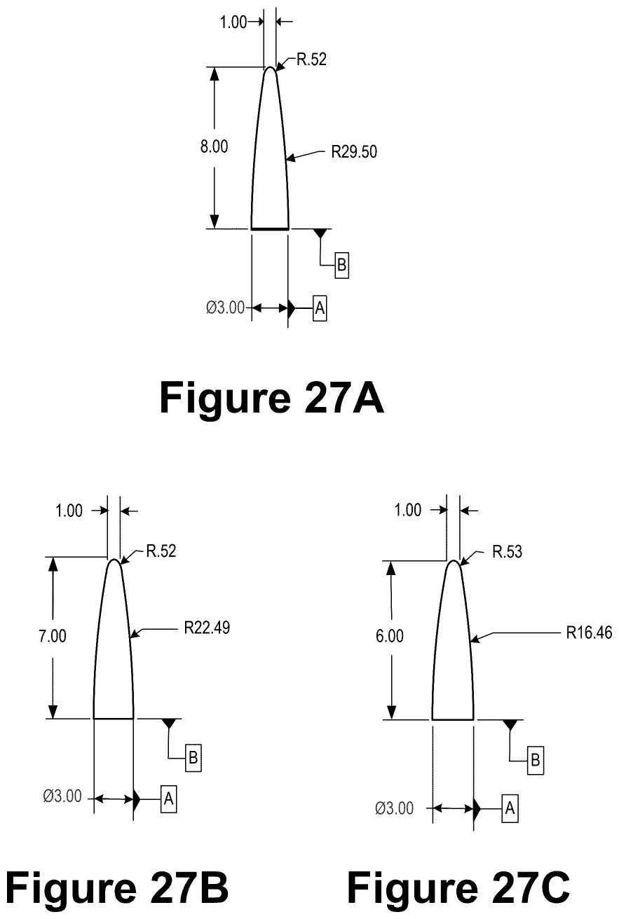

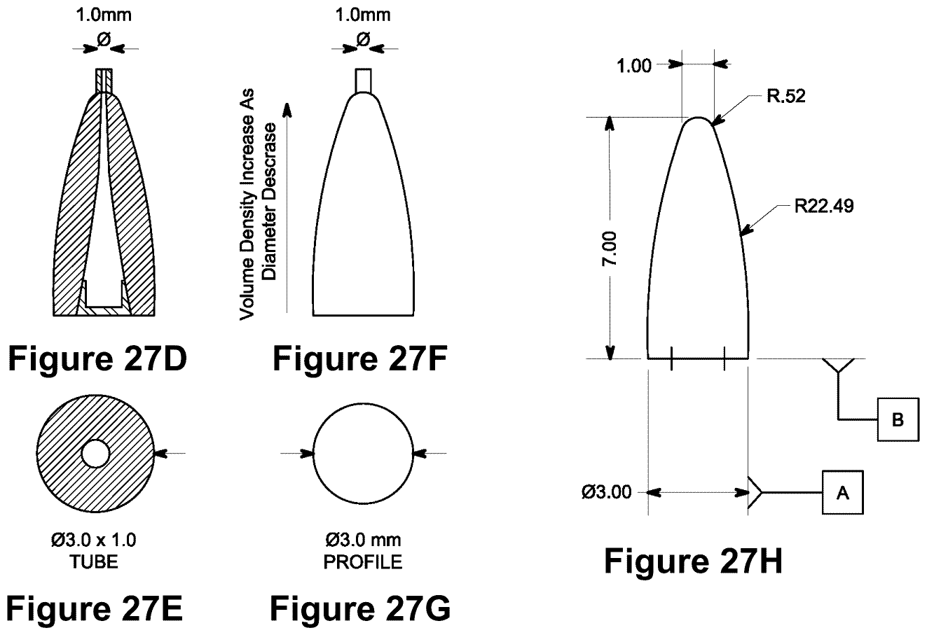

These thin rectangle or otherwise elongated R series shapes (see also, e.g., FIGS. 23A and 23B) may be configured in a variety of shapes, sizes and proportions. For example, R series Qubes in accordance with example embodiments may have symmetric cross sections such as squares, circles or squares with rounded corners, or asymmetric cross sections such as ellipses, rectangles, rectangles with rounded corners, other polygons, or shapes including one or more convex or concave sides with or without rounded corners. In further example embodiments, the length may be selected to match a depth of a patient's jaw bone which may be more or less than the 30 mm shown in the examples of FIGS. 14A-14C and 15A-15I. Thicknesses smaller than 4 mm, such as 1 mm, 1.5 mm, 2 mm, 2.5 mm, 3 mm, or 3.5 mm may be provided in example embodiments, and thicknesses larger than 6 mm may be provided, such as 7 mm, 8 mm, 9 mm or 10 mm or more, and thicknesses between 4 mm and 6 mm may be provided such as 4.5 mm, 5 mm or 5.5 mm. The height may be more or less than the 8 mm shown in FIGS. 14A-14C and 15A-15I, in further example embodiments, as well, such as between 3-4 mm and 15 mm or more. The R series foam rectangular qube examples illustrated schematically in FIGS. 14A-14C and 15A-15I may include an x-ray thread that may be hand-threaded, or alternatively machine threaded, through the long dimension of the qube or such x-ray thread may be adhered to a surface of the qube or such x-ray thread may be formed through the qube during a curing process or poured and cured or a cavity formed through an R series qube may be provided to fill after curing with an x-ray thread or other x-ray process-specific material.

FIGS. 23A and 23B illustrate soft tissue retraction Qubes, or R-Qubes, in accordance with example embodiments. R series qubes may be reticulated or non-reticulated, and felted or unfelted, and may have had starting pore densities of about 50 ppi-105 ppi, and may be elongated regular or irregular flat or curved shapes. Other example embodiments of R series qubes include retraction qubes that may be non-reticulated, and unfelted, and may have had starting pore densities of about 60 ppi, and may be elongated regular or irregular flat or curved shapes.

### More Qubes Configured for Different Functions

FIGS. 24A-26 illustrate example embodiments of Qubes performing advantageous additional functions and/or in different site locations. In the example embodiments illustrated at FIGS. 24A-26, each Qube may be made from polyester materials or from polyether materials, in felted or unfelted forms, and in reticulated or non-reticulated forms. Pore sizes may vary from 45-120 ppi. This provides a wide latitude of uses and applications.

### Tongue Retraction Qube

FIGS. 24A-24B illustrate an example embodiment of a Tongue Retraction Qube, or tongue retractor qube, in side and front views, respectively. FIGS. 24A-24B illustrate a tongue retraction qube that may be non-reticulated, and unfelted, and may have had starting pore densities of 40-80 ppi, or 50-70 ppi, or 55-65 ppi, or in a narrower range around about 60 ppi, and may be elongated regular or irregular flat or curved shapes.

A tongue retraction Qube, such as in the example of FIGS. 24A-24B, may be configured with a shape and texture that is made of nonstick, porous foam that has good tensile strength, good capacity for absorption of saliva, and firm, gentle stability against soft tissues. The shape of a tongue retraction Qube in example embodiments advantageously allows it to be placed along the floor of a mouth beneath the tongue and along the lingual vestibule providing retraction if the tongue, a protective barrier for the rogue during operative procedures, and to absorb saliva and blood during procedures for improved visibility and access to a surgical or op elevating site.

Stationary placement can also provide a constant stabilization of the tongue instead of a manually placed tongue retraction instrument. which may tend to involve varying pressure which can exacerbate a tongue gag reflex increasing gagging during a procedure. The tongue retraction Qube in an example embodiment may be configured to provide a constant form of retraction eliminating the downward force that a manual retraction instrument creates which in turn can tend to induce the gag reflex.

A tongue retraction Qube in accordance with example embodiments may be configured to be atraumatic to soft tissue preventing soft tissue injury and also preventing subsequent traumatic soft tissue post operative ulcerations which can occur from manual instrument retractors. There is less soft tissue injury and post op pain as an expected result of using a tongue retraction qube instead of a conventional manual instrument retractor.

### Applicator Qube

An applicator Qube in accordance with an example embodiment is illustrated at FIG. 25. FIG. 25 illustrates an Applicator Qube that may be double or triple felted, or felted up to five times 5× or six time 6× compression. An Applicator Qube in an example embodiment may be designed such that it can be modified in tip size for small, medium and large sizes with the tip diameter ranging from 2.0 mm to 20.0 mm based on the applications required. The foam material of an example Applicator Qube may be non-stick, non-traumatic to soft tissue, or configured for atraumatic oral surgeries. An applicator qube may be firm in density for applying or removing, and/or may be configured to be porous for absorption and/or to carry one or a combination of medicaments, may have good tensile strength, and/or may be configured to not have a tendency to leave behind fibers or residue like conventional cotton applicators.

The applicator Qube can be configured to be used for a host of applications such as topical anesthetic application, placement of bonding agents, and/or delivery of intraoral medications. An applicator Qube can be configured for use in example embodiments as an aid in placement of materials or restorations into teeth, to burnish a surface layer, remove excess, and/or to apply additional pressure and/or to remove pressure indicating paste for denture adjustments, application of medicaments during surgical procedures, and/or removal of impression materials. When used as a universal tool, there are numerous other applications that dentist will utilize it.

### Hygienist or Qube Roll

FIG. 26 illustrates end and side views of a qube roll in accordance with an example embodiment, which may be similar to a cotton roll except a qube roll is not made of cotton, and may be advantageously configured to provide saliva flow control during a dental surgical procedure. A qube roll in example embodiments may be cylindrical, tubular, and/or otherwise elongated rhombohedral, trapezoidal or rectangular or tubular and 3× felted or otherwise felted up to five times 5× or six times 6× compression. In the example of FIG. 26, the qube roll has a cylindrical shape with a length of about 35 mm and a diameter or about 10 mm. Other example embodiments may include qube rolls between 20 mm and 50 mm, or between 25 mm and 45 mm, or between 30 mm and 40 mm in length, and between 5 mm and 20 mm in diameter.

A Hygienist Qube or Qube roll in accordance with example embodiments may be formed of or from a material such as one or more of the materials identified herein, e.g., polyether polyurethane, polyester polyurethane or ePTFE, or PTFE, that is firm, nonsticky, porous foam that has good tensile strength, exhibits a reliable capacity to absorb saliva, and remain stable in position against, for example, a tongue, throughout a more or less lengthy dental procedure. A hygienist Qube or Qube roll in example embodiments is tissue friendly and protects soft tissue.

The shape of a hygienist Qube or Qube roll may be configured in example embodiments to advantageously allow it to be placed in a vestibule between oral mucosa (i.e., a cheek) and an attached gingival tissue to retract the cheek away from teeth during dental hygiene procedures such as scaling, root and/or prophy polishing. A hygienist Qube or Qube roll can also be used in example embodiments during dental procedures to isolate teeth for preparation and placement of restorations, such as crowns. A hygienist Qube or Qube roll can be configured to absorb saliva and/or blood in the vestibule improving overall visibility and maintaining a dry field especially for moisture-sensitive restorative procedures.

A hygienist Qube or Qube roll in example embodiments can advantageously also be held by hygienists or dentists during hygiene procedures, so that cleaning tools or instruments can be surface cleaned during a procedure. This feature of a hygienist Qube or Qube roll in certain example embodiments may be utilized by wiping an instrument into the foam material which will remove the calculus or tartar from the instrument as a result of selecting a porosity, coarseness and/or tensile strength for the specific procedure or application or tool to be cleansed. The hygienist Qube or Qube roll will not easily tear and the instrument can freely password through it. The hygienist Qube or Qube roll will, in example embodiments, not scratch the instrument and yet will function to clean the surface of the instrument once, twice or several times even regularly throughout a dental cleaning procedure in accordance with example embodiments.

FIGS. 15D-15I illustrate example embodiments of qubes which may be formed from polyether polyurethane material and used as retraction qubes. In another example embodiment, the example qubes illustrated schematically at FIGS. 15D-15I may be formed from polyester polyurethane material and used as “Dab” qubes.

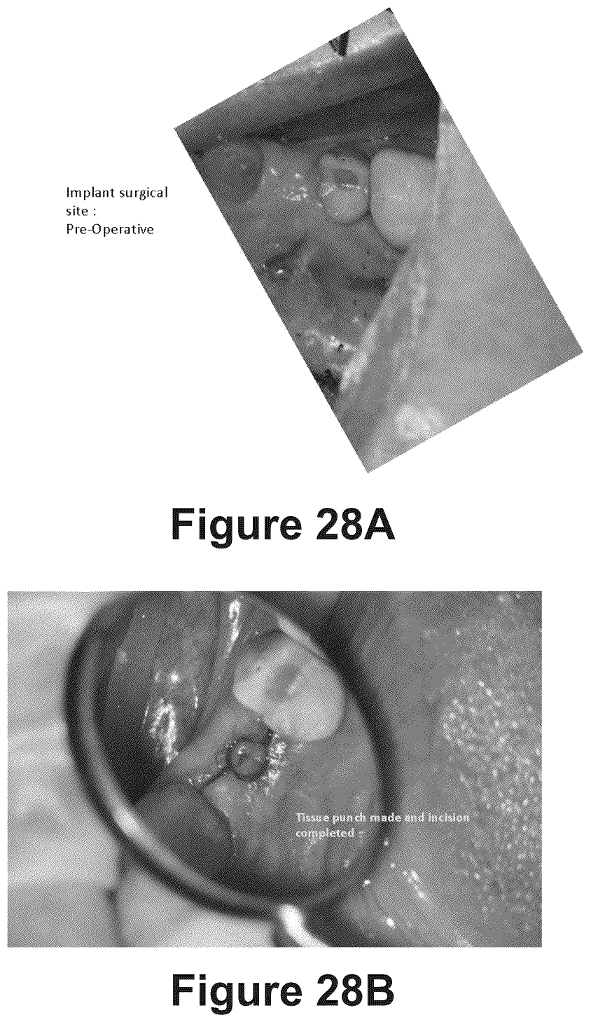

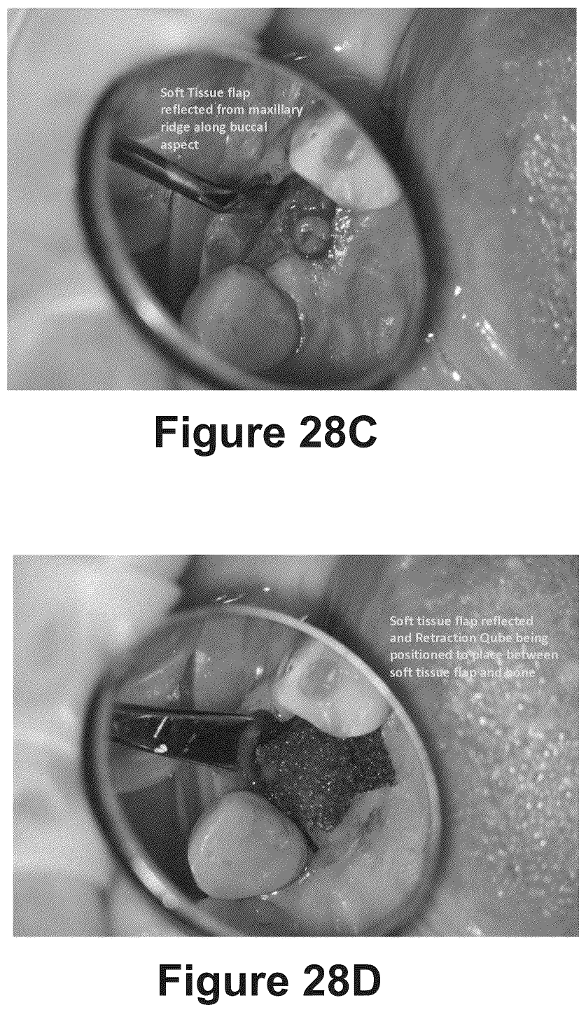

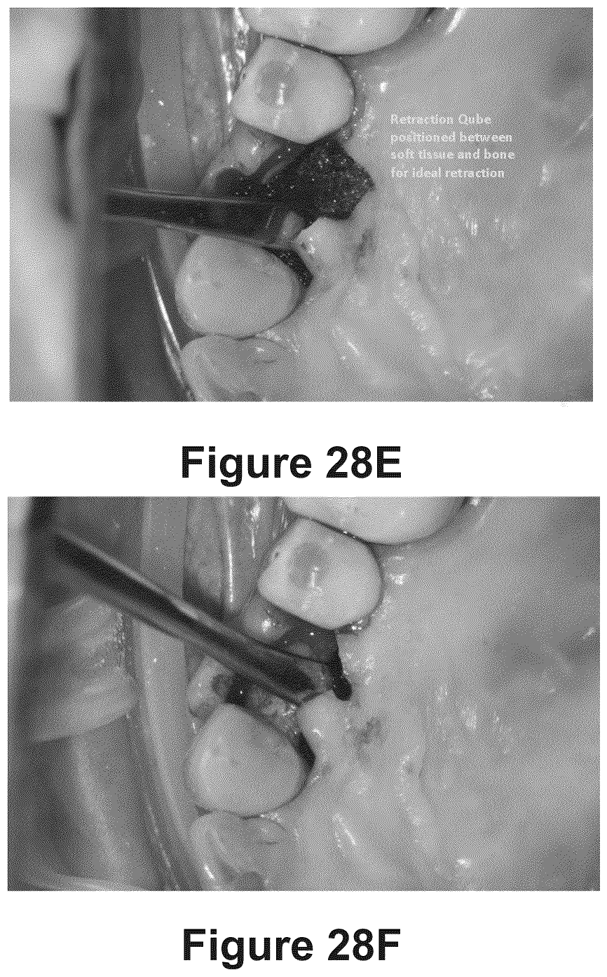

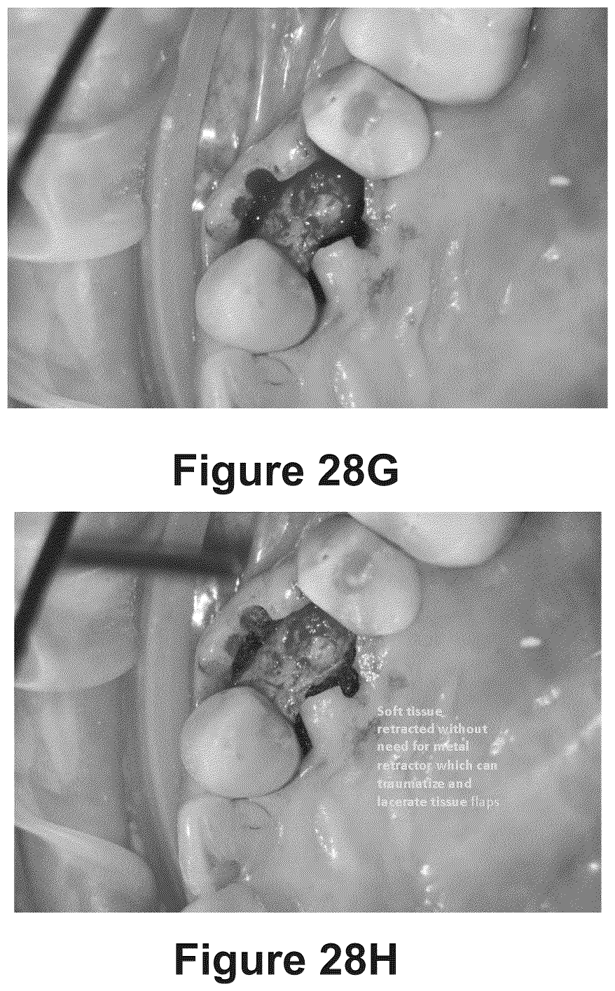

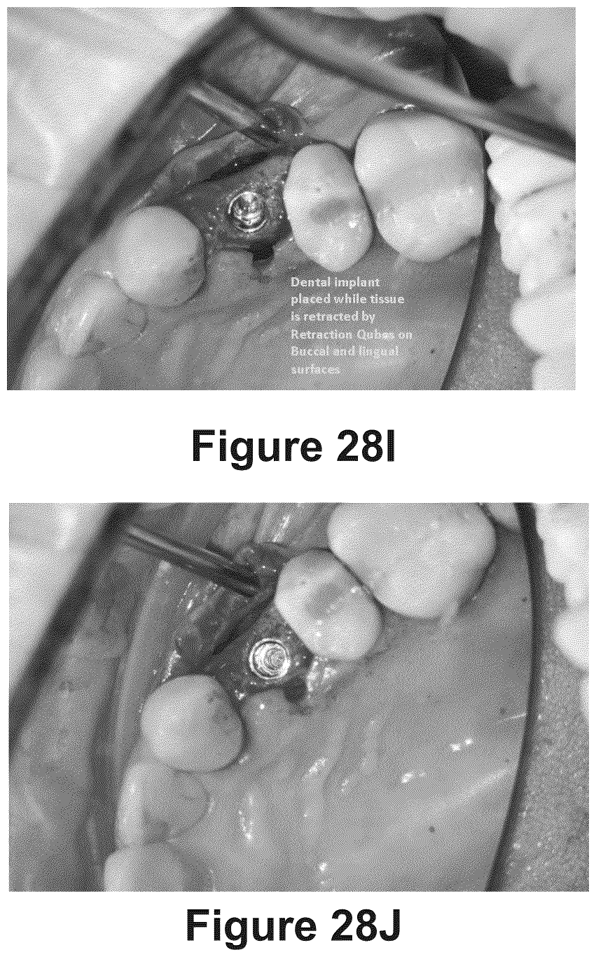

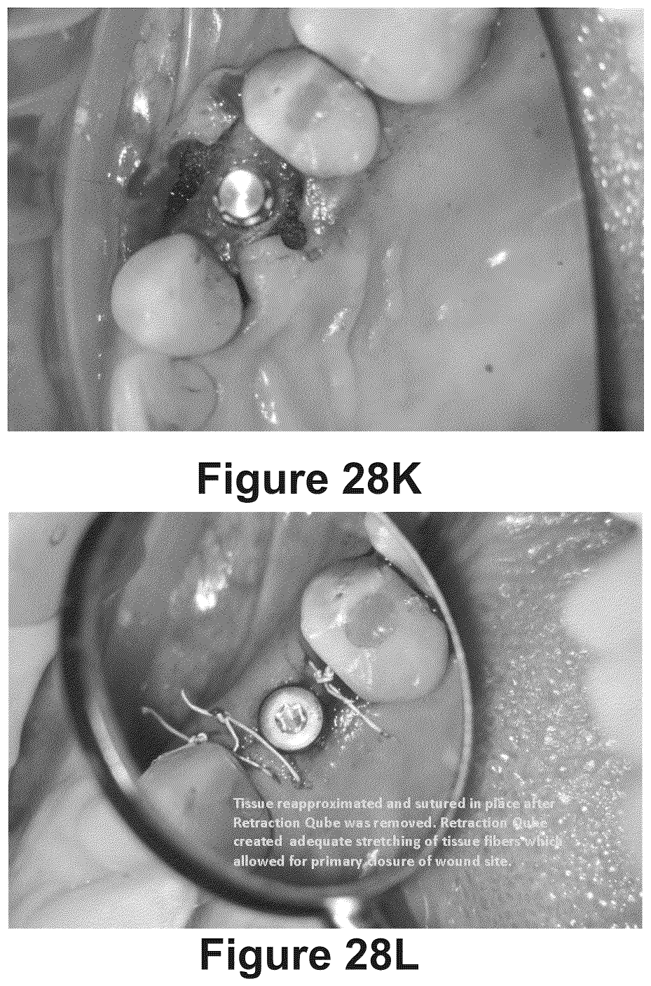

FIGS. 28A-28L illustrate example embodiments of retraction qubes and implant surgery processes.

FIG. 28A is an example of an implant surgical site as it may look before surgery is started.

FIG. 28B shows the same implant surgical site after a tissue punch has been made and an incision completed.

FIG. 28C shows the same implant surgical site with a soft tissue flap reflected from a maxillary ridge along a buccal aspect.

FIG. 28D shows the same implant surgical site with the soft tissue flap reflected and a retraction qube being positioned to place between soft tissue flap and bone.

FIG. 28E shows a retraction qube positioned between soft tissue and bone for ideal retraction.

FIGS. 28F-28G show two further steps in an example implant surgical process.

FIG. 28H shows soft tissue retracted without need for metal retractor which can traumatize and lacerate tissue flaps.

FIG. 28I shows the same implant surgical site with a dental implant being placed while tissue is retracted by retraction qubes on buccal and lingual surfaces.

FIGS. 28J-28K shows two further steps in an example implant surgical process.

FIG. 28L shows the same implant surgical process with tissue reapproximated and sutured in place after a retraction qube was removed, which retraction qube created adequate stretching of tissue fibers which allowed for primary closure of a wound site.

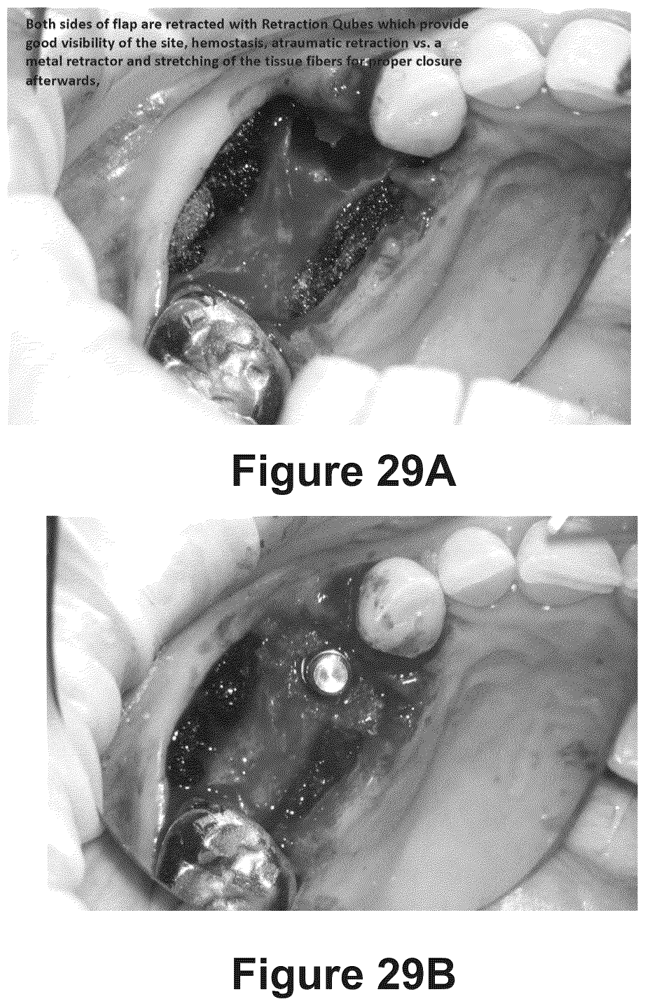

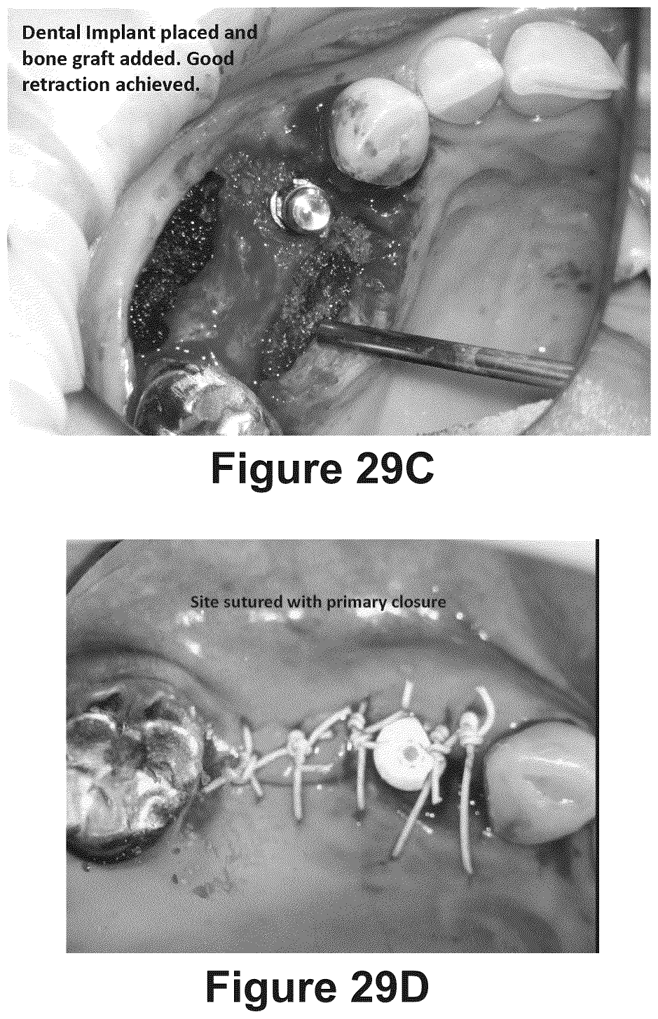

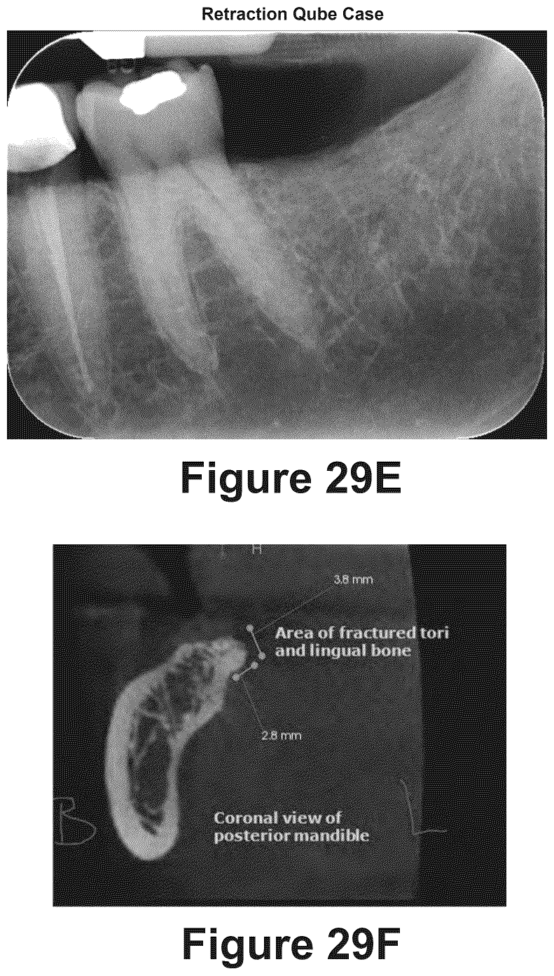

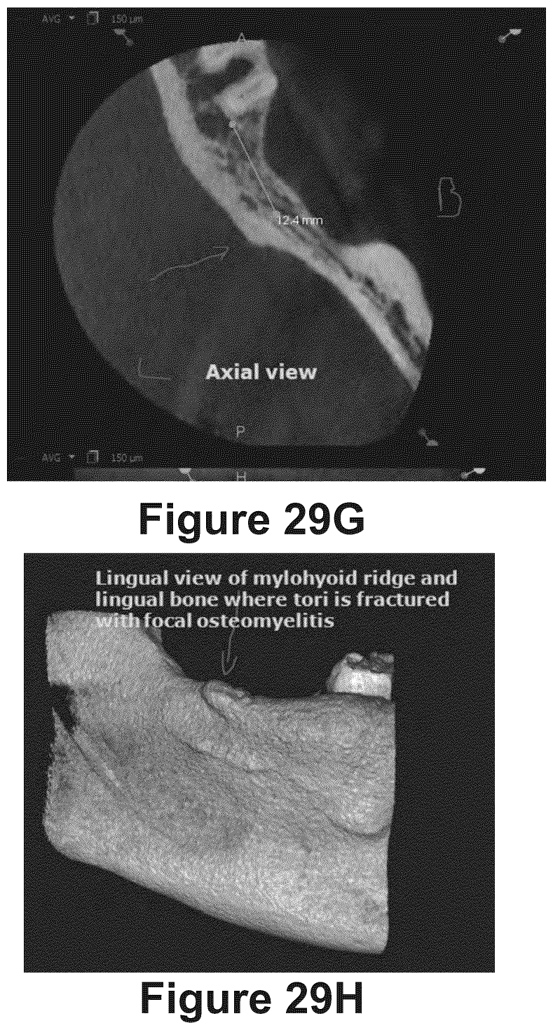

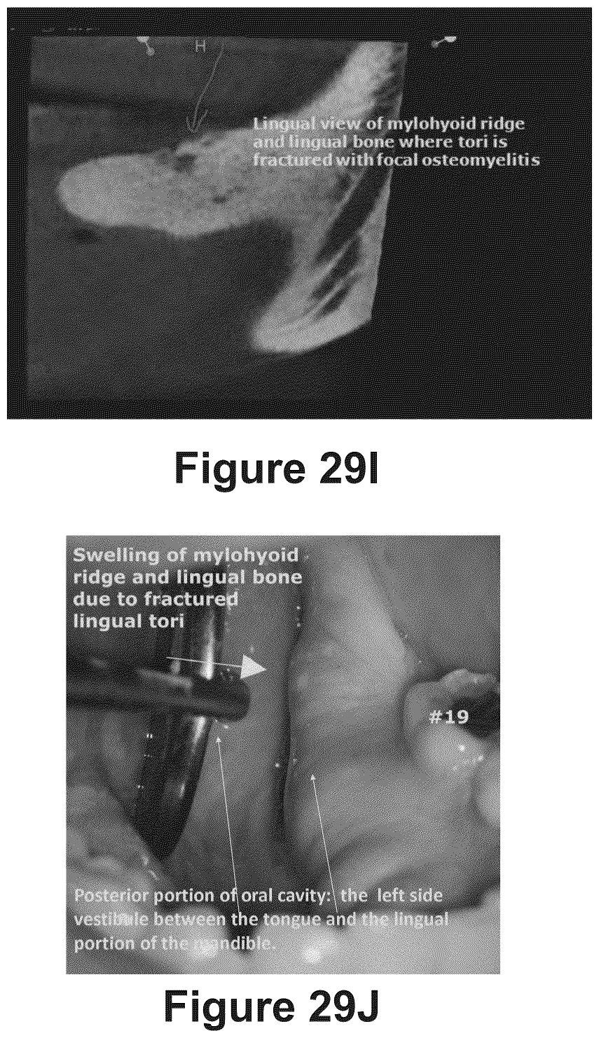

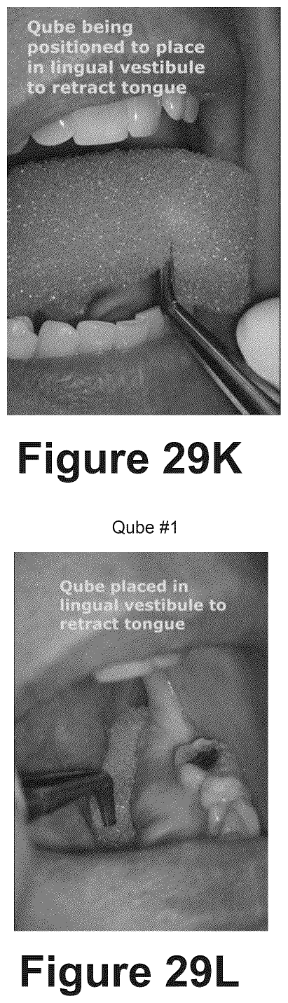

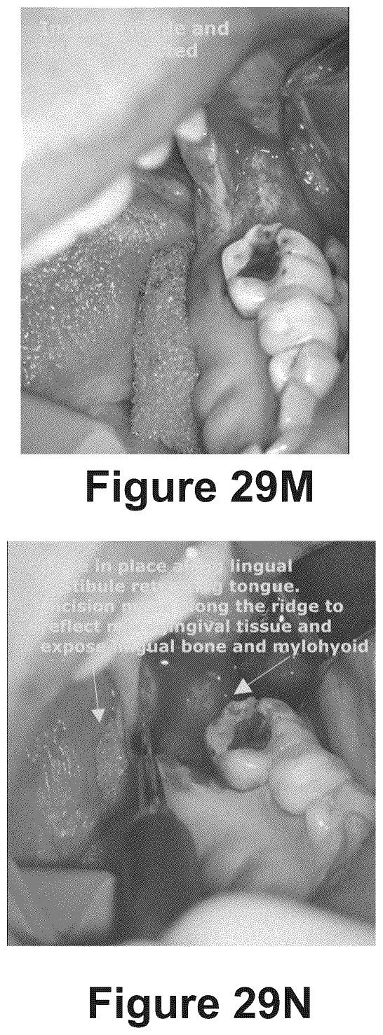

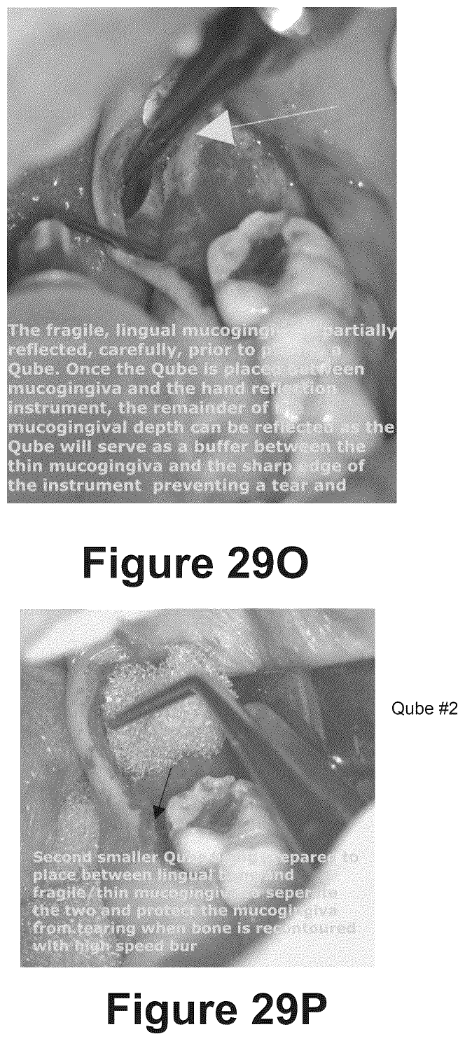

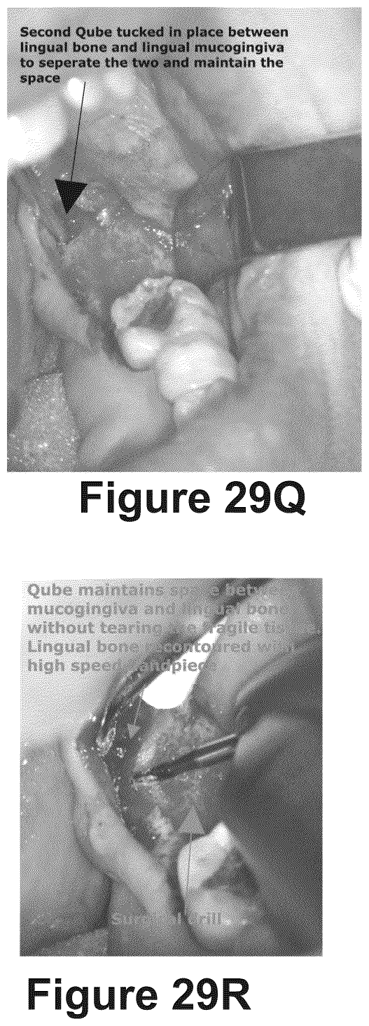

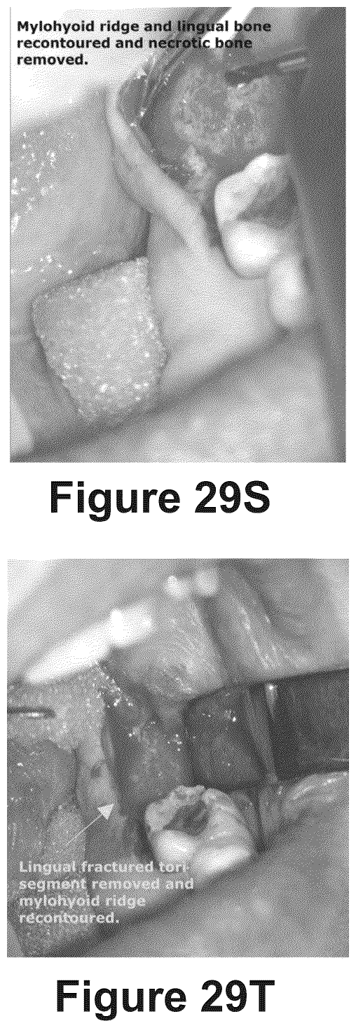

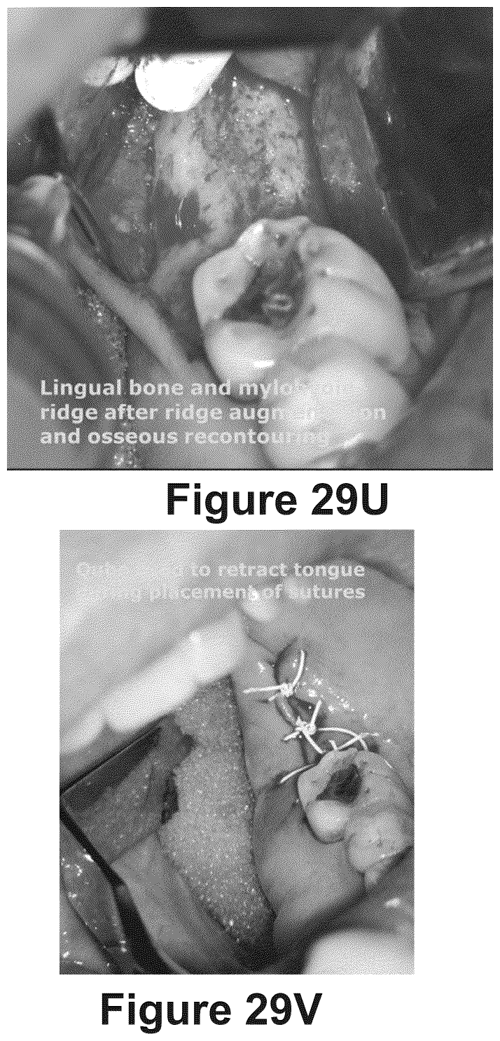

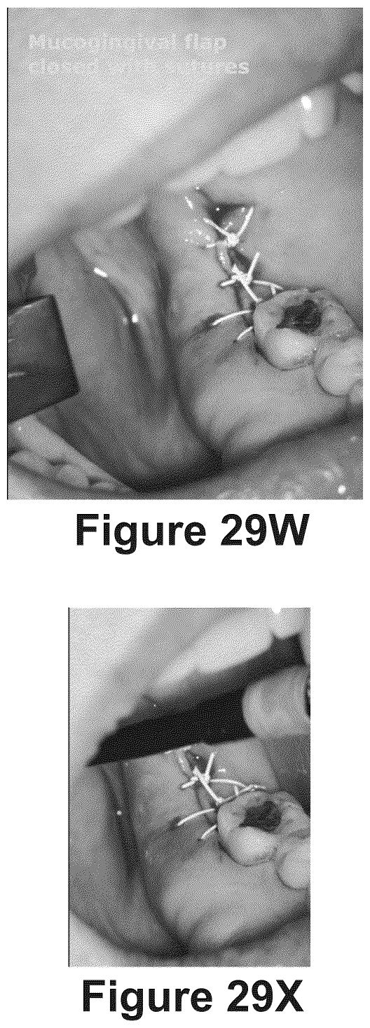

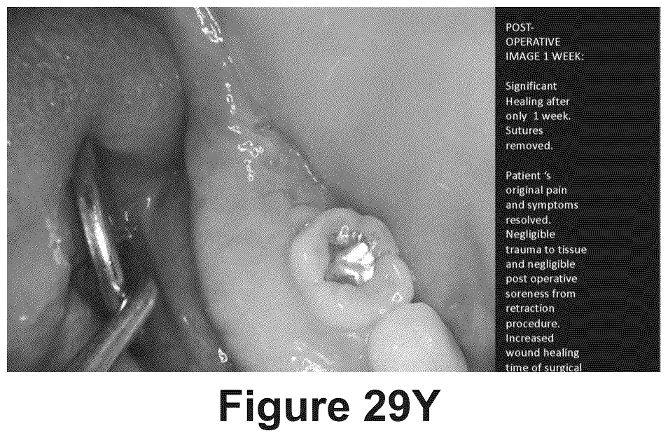

FIGS. 29A-29D show steps in multiple example oral surgical processes in accordance with an example embodiment.

FIG. 29A shows both sides of a flap being retracted with retraction qubes in accordance with example embodiments. Use of the retraction qubes in this way provides good visibility of the oral surgical site, hemostasis, and atraumatic retraction, as well as stretching of the tissue fibers for proper closure after the surgery is completed. In contrast, use of metal retractors product greater pain and pooling of saliva and blood reducing visibility of the oral surgical site.

FIGS. 29B-29C show that a dental implant has been placed and that a bone graft has been added. Good retraction had been achieved which contributed to a good result.

FIG. 29D shows the oral surgical site sutured with primary closure.

FIGS. 29E-29Y show another process in which a retraction qube may contribute to a positive result in an oral surgery.

FIG. 29F shows a coronal view of a posterior mandible. An area of a fractured tori and lingual bone are identified.

FIG. 29G shows an axial view of the same mouth region.

FIG. 29H shows a lingual view of a mylohyoid ridge and lingual bone where the tori is fractured with focal osteomyelitis.

FIG. 29I shows another lingual view of the mylohyoid ridge and lingual bone where the tori is fractured with focal osteomyelitis.

FIG. 29J shows swelling of mylohyoid ridge and lingual bone due to fractured lingual tori. Regarding the posterior portion of this oral cavity, the left side vestibule is shown between the tongue and the lingual portion of the mandible.

FIG. 29K shows an example of a tongue retraction qube being positioned to be placed in a lingual vestibule to retract the tongue.

FIG. 29L shows an example of a qube placed in a lingual vestibule to retract the tongue.

FIG. 29M shows an example of an incision made and tissue reflected.

FIG. 29N shows a qube in place along a lingual vestibule retracting the tongue. An incision has been made along the ridge to reflect mucogingival tissue and expose lingual bone and mylohyoid.

FIG. 29O shows a fragile, lingual mucogingiva that is partially reflected, carefully, prior to placing a qube. Once the qube is placed between mucogingiva and the hand reflection instrument, a remainder of the mucogingival depth can be reflected as the qube will serve as a buffer between the thin mucogingiva and the sharp edge of the instrument preventing a tear.

FIG. 29P shows a second qube, smaller than the first qube, being prepared to be placed between lingual bone and fragile/thin mucogingiva to separate the two and protect the mucogingiva from tearing when a bone is recontoured with a high speed bur.

FIG. 29Q shows the second qube tucked in place between lingual bone and lingual mucogingiva to separate the two and maintain the space.

FIG. 29R shows a qube maintaining space between mucogingiva and a lingual bone thereby preventing tearing of the fragile tissue. The lingual bone is recontoured with a high speed handpiece.

FIG. 29S illustrates a recontoured mylohyoid ridge and lingual bone. The necrotic bone has been removed.

FIG. 29T shows a lingual fractured tori segment removed and a mylohyoid ridge recontoured.

FIG. 29U shows a lingual bone and mylohyoid ridge after ridge augmentation and osseous recontouring.

FIG. 29V shows a qube being used to retract a tongue during placement of sutures.

FIG. 29W shows a mucogingival flap closed with sutures.

FIG. 29X shows another step in the process.

FIG. 29Y shows significant healing after only one week and the sutures are removed. The patient's original pain and symptoms have been resolved. Negligible trauma is apparent to tissue and negligible post operative soreness is apparent from the retraction procedure.

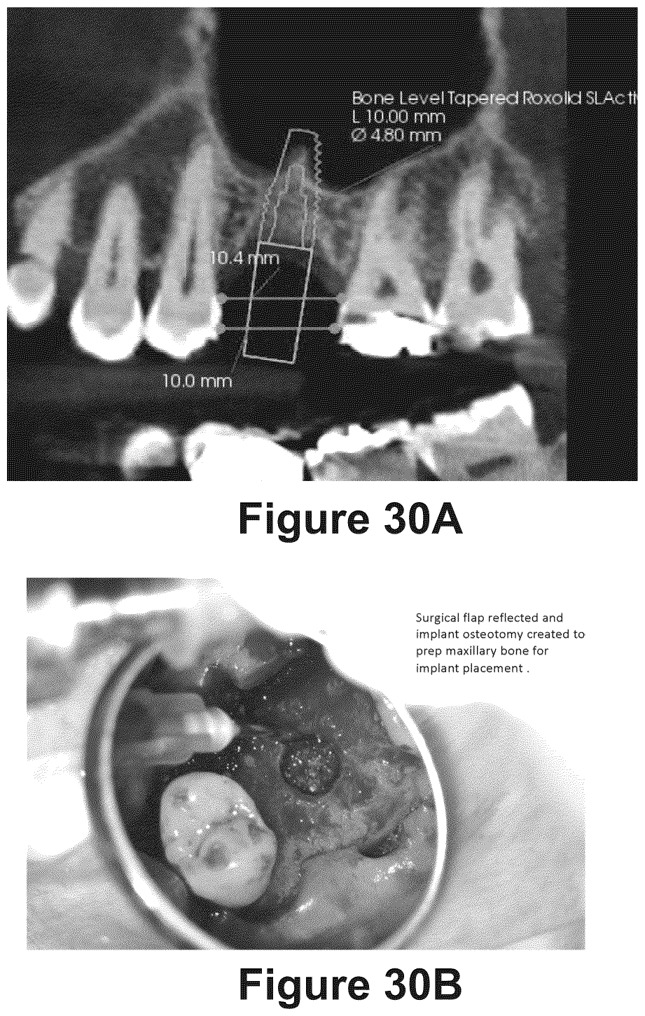

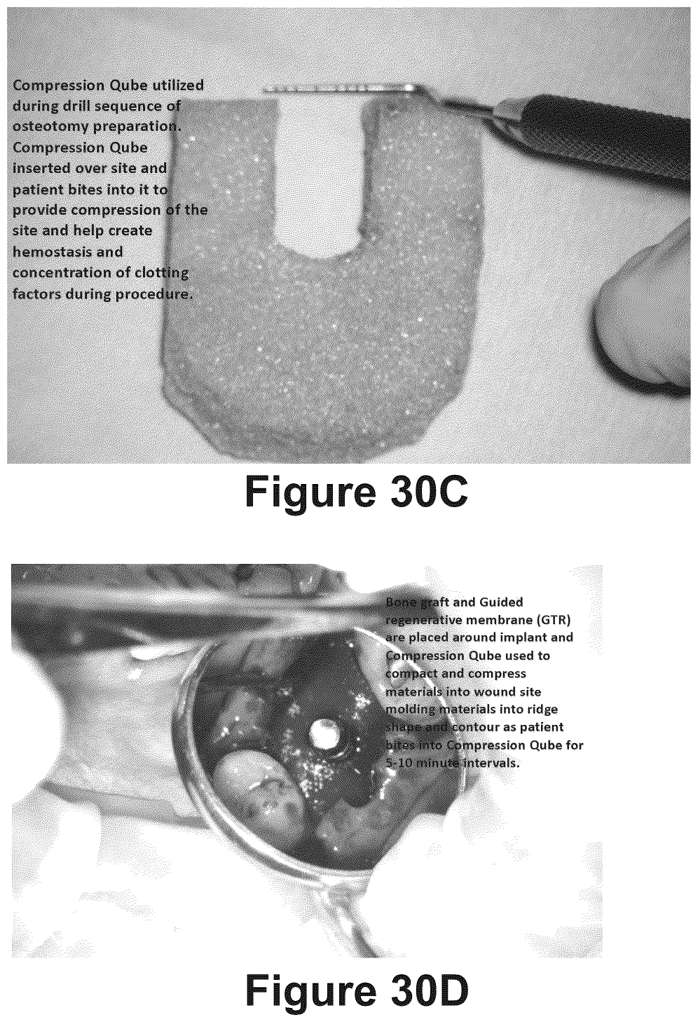

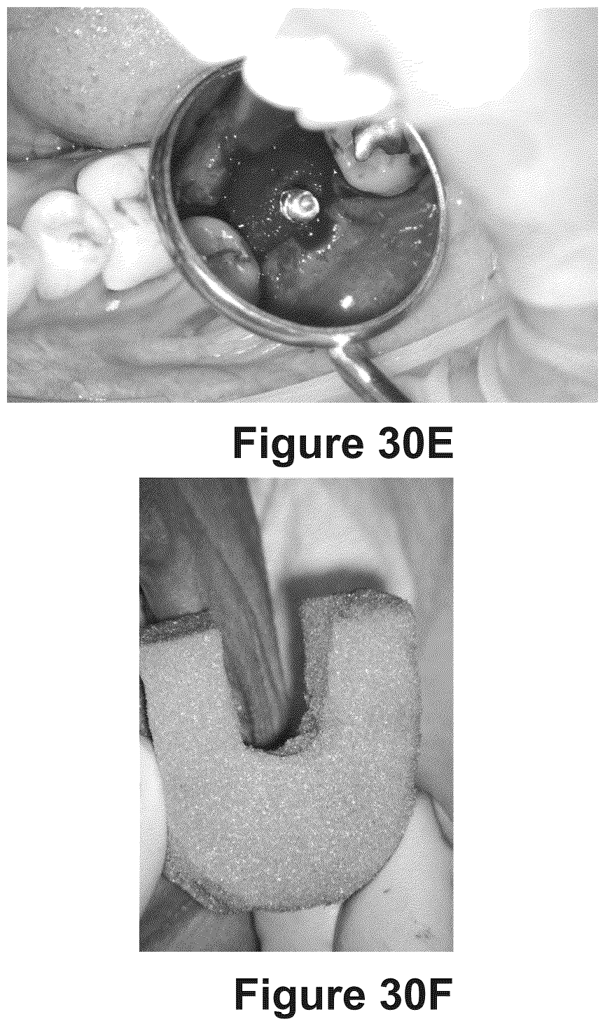

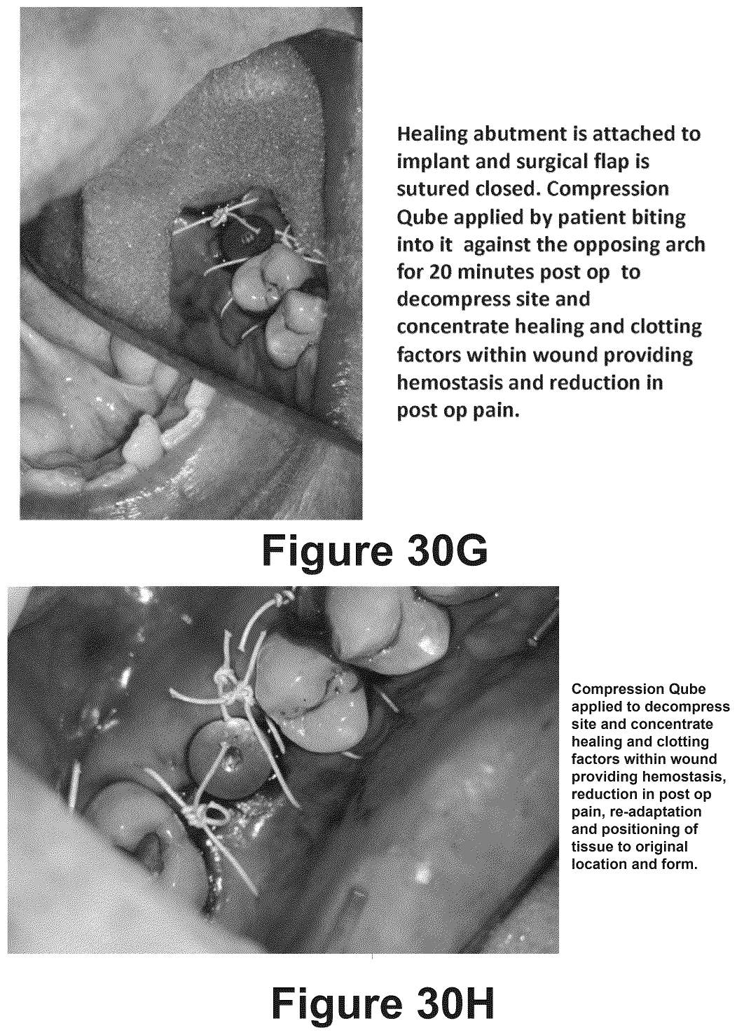

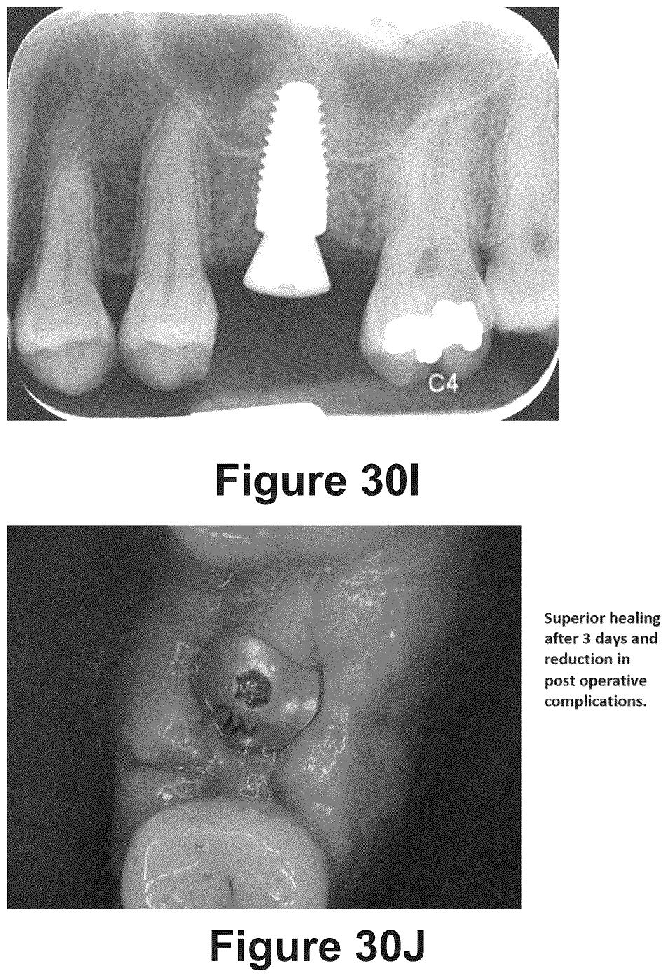

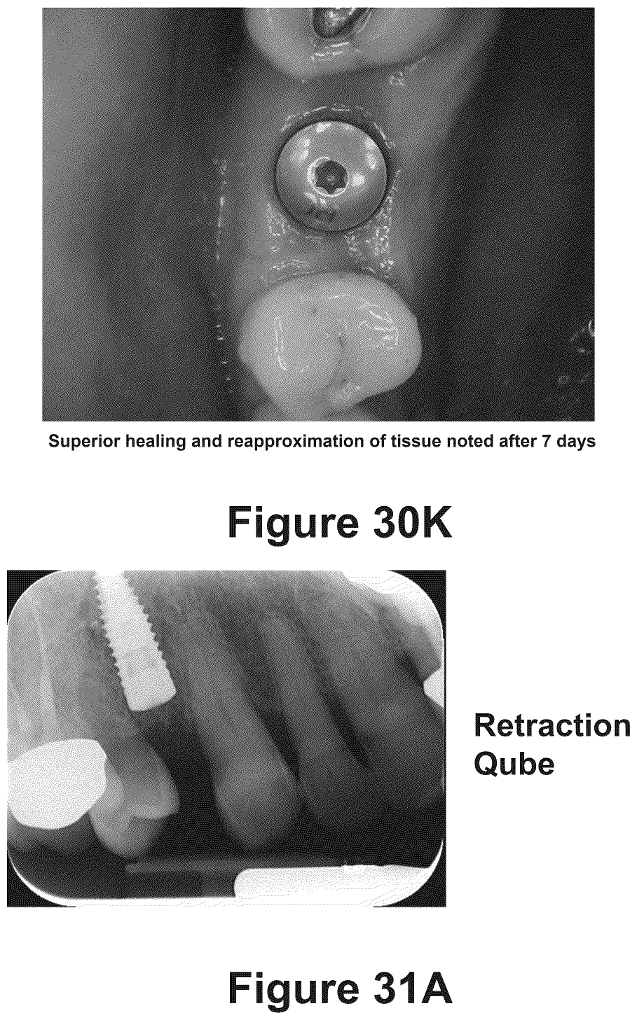

FIGS. 30A-30K show steps in an oral surgical implant placement process in accordance with an example embodiment.

FIG. 30B shows a surgical flap reflected and an implant osteotomy created to prepare a maxillary bone for implant placement.

FIG. 30C shows a compression qube, aka a C-series qube or a C qube, to be utilized during a drill sequence of osteotomy preparation. The compression qube is to be inserted over the oral surgical site such as to enable the patient to bite into the compression qube. The advantageous qube material, shape and size provide compression of the site when the patient bites down. This compression at the wound site helps create hemostasis and a concentration of clotting factors during an oral surgical procedure.

FIG. 30D shows a bone graft and a guided regenerative membrane, aka GTR, that are placed around an implant. A compression qube is used to compact and compress materials into a wound site to mold materials into ridge shape and contour as patient bites down into the compression qube for 5-10 minute intervals.

FIG. 30E shows an implant in place at an oral surgical site.

FIG. 30F shows a U-shaped compression qube, or C-series qube or C qube, in accordance with an example embodiment prior to placement and compression at the oral surgical site shown in FIG. 30E.

FIG. 30G shows that a healing abutment is now attached to the implant, and the surgical flap is sutured closed. A compression qube is applied by patient biting into it against an opposing arch to 20 minutes post op to decompress the site and concentrate healing and clotting factors within the wound providing hemostasis and reduction in post-operative pain.

FIG. 30H shows a compression qube applied to decompress an oral surgical psite and concentrate healing and clotting factors within the wound providing hemostasis, reduction in post op pain, re-adaptation and positioning of tissue to original location and form.

FIG. 30I shows an x-ray of a side view of the implant placed at the location of an extracted tooth.

FIG. 30J shows superior healing after only 3 days. A reduction in post-operative complications is also apparent.

FIG. 30K shows superior healing and reapproximation of tissue noted after 7 days since placement of the implant.

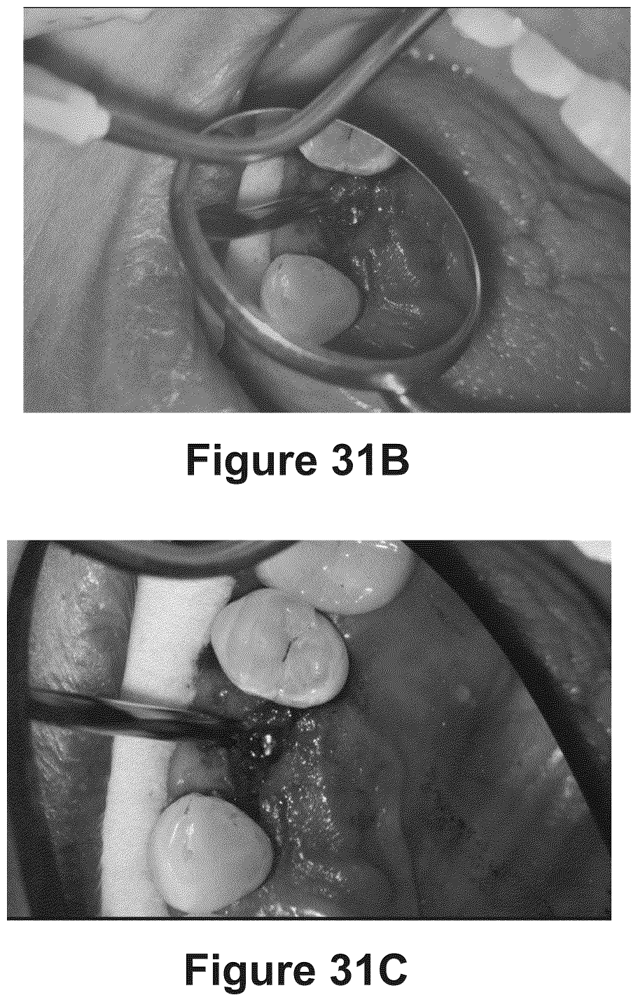

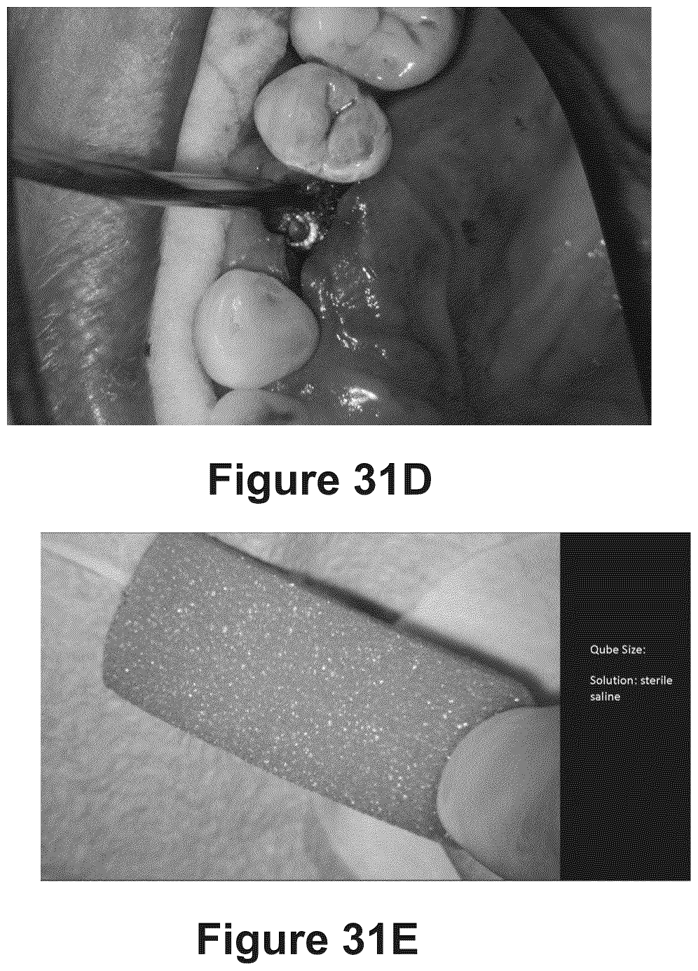

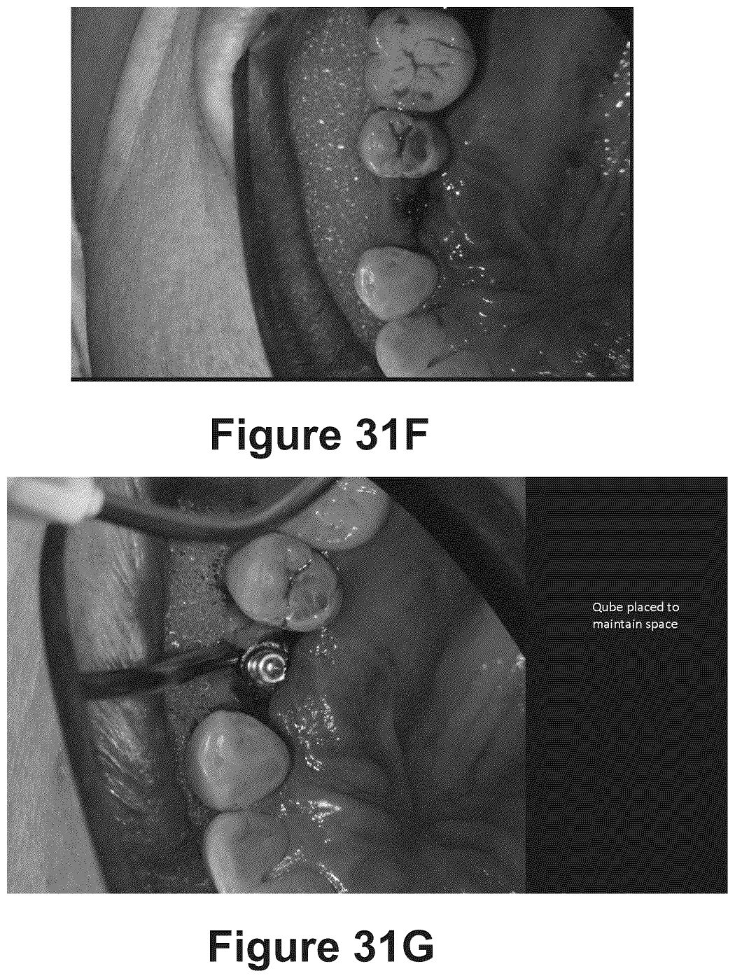

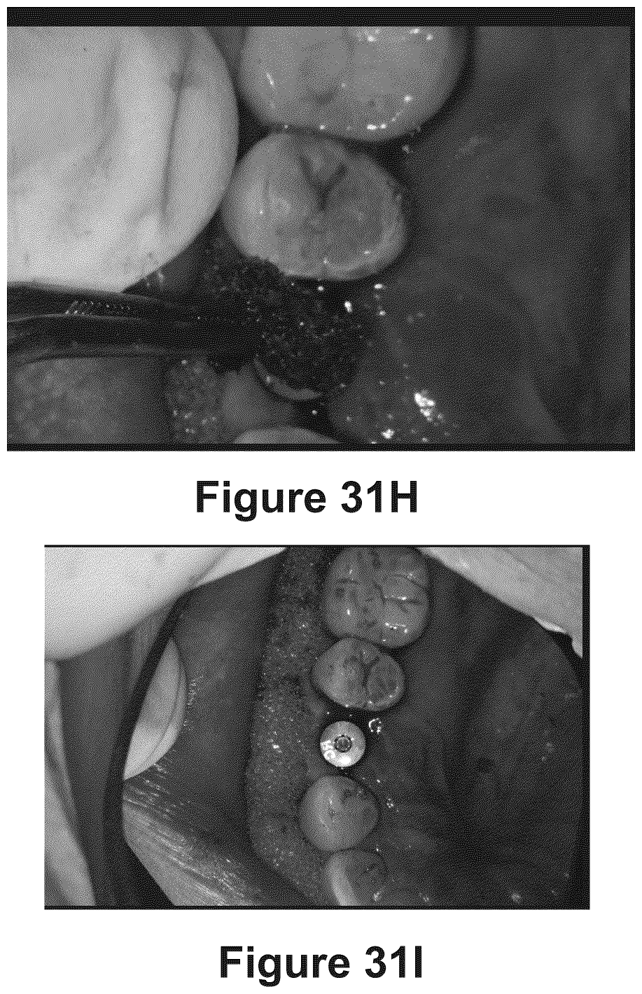

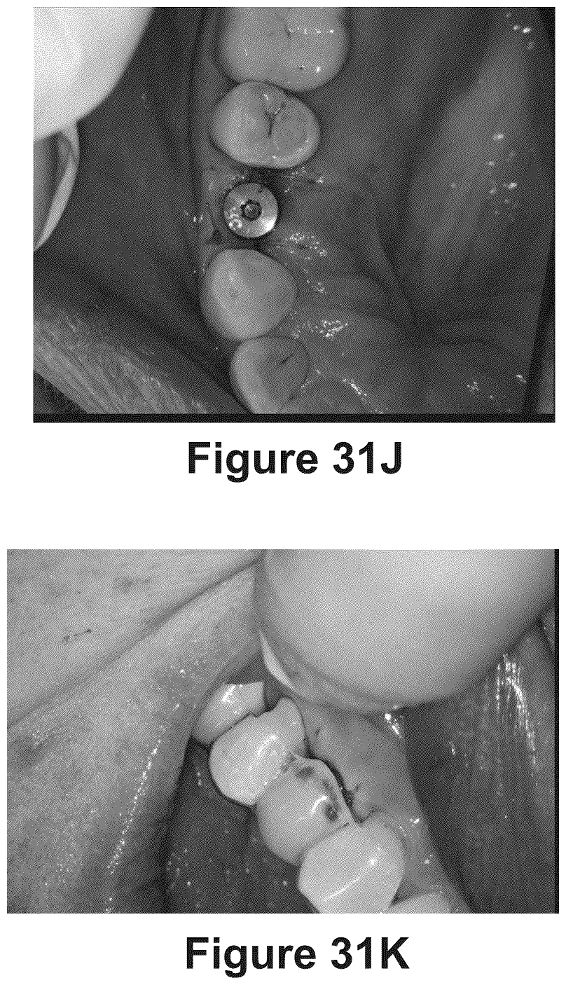

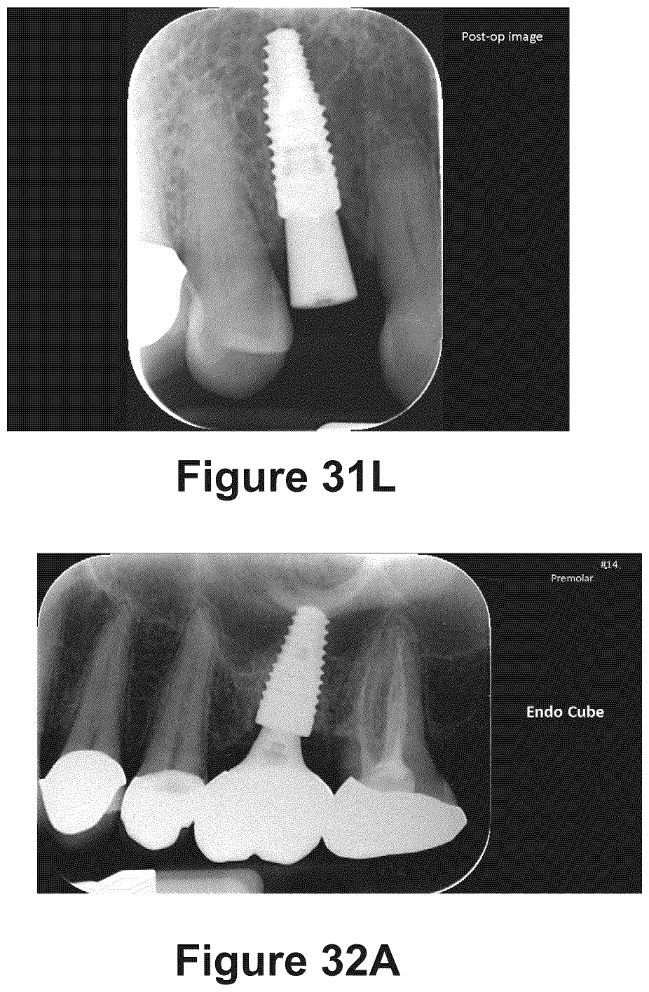

FIGS. 31A-31L illustrate steps in an oral surgical process involving a further use of a retraction qube in accordance with an example embodiment.

FIG. 31B shows a dental implant site.

FIG. 31C clearly shows the dental implant firmly in place at the site.

FIG. 31D shows another step in the oral surgical process.

FIG. 31E shows a retraction qube that is carrying a sterile saline solution.

FIG. 31F shows the retraction qube of FIG. 31E in its place proximate to the dental implant.

FIG. 31G shows a qube placed to maintain space around the dental implant.

FIG. 31H shows another step in the oral surgery.

FIG. 31I shows a qube and a dental implant at the site of an extracted tooth.

FIG. 31J shows the dental implant from another view.

FIG. 31K shows an abutment coupled to the dental implant shown in FIGS. 31A-31J.

FIG. 31L shows a post operative image of the oral surgical site.

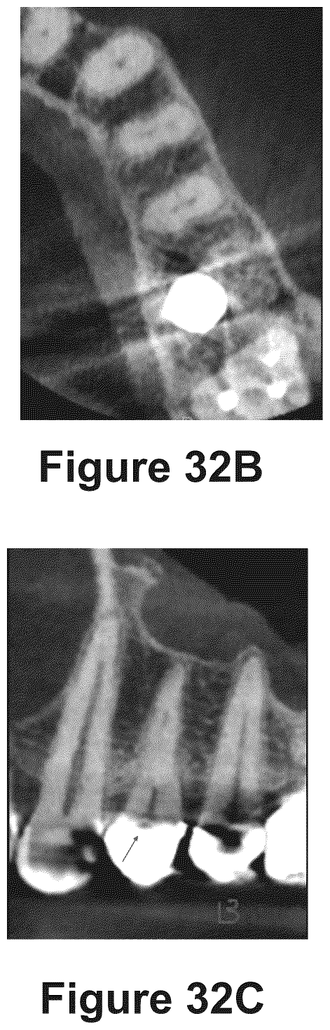

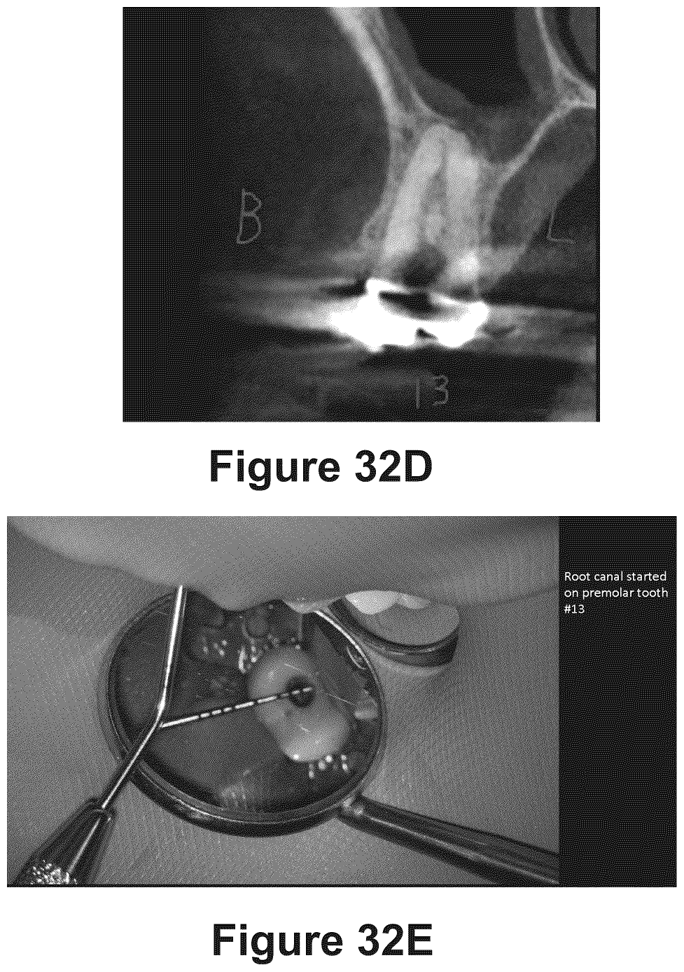

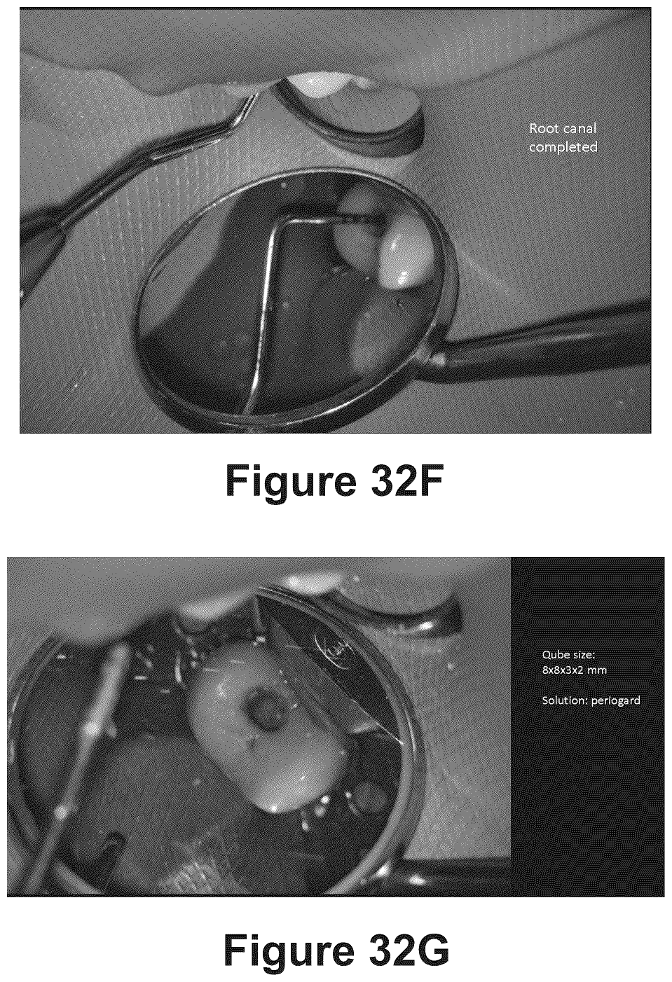

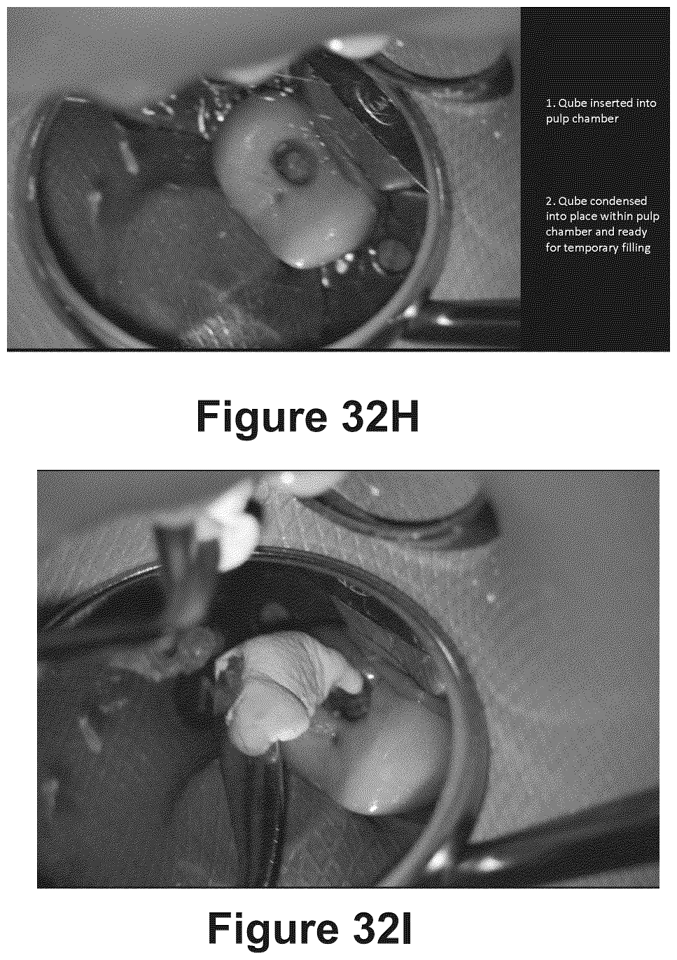

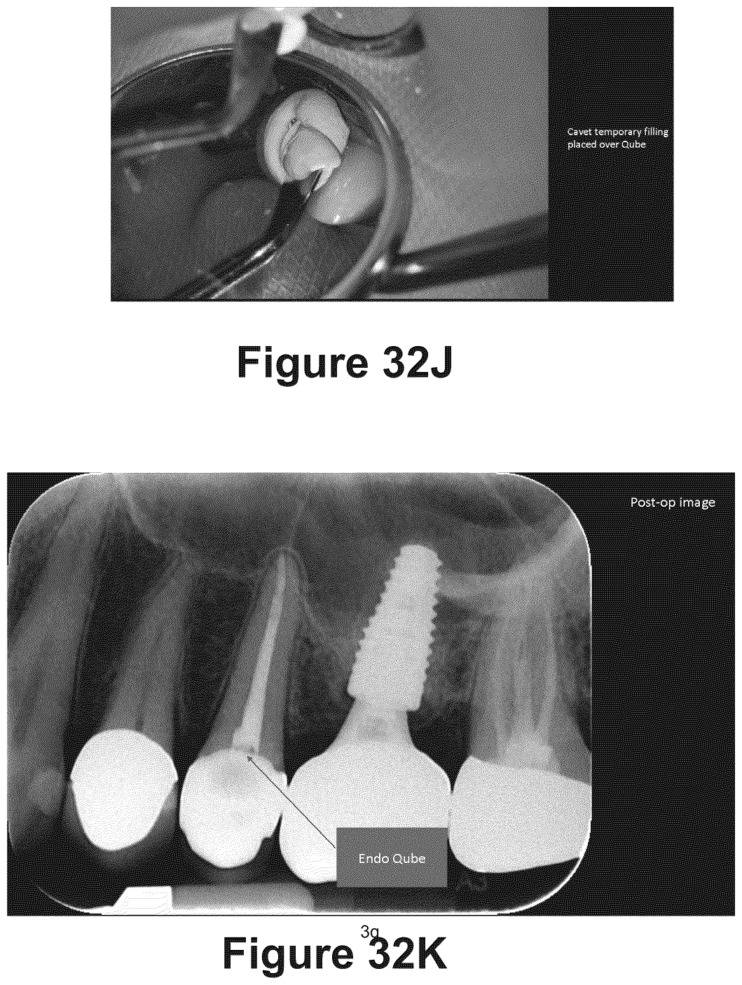

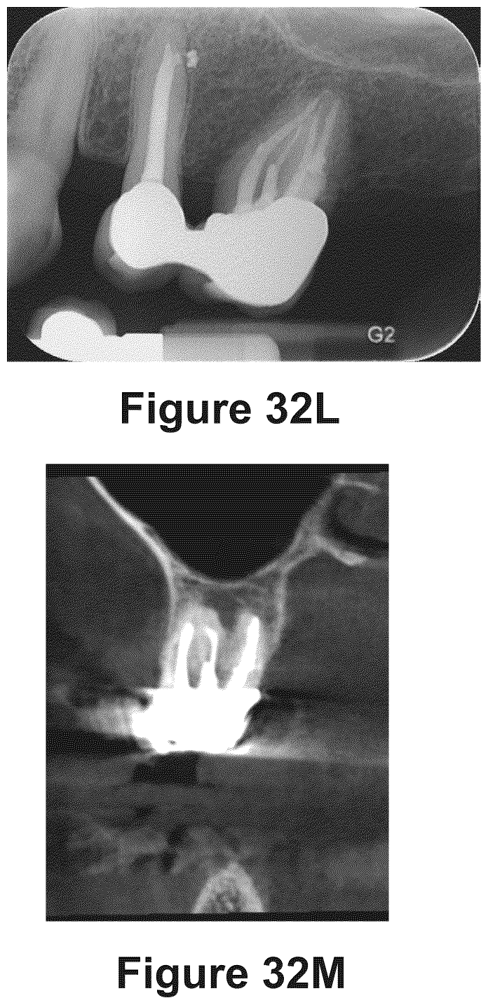

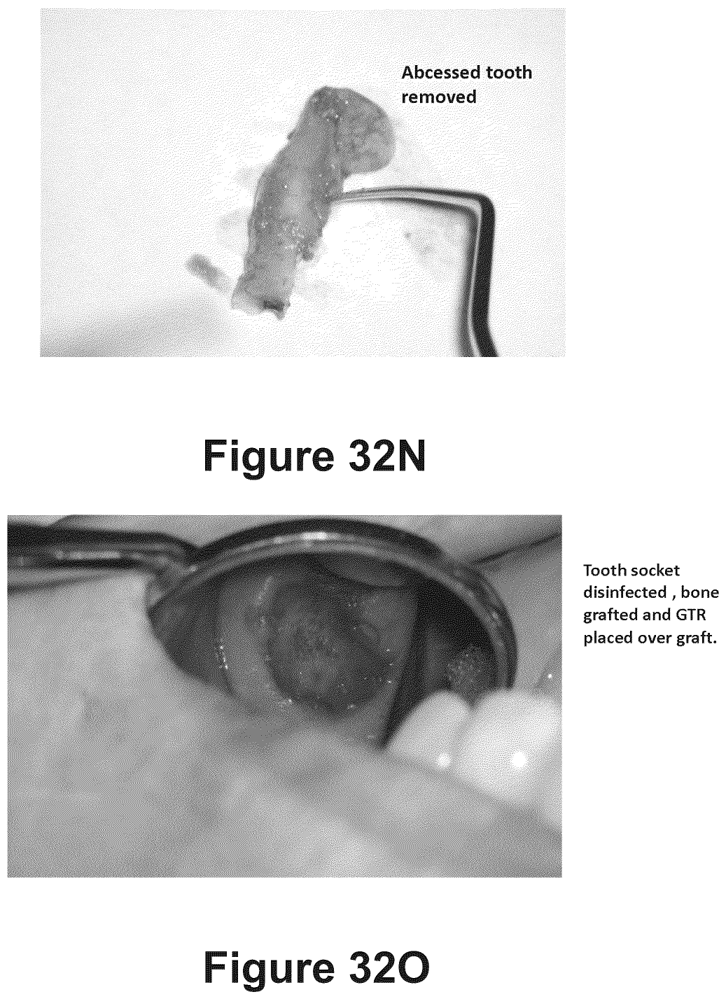

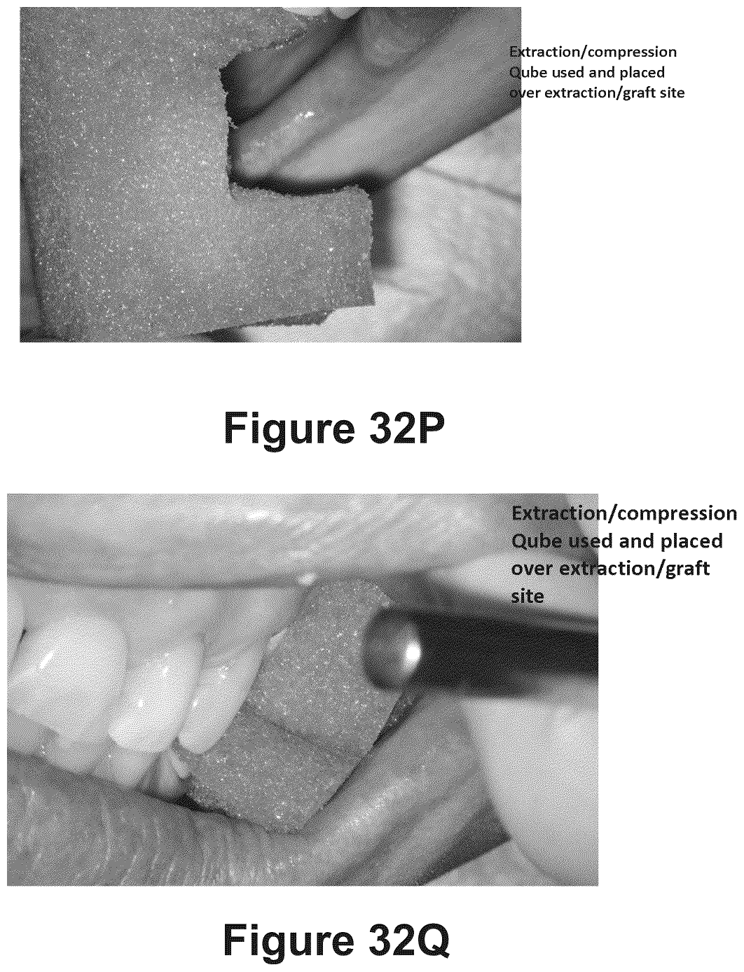

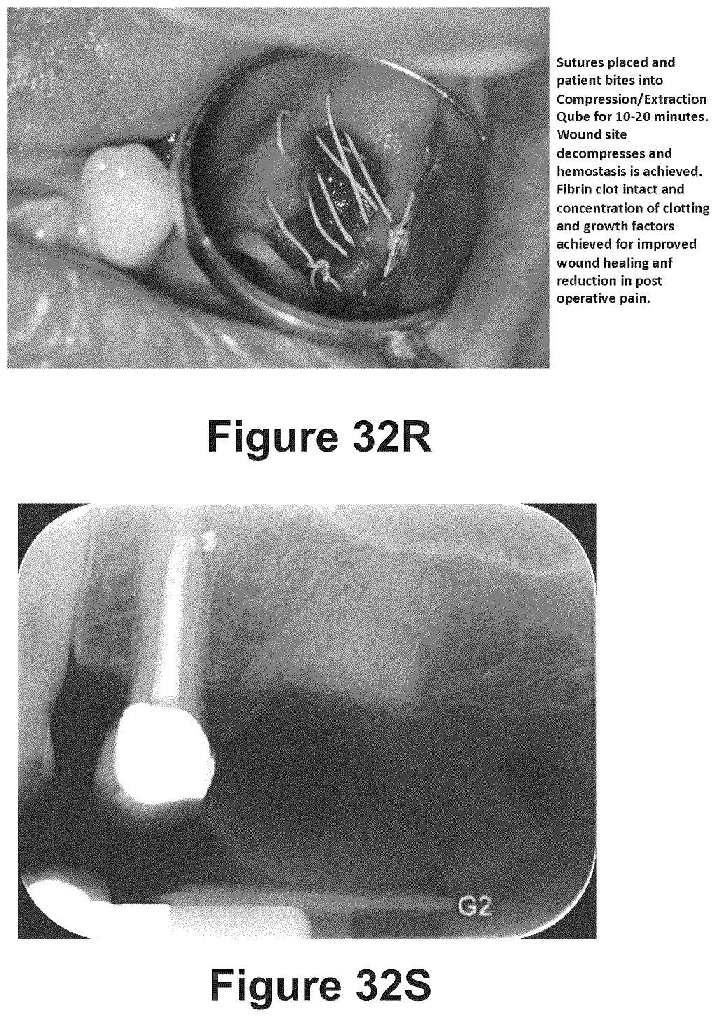

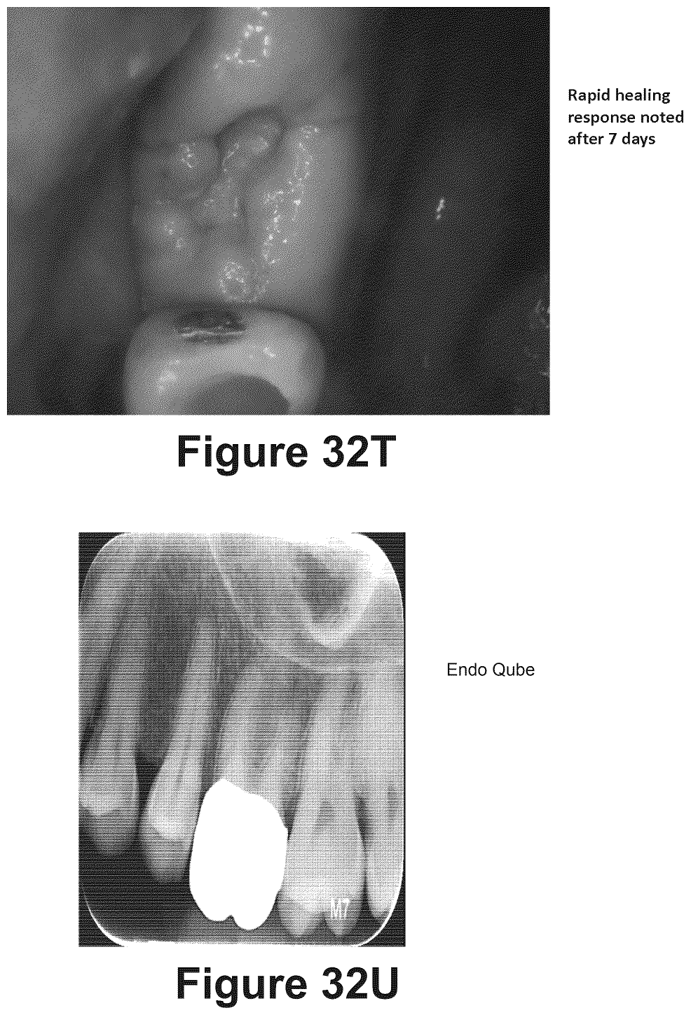

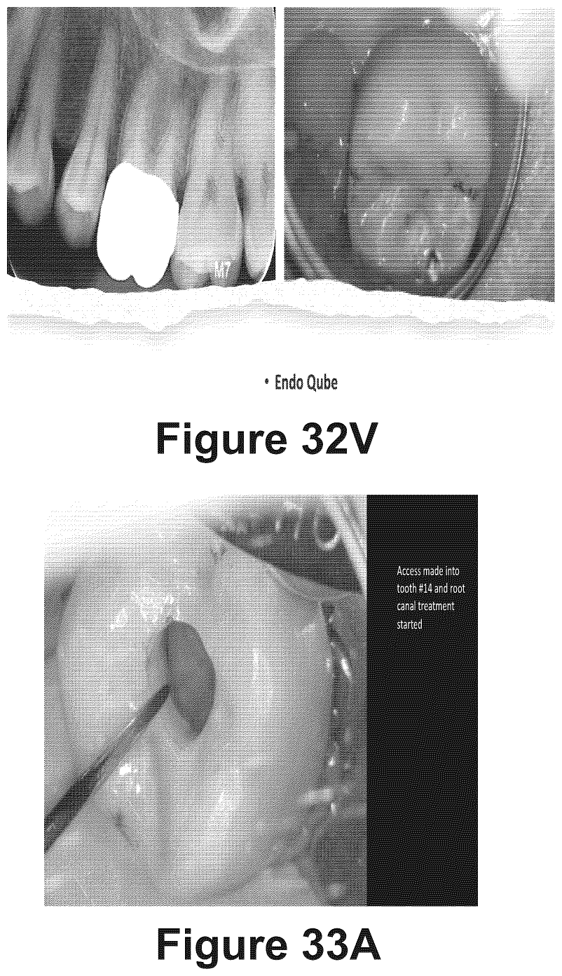

FIGS. 32A-32V illustrate an example oral surgical process involving an endo qube.

FIG. 32A is a side view x-ray.

FIG. 32B shows the same mouth region with the view rotated by 90 degrees from that shown in FIG. 32A.

FIG. 32C shows another view of the same mouth region.

FIG. 32D shows another view of the same mouth region.

FIG. 32E shows a start of a root canal on a premolar tooth.

FIG. 32F shows a completed root canal.

FIG. 32G shows a periogard solution using a qube that is 8×8×3×2 mm.

FIG. 32H shows a qube inserted into a pulp chamber of a tooth. The qube condenses into place within the pulp chamber which is ready for temporary filling.

FIG. 32I shows a next step in the oral surgical process.

FIG. 32J shows a caveat temporary filling placed over the qube.

FIG. 32K is a post operative side view x-ray image showing an endo qube within the cavity beneath the temporary filling.

FIGS. 32L-32M show additional images of the oral surgical site.

FIG. 32N shows an abscessed tooth that has been removed.

FIG. 32O shows a tooth socket disinfected, bone grafted, and a GTR placed over the graft material.

FIG. 32P shows an extraction/compression qube used and placed over the extraction/graft site.

FIG. 32Q shows an extraction/compression qube used and placed over an extraction/graft site.

FIG. 32R shows sutures placed and patient bites into compression/extraction qube for 10-20 minutes. The wound site decompresses and hemostasis is achieved. Fibrin clot intact and concentration of clotting and growth factors achieved for improved wound healing and reduction in post operative pain.

FIG. 32S shows a post-operative x-ray of the site where the abscessed tooth had been extracted.

FIG. 32T shows a rapid healing response after 7 days.

FIG. 32U illustrates an example of an endo qube.

FIG. 32V illustrate examples of endo qubes.

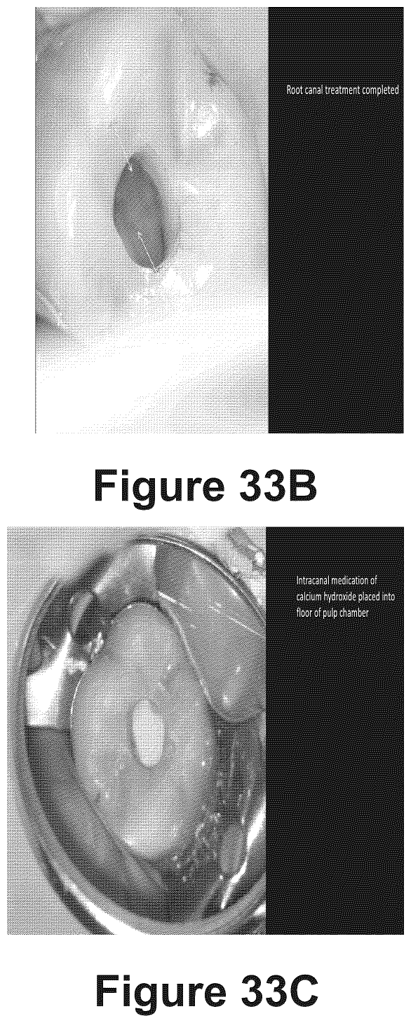

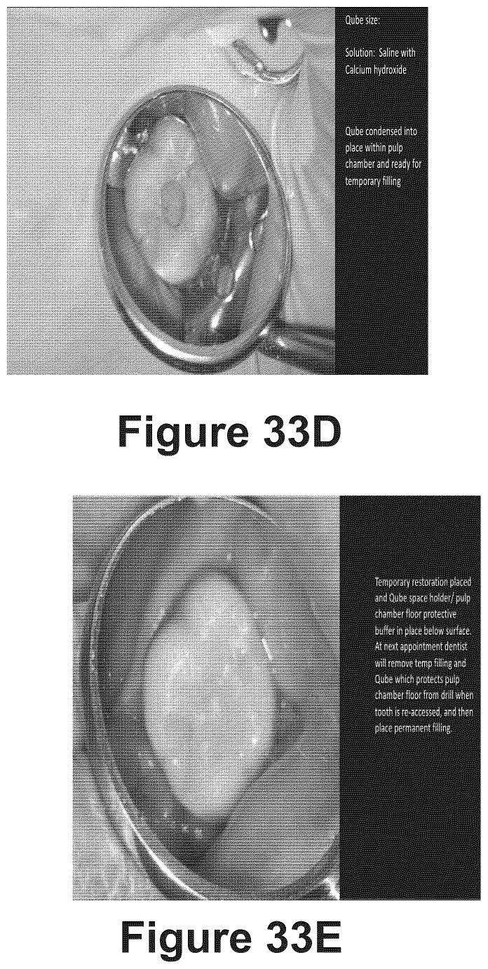

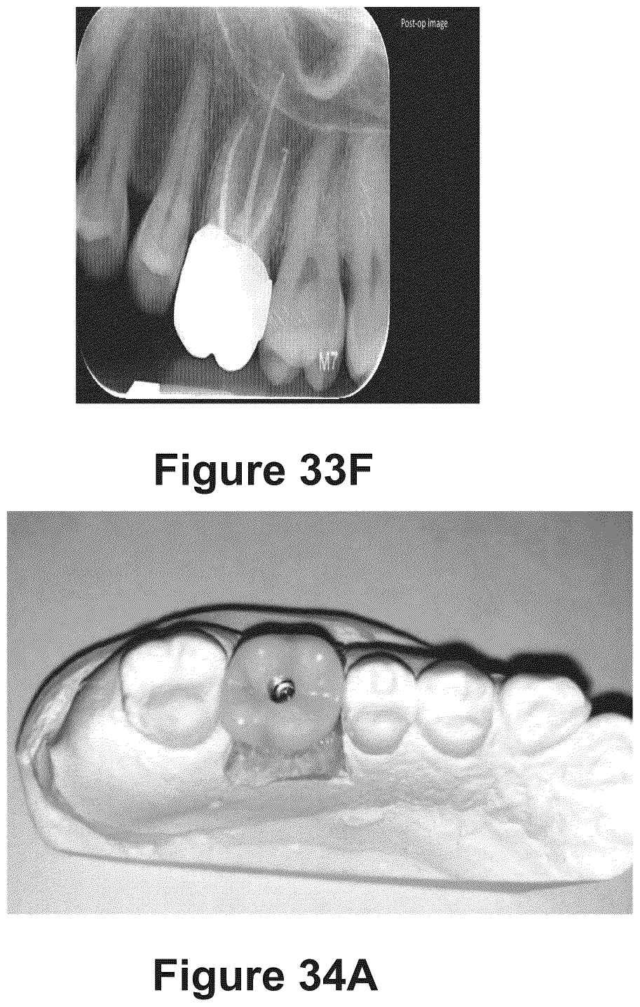

FIGS. 33A-33F illustrate an example process that involves use of one or more endo qubes.

FIG. 33A shows an x-ray.

FIG. 33B shows the x-ray of FIG. 33A alongside a top view of an oral surgical site.

FIG. 33C shows that an access has been made into a tooth and that a root canal has been started.

FIG. 33D shows that a root canal has been completed.

FIG. 33E shows that an intracranial medication of calcium hydroxide has been placed into a floor of a pulp chamber.

FIG. 33F shows a qube condensed into place within the pulp chamber which is ready for temporary filling.

FIG. 33G shows that a temporary restoration has been placed and a qube space holder has established a pulp chamber floor protective buffer in place below the surface. At a subsequent appointment, a dentist may remove the temporary filling and qube which protects the pulp chamber floor from a drill when the tooth is re-accessed. Then, a permanent filling can be placed.

FIG. 33H shows a post-operative image.

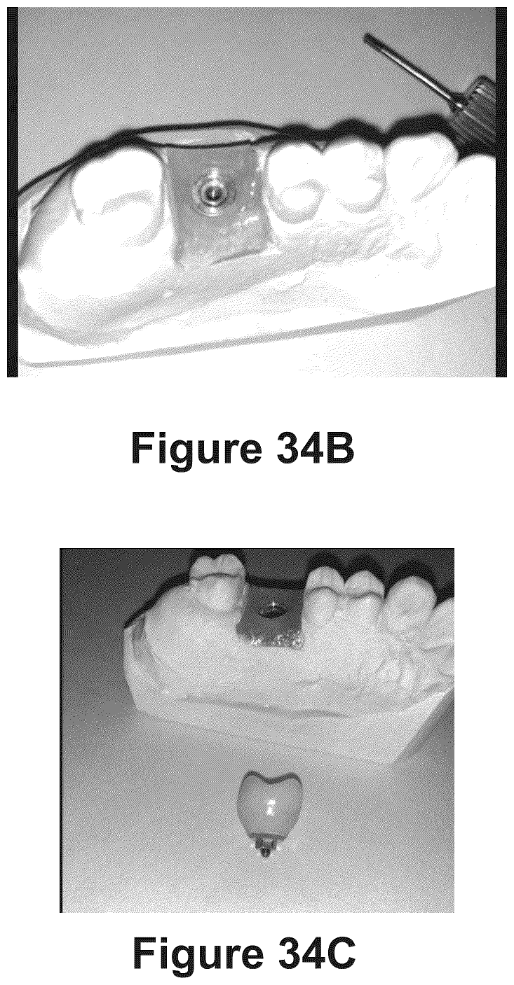

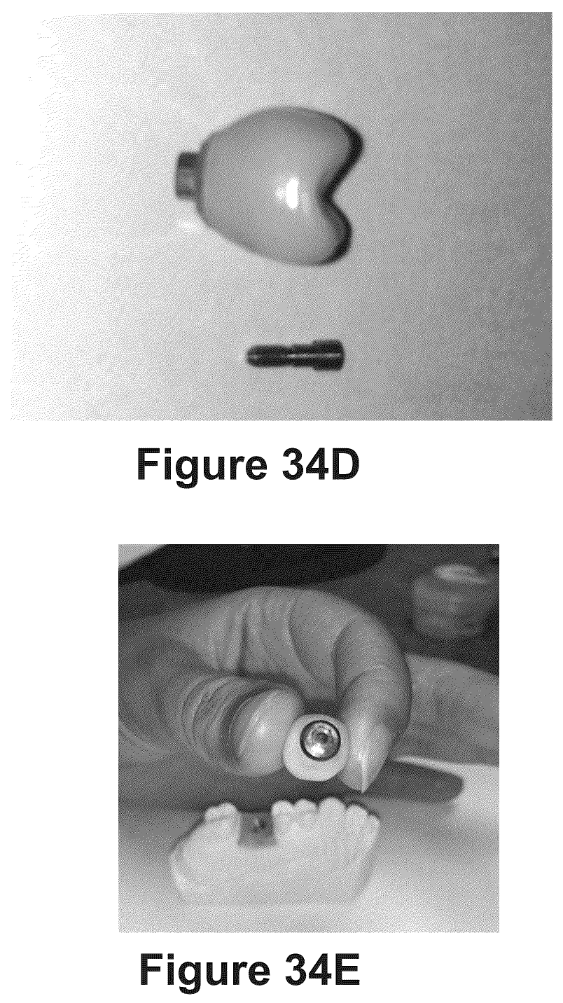

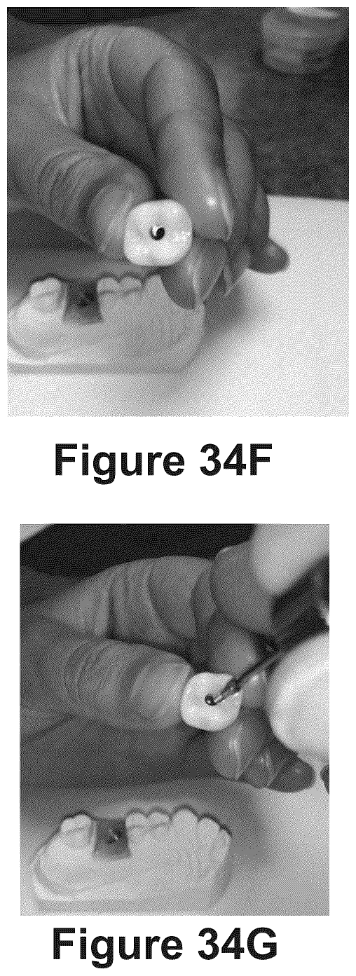

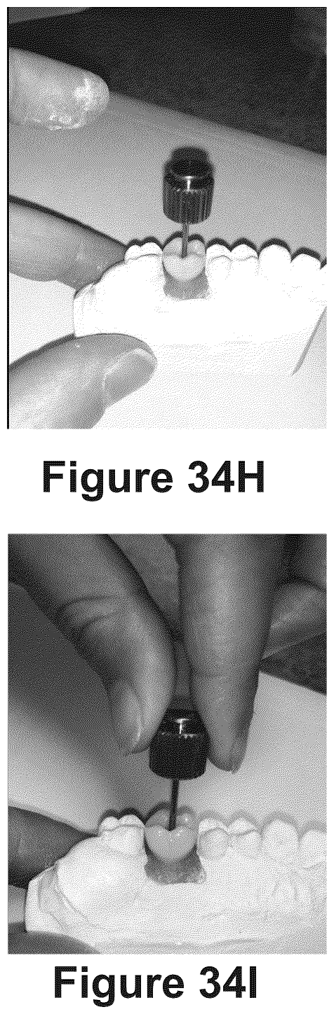

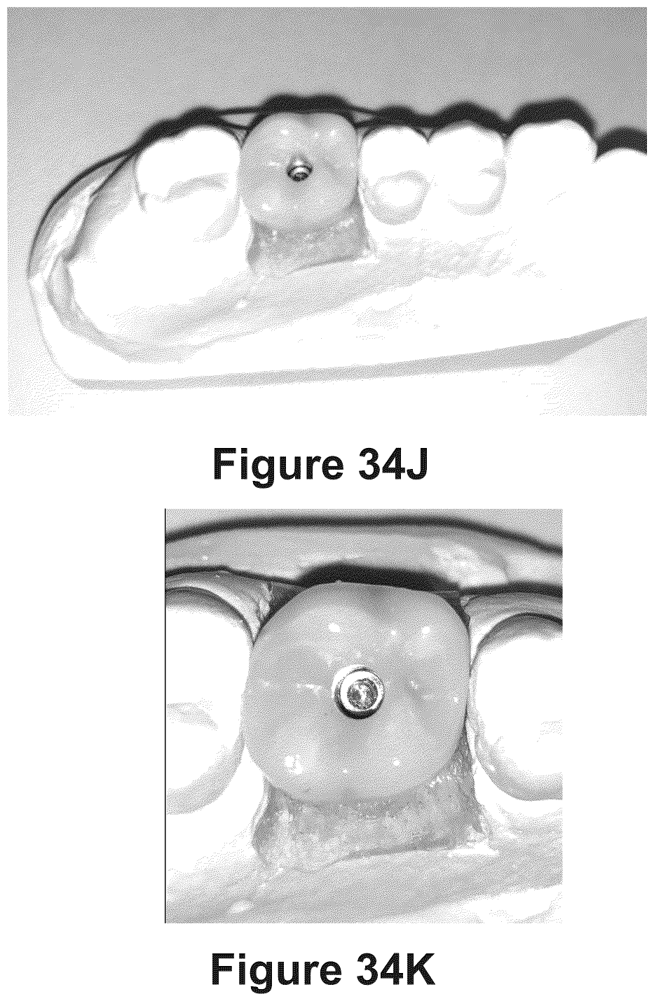

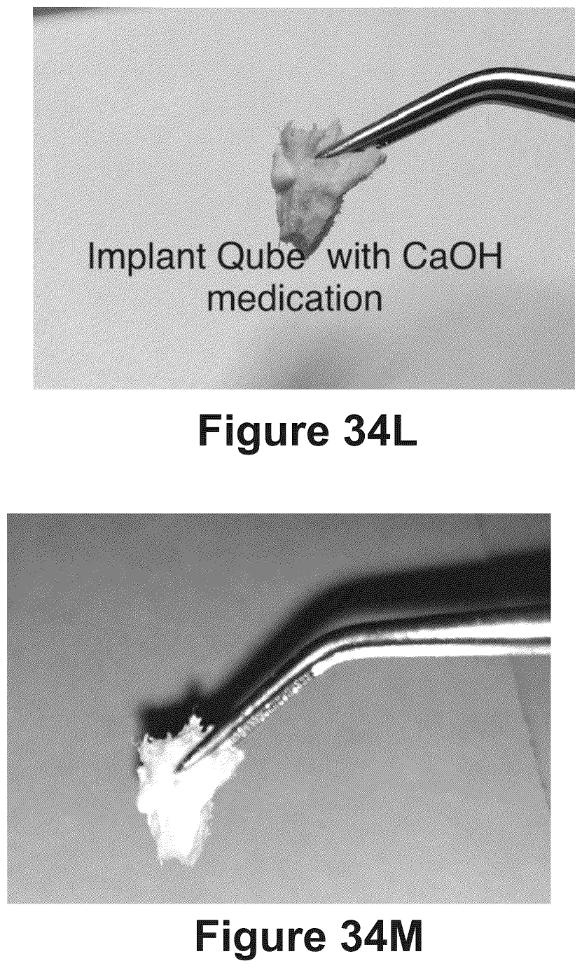

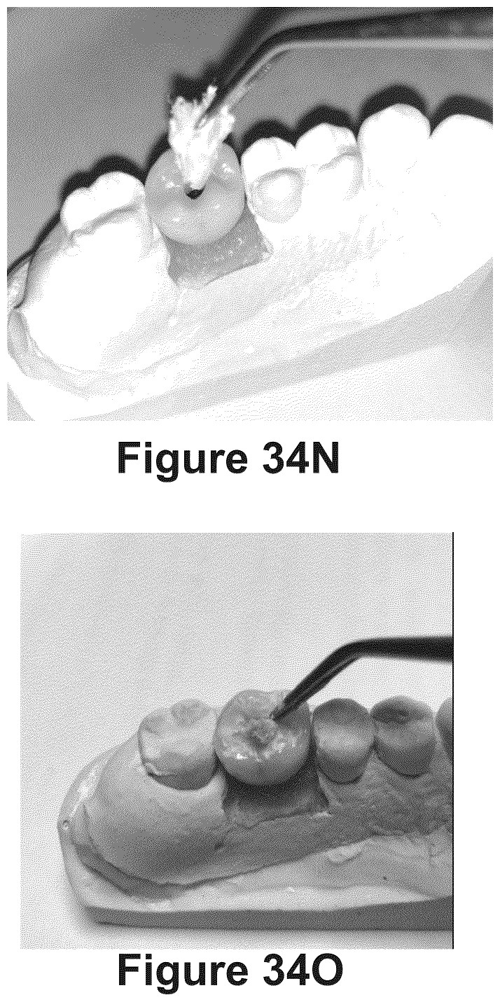

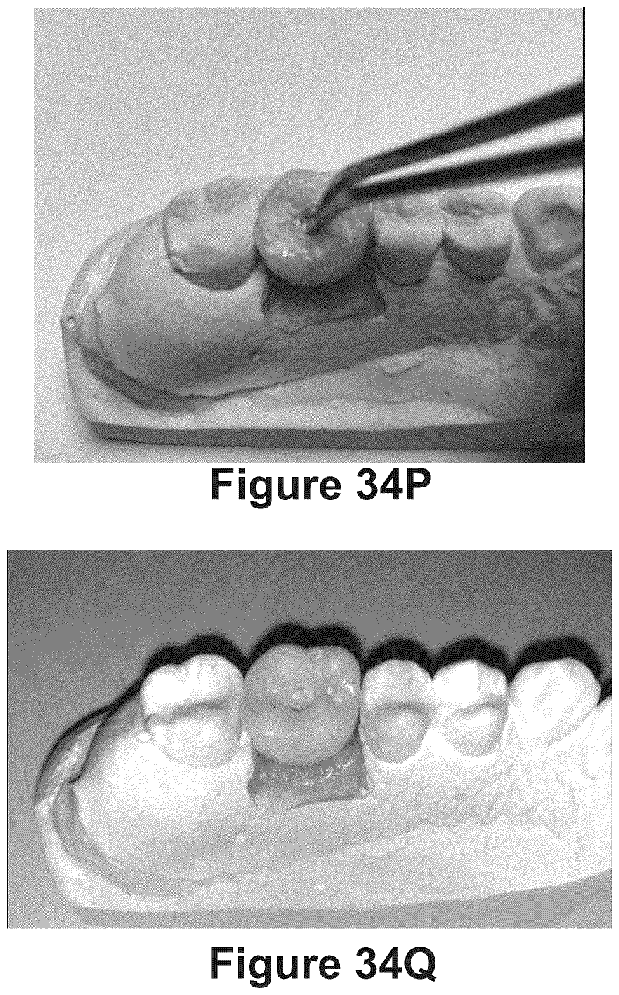

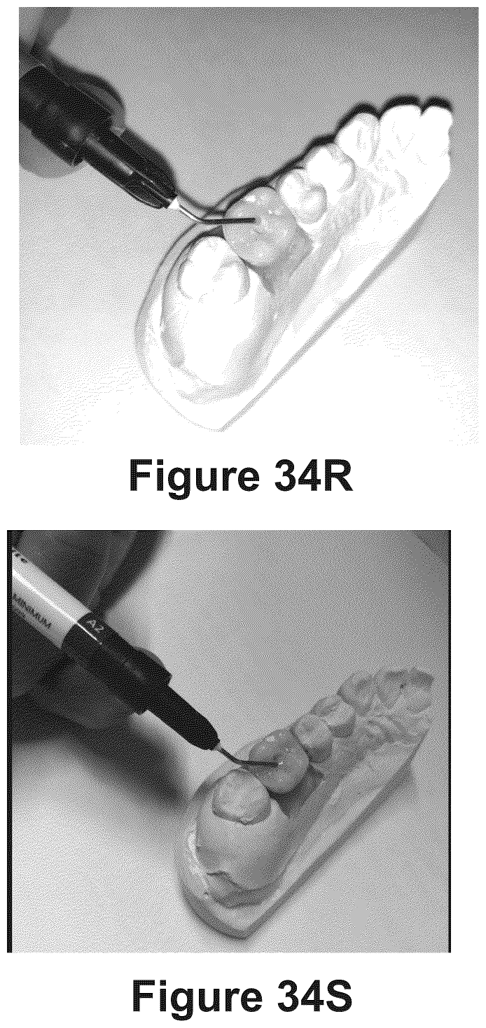

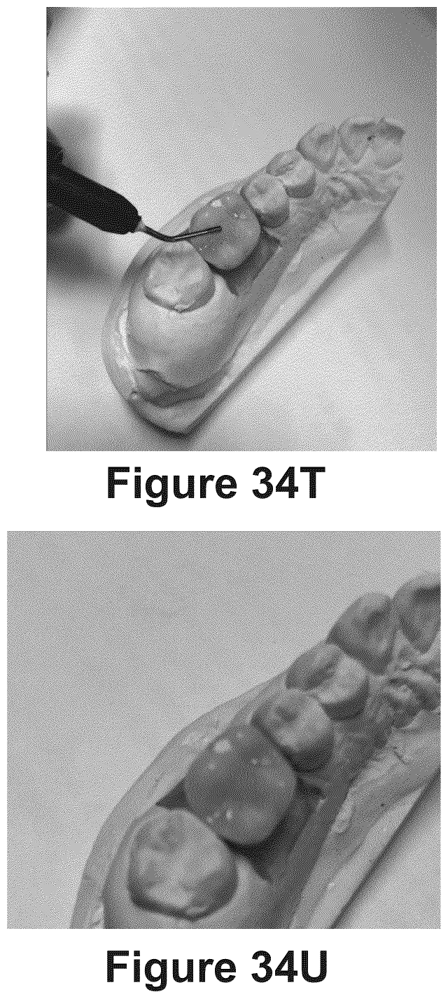

FIGS. 34A-34U illustrate a dental implant process following a tooth extraction that involves use of an implant qube placed in a recess that is medicated with CaOH.

FIGS. 34A-34H illustrate an example abutment-to-implant coupling step using an abutment coupling tool. An abutment may be coupled to an implant that includes interior screw threads by tightening a screw through a cylindrical passage in the abutment using the abutment coupling tool, which may include an Allen wrench, or a Phillips or flathead screwdriver configuration or the like.

FIGS. 341-34K illustrate a dental implant abutment coupled securely to a dental implant in accordance with example embodiments.

FIGS. 34L-34N illustrate an example step wherein an implant qube with or

without a medicament such as CaOH in accordance with example embodiments may be placed within the cylindrical passage in the abutment.

FIGS. 34O-34U illustrate filling the cylindrical passage in the abutment in accordance with an example embodiment.

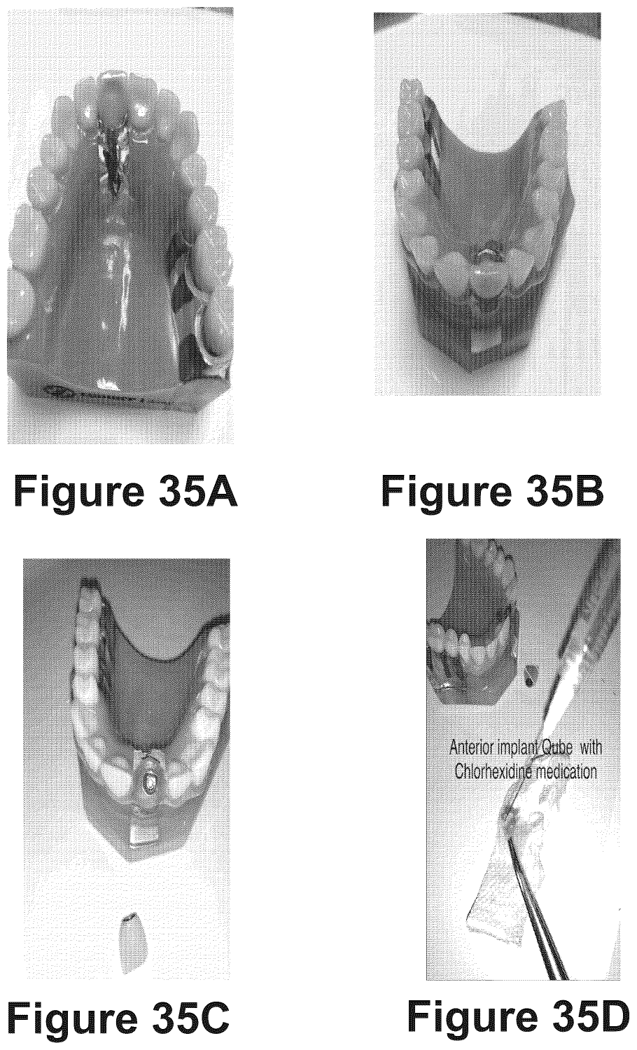

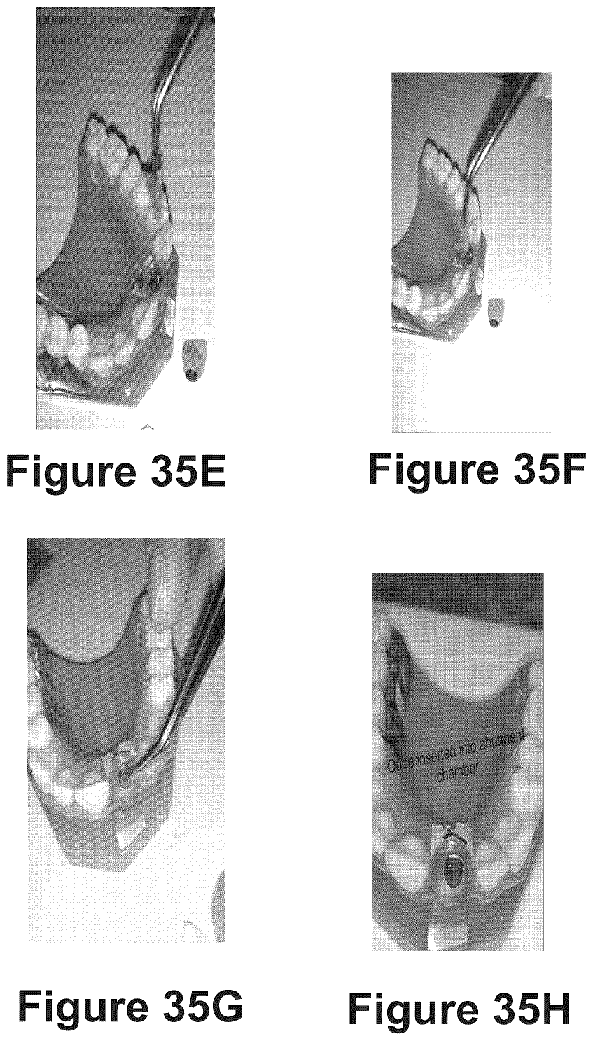

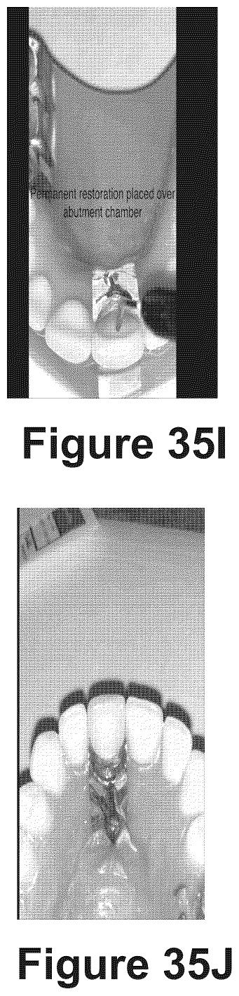

FIGS. 35A-35J illustrate an oral surgical process involving use of an anterior implant qube. In an example embodiment, the anterior implant qube may carry a medicament such as chlorhexidine. The anterior implant qube is inserted into a chamber in an abutment coupled to an anterior dental implant. A permanent restoration is subsequently placed over the abutment chamber.

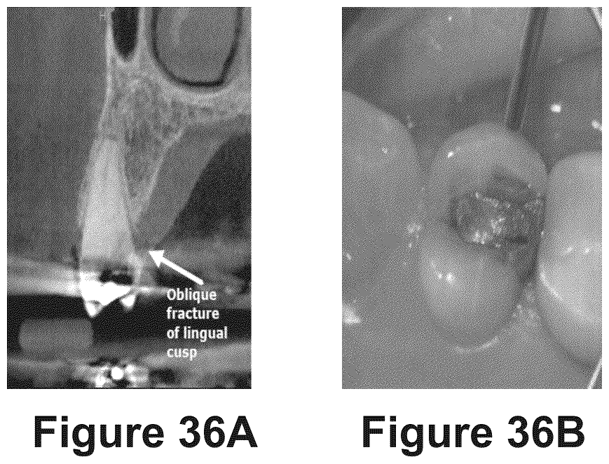

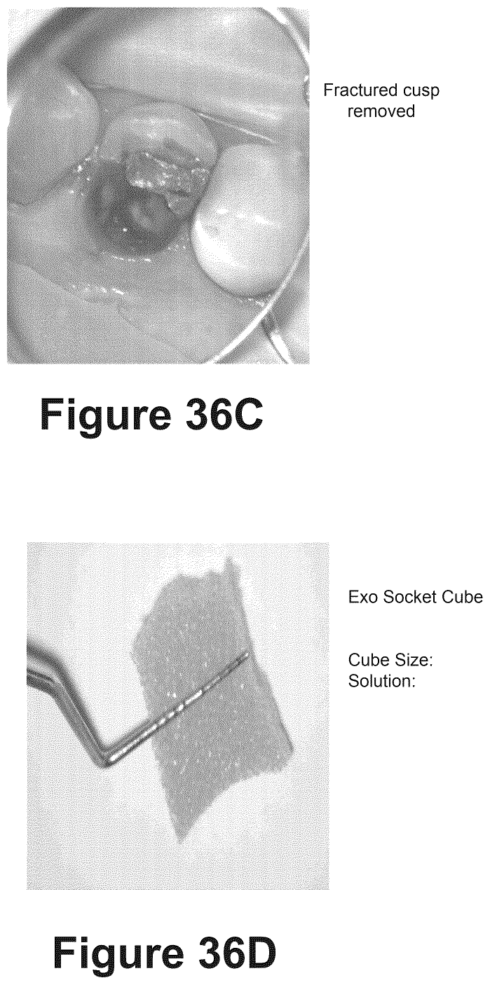

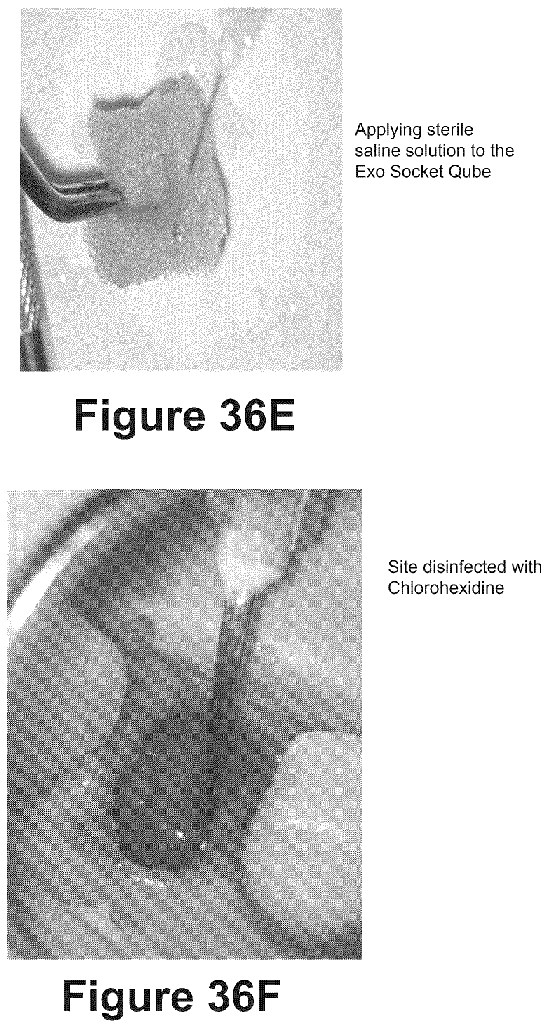

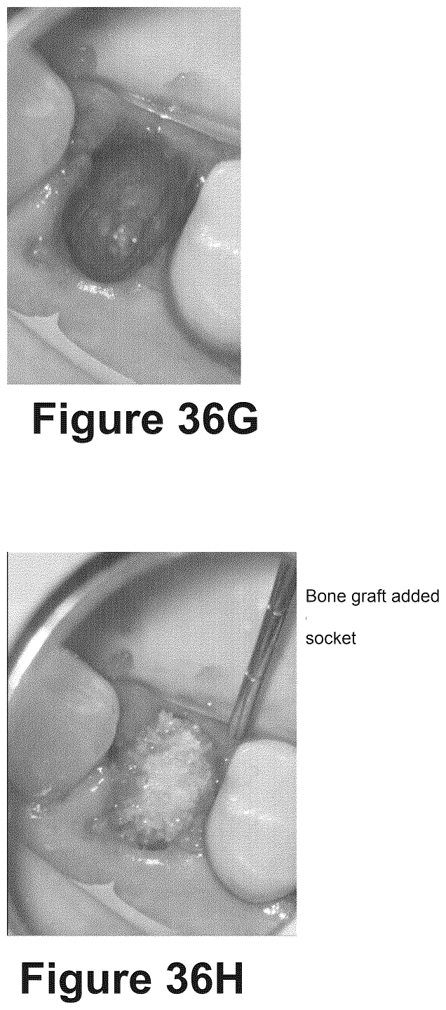

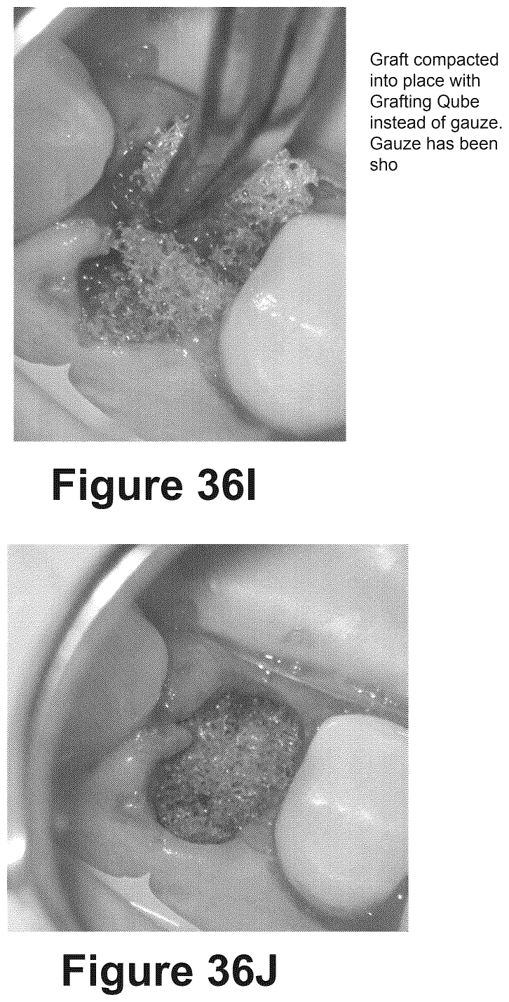

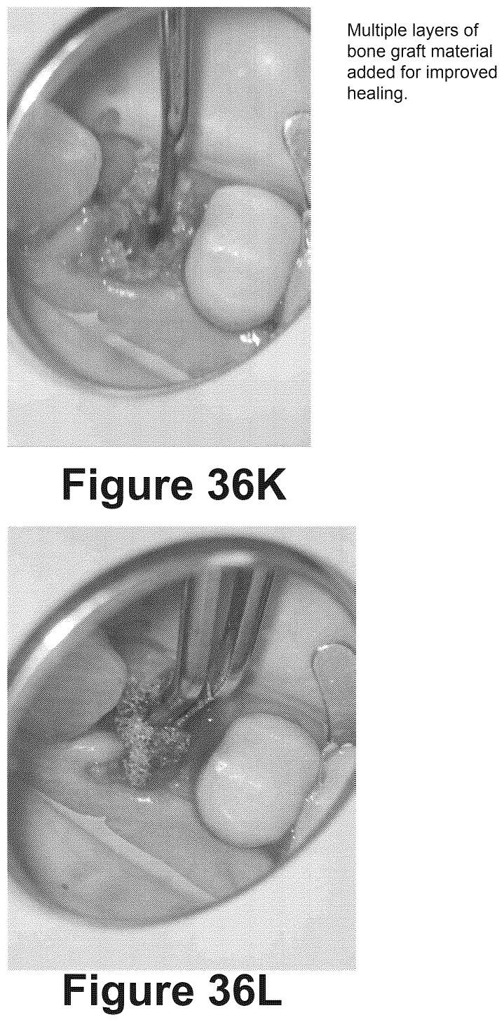

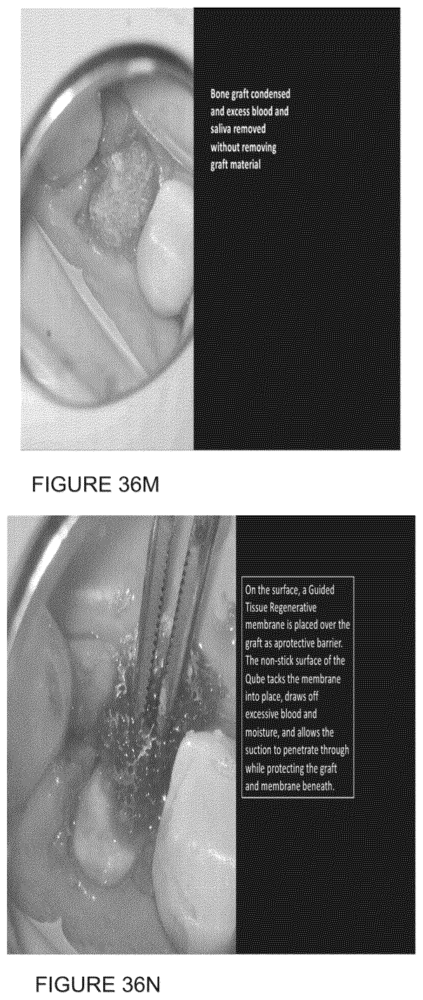

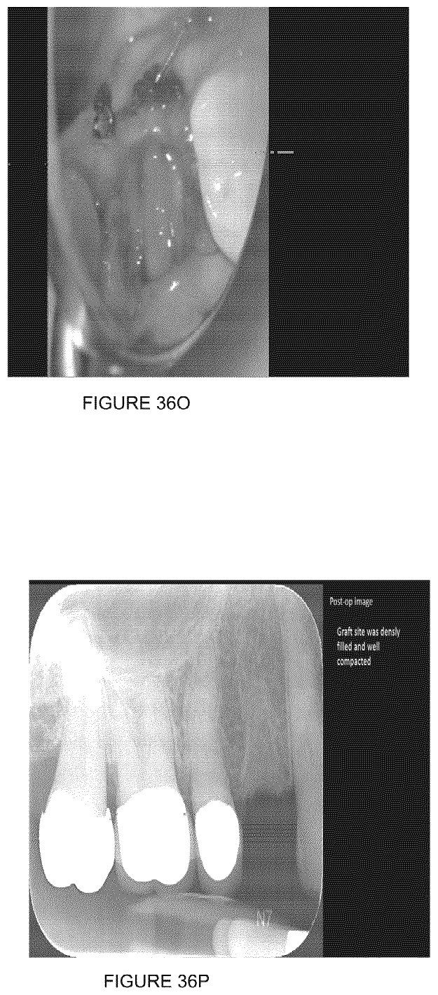

FIGS. 36A-36P illustrate an oral surgical process involving use of both an exo qube and a grafting qube.

FIG. 36B is an x-ray showing an oblique fracture of a lingual cusp.

FIG. 36C shows another step in the oral surgical process.

FIG. 36D shows that the fractured cusp has been removed.

FIG. 36E shows an example embodiment of an exo socket qube.

FIG. 36F shows the exo socket qube of FIG. 36E with a sterile saline solution being applied to it.

FIG. 36G shows the tooth extraction site being disinfected with chlorohexidine.

FIG. 36H shows the disinfected tooth extraction site.

FIG. 36I shows bone graft material that has been added over and into the tooth socket recess from where the extracted tooth has been removed.

FIG. 36J shows the graft material being compacted into place by using a grafting qube to apply pressure to the grafting material.

FIG. 36K shows the grafting qube over the graft material at the extraction socket recess site.

FIG. 36L shows that multiple layers of bone graft material are being added for improved healing in accordance with example embodiments.

FIGS. 36M-36N illustrate condensed bone graft with excess blood and saliva have been removed without removing graft material in accordance with example embodiments.

FIG. 36O illustrates a step of placing a guided tissue regenerative or GTR membrane over the graft as a protective barrier. The non-stick surface of the qube tacks the membrane into place, draws off excessive blood and moisture, and allows the suction to penetrate through while protecting the graft and membrane beneath.

FIGS. 36P-36Q show a graft site that has been densely filled and well compacted.

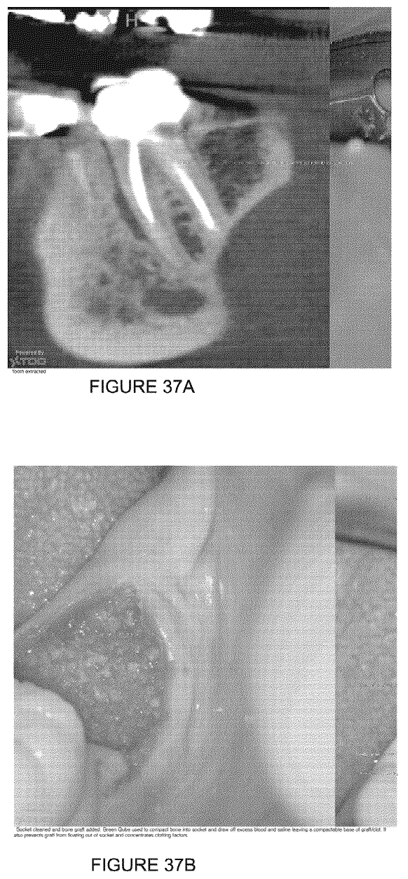

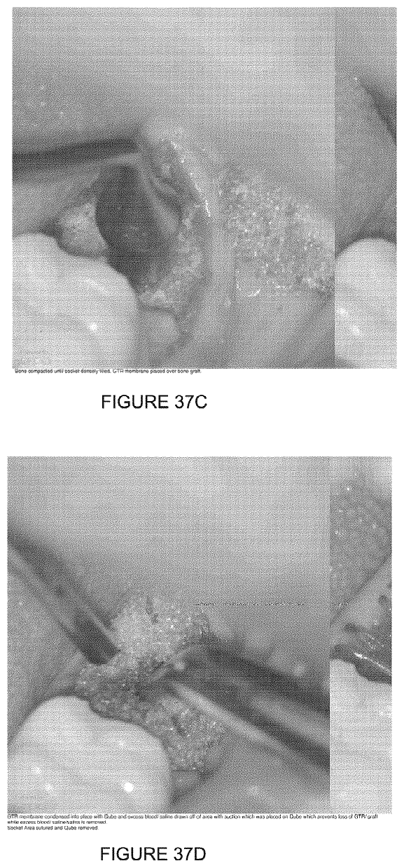

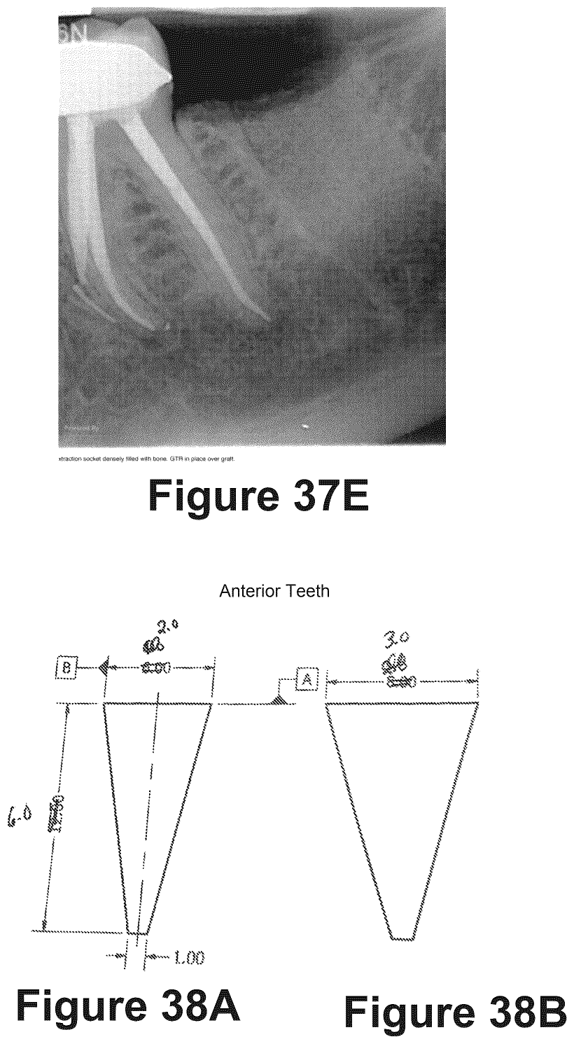

FIGS. 37A-37E illustrate another oral surgical process involving use of a graft qube.

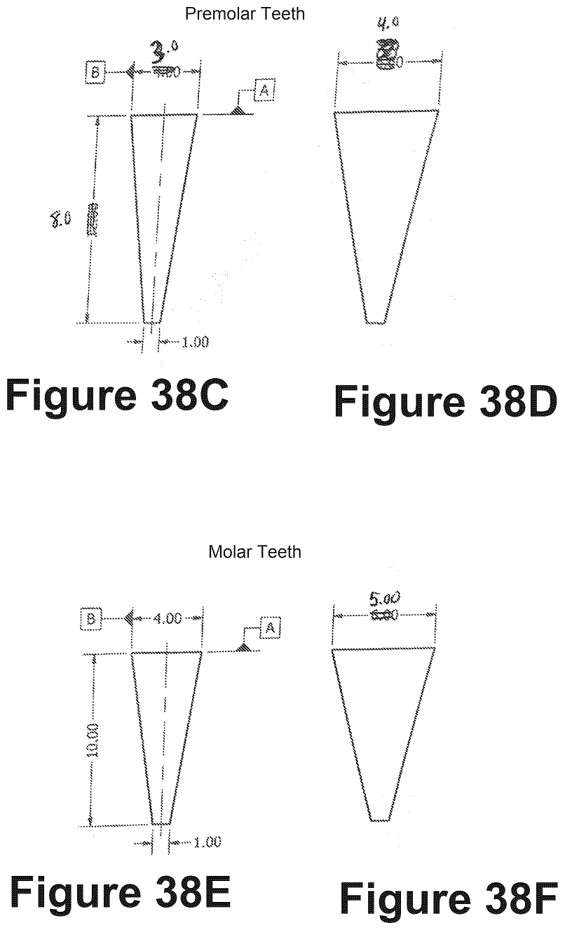

FIGS. 38A-38F illustrate qubes for use with oral surgical processes involving anterior teeth, premolar teeth, and molar teeth.

FIGS. 38A-38B illustrate qubes for use with oral surgical processes involving anterior teeth.

FIGS. 38C-38D illustrate qubes for use with oral surgical processes involving premolar teeth.

FIGS. 38E-38F illustrate qubes for use with oral surgical processes involving molar teeth.

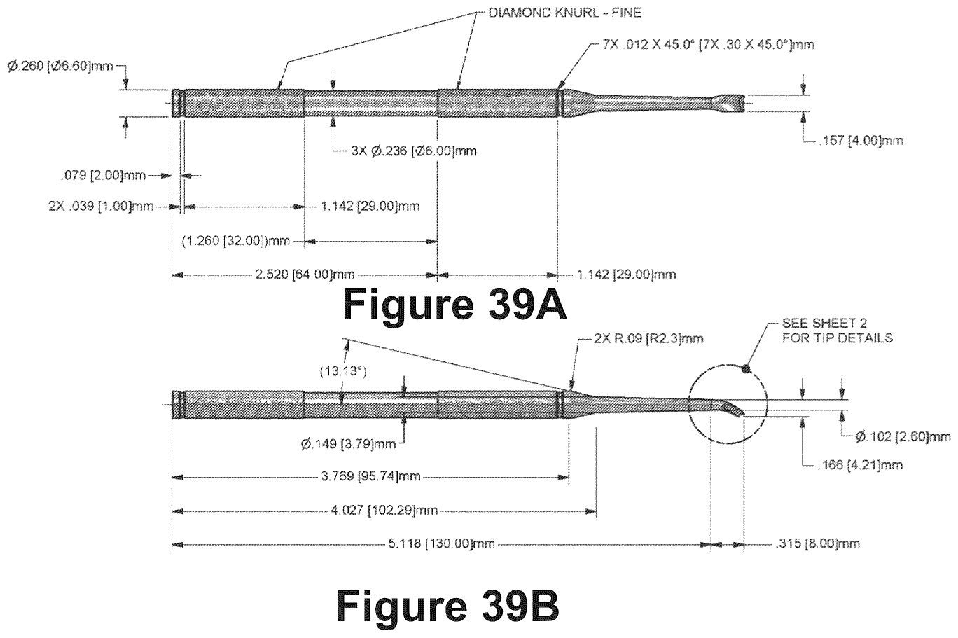

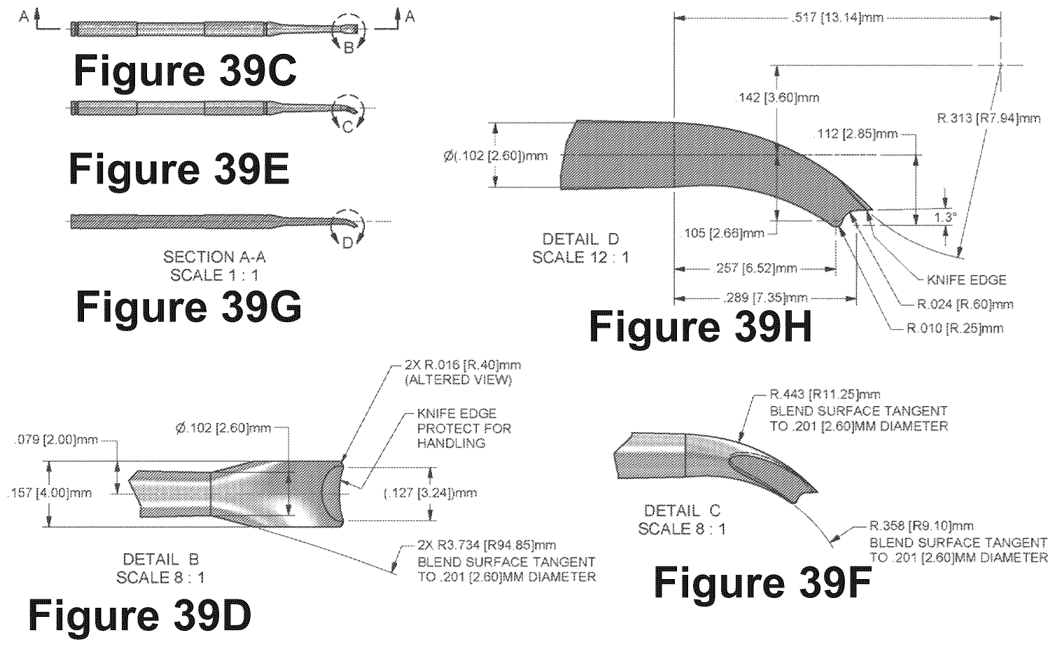

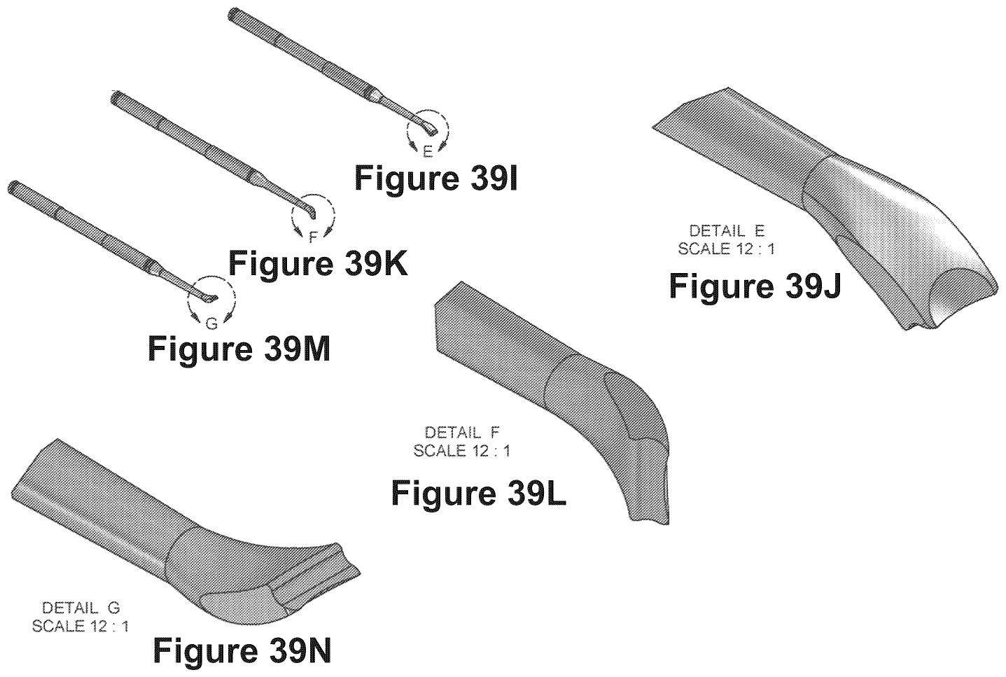

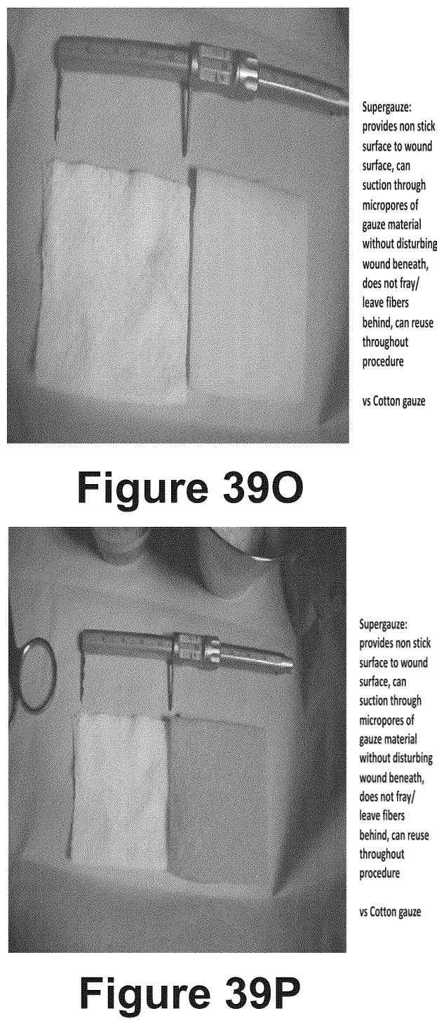

FIGS. 39A-39N illustrate retraction tools in accordance with example embodiments.

FIGS. 39O-39P illustrate SG series qubes as substitutes for cotton gauze in accordance with example embodiments.

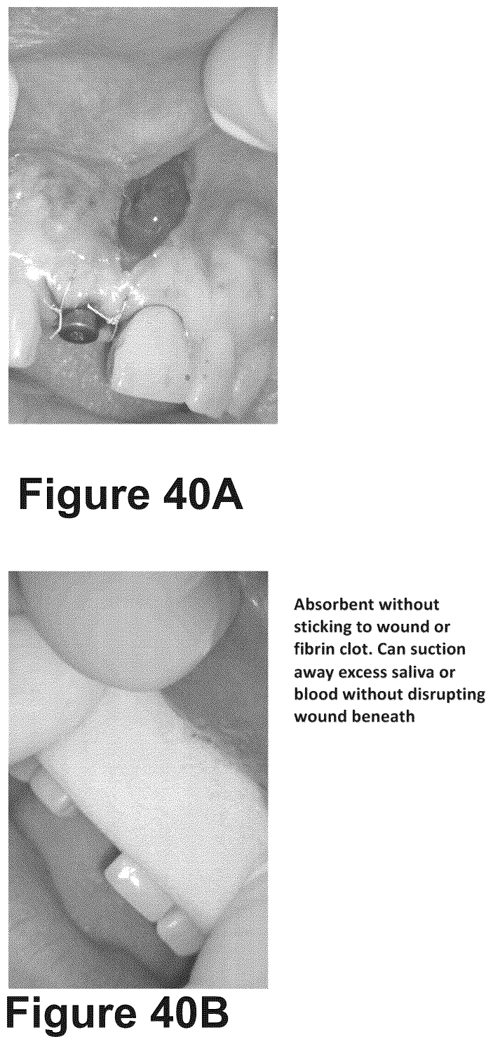

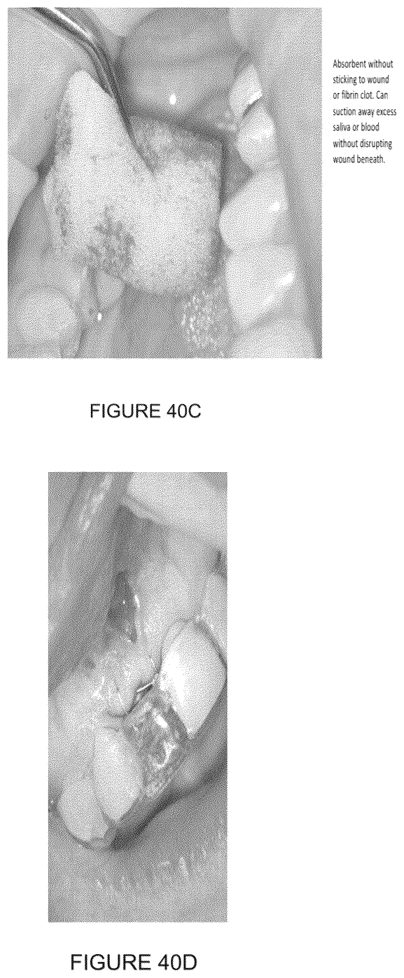

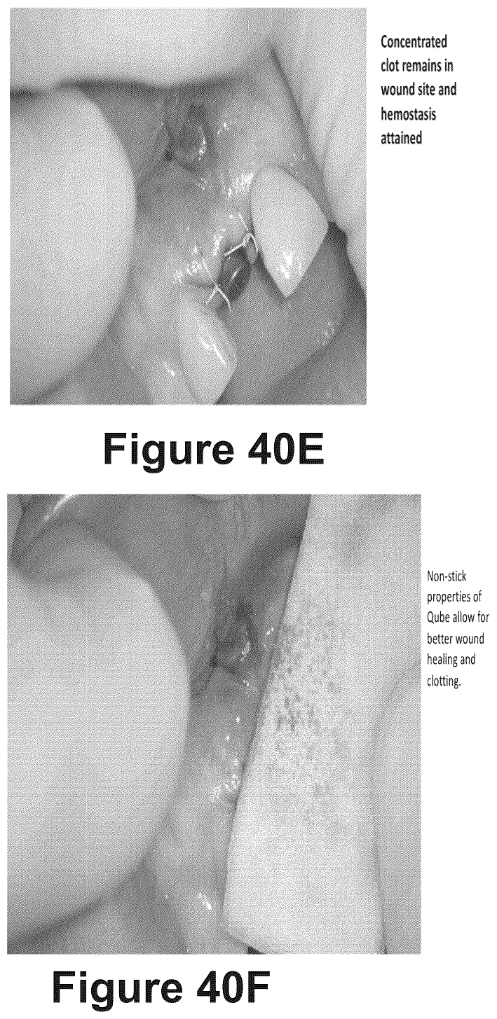

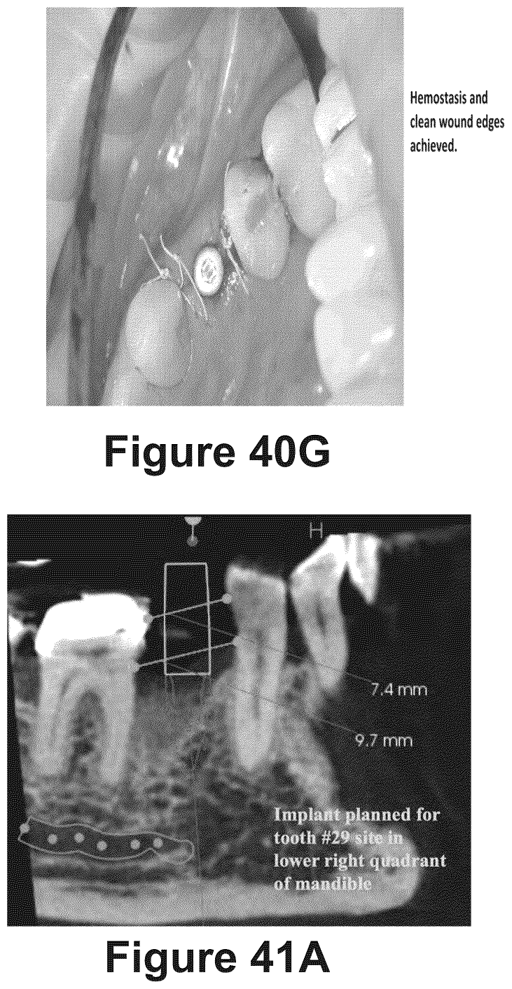

FIGS. 40A-40G illustrate oral surgical process steps involving use of qubes for enhancing hemostasis, wound cleaning, wound healing, excess saliva and blood absorption and suctioning.

FIGS. 40B-40C illustrate how qubes which are configured in accordance with example embodiments may be absorbent without sticking to a wound or fibrin clot. Use of qubes enables suctioning of excess saliva and blood without disrupting a wound beneath.

FIGS. 40D-40E show that a concentrated clot remains in the wound site and hemostasis has been attained.

FIG. 40F shows how the non-stick properties of qubes allow for better wound healing and clotting.

FIG. 40G shows that hemostasis and clean wound edges have been achieved.

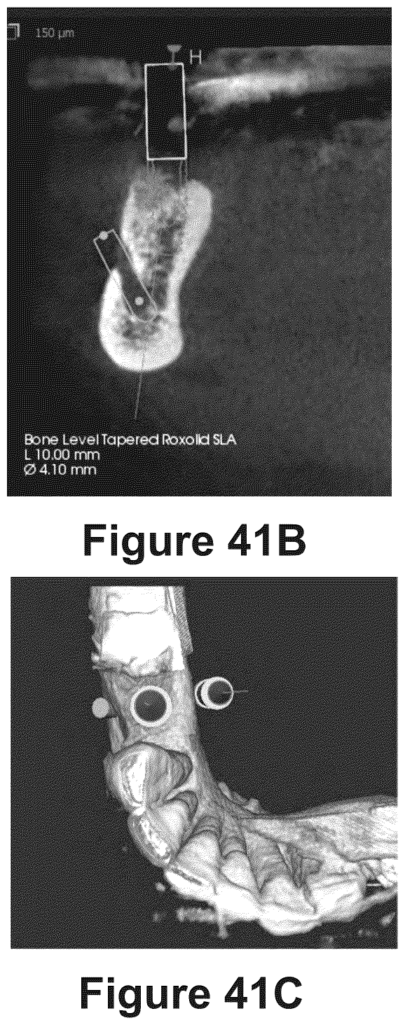

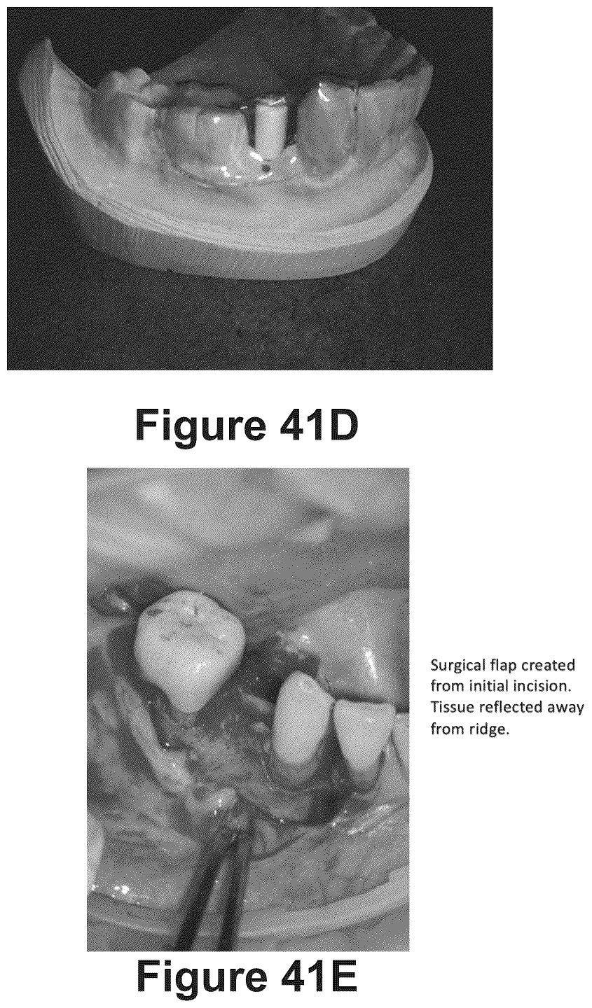

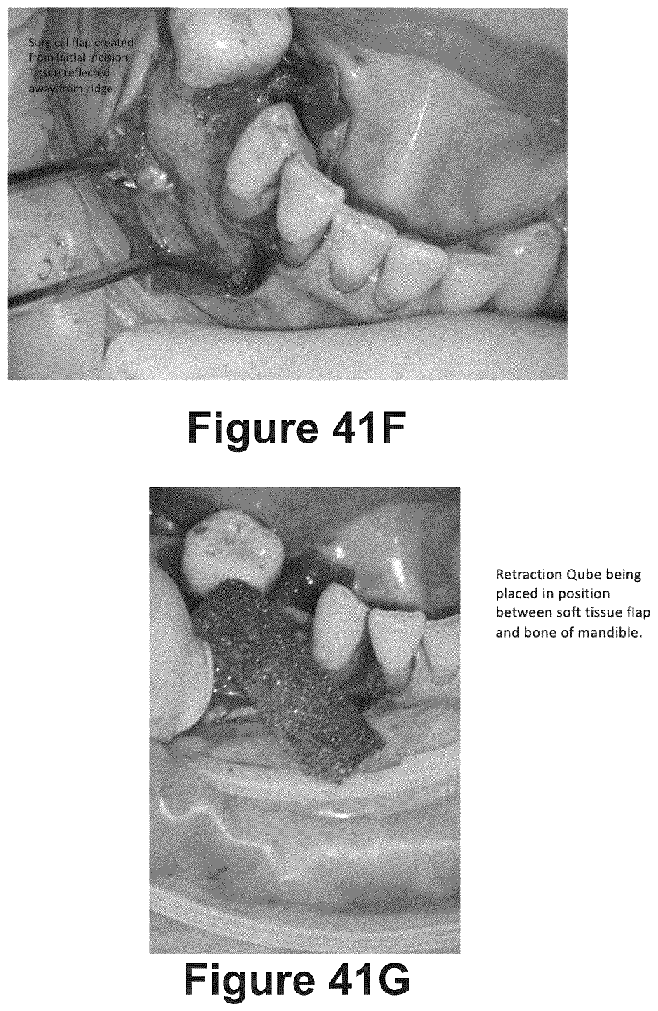

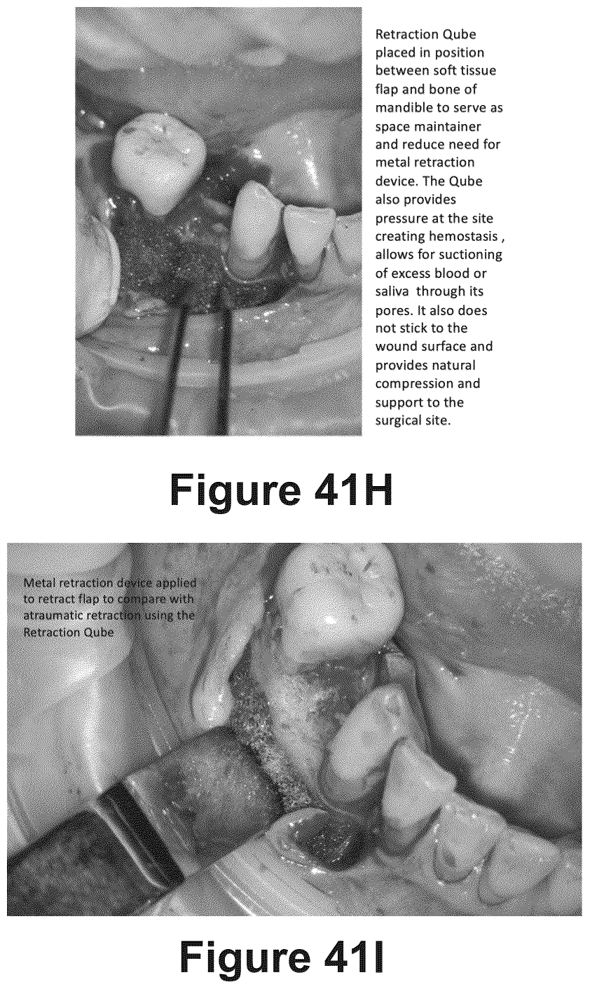

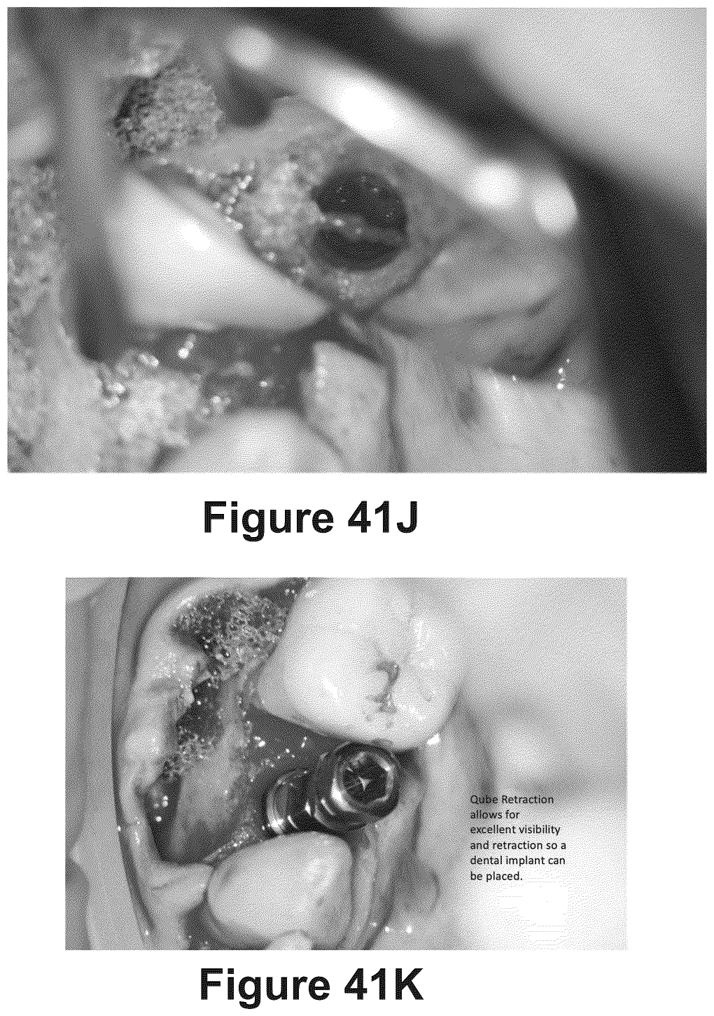

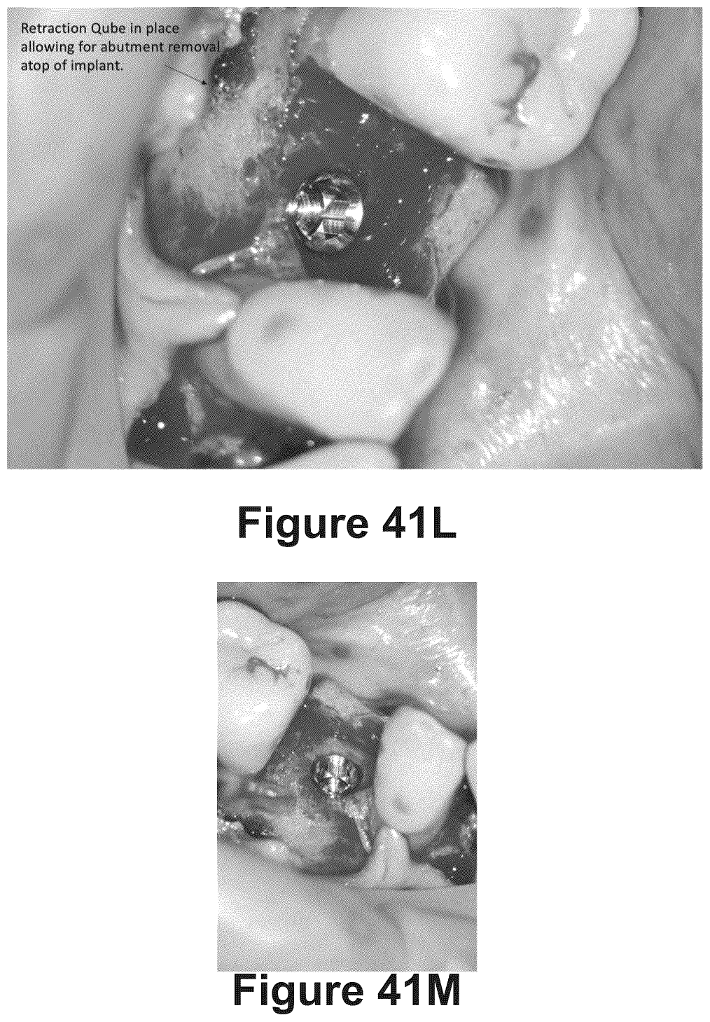

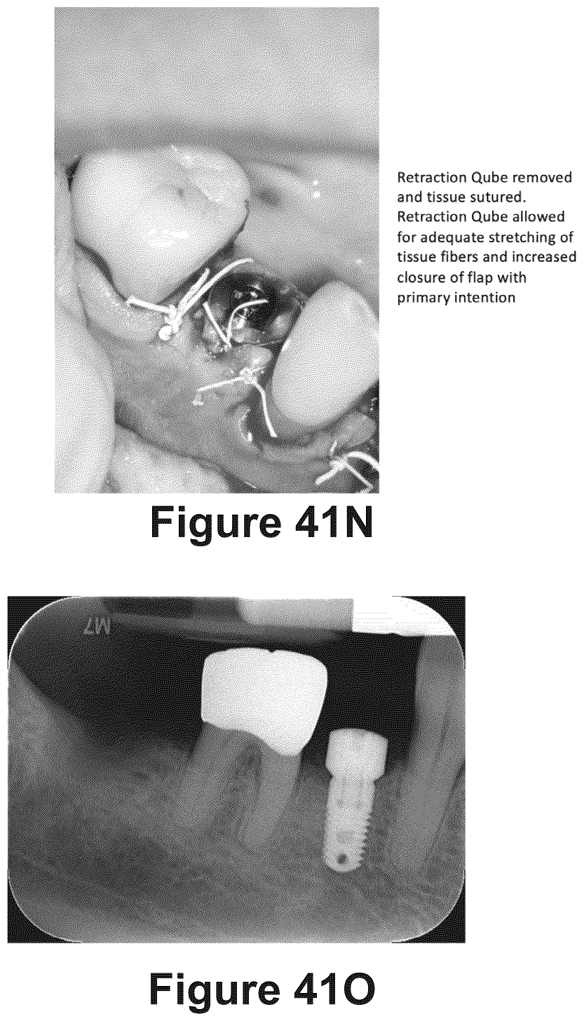

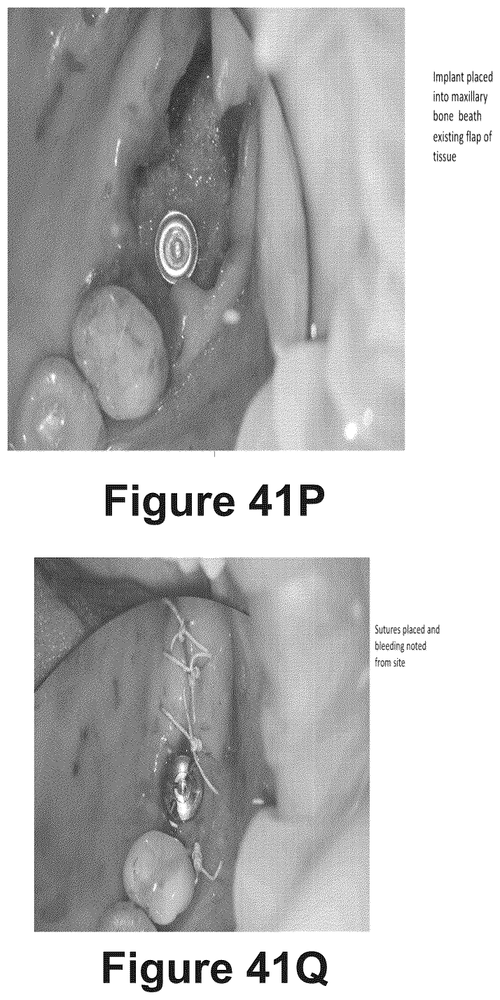

FIGS. 41A-41Q illustrate one or more dental implant processes involving use of one or more retraction qubes.

FIGS. 41A-41D shows that an implant is planned for a tooth site in a lower right quadrant of the mandible.

FIGS. 41E-41F show surgical flaps that have been created from initial incisions in example embodiments. Tissue is reflected away from the ridge.

FIG. 41G shows a retraction qube being placed in position between a soft tissue flap and a bone of mandible.

FIG. 41H shows a retraction qube placed in a position between a soft tissue flap and a bone of mandible to serve as a space maintainer and to reduce a need for a metal retraction device. The qube also provides pressure at the site, creating hemostasis and allowing for suctioning of excess blood and saliva through pores within the qube. The qube also does not stick to the wound surface and provides natural compression and support to the surgical site.

FIG. 41I shows a metal retraction device applied to retract a flap to compare with the atraumatic retraction provided by a retraction qube.

FIGS. 41J-41K show that qube retraction allows for excellent visibility and retraction so a dental implant can be placed.

FIG. 41L shows a retraction qube in place allowing for abutment removal atop an implant.

FIG. 41M-41N show an implant site after a retraction qube has been removed and tissue sutured. The retraction qube allowed for adequate stretching of tissue fibers and increased closure of a flap with primary intention.

FIGS. 41O-41P show an implant placed into a maxillary bone beneath an existing flap of tissue.

FIG. 41Q shows sutures placed with very little bleeding noted from the site of the tooth extraction and dental implantation.

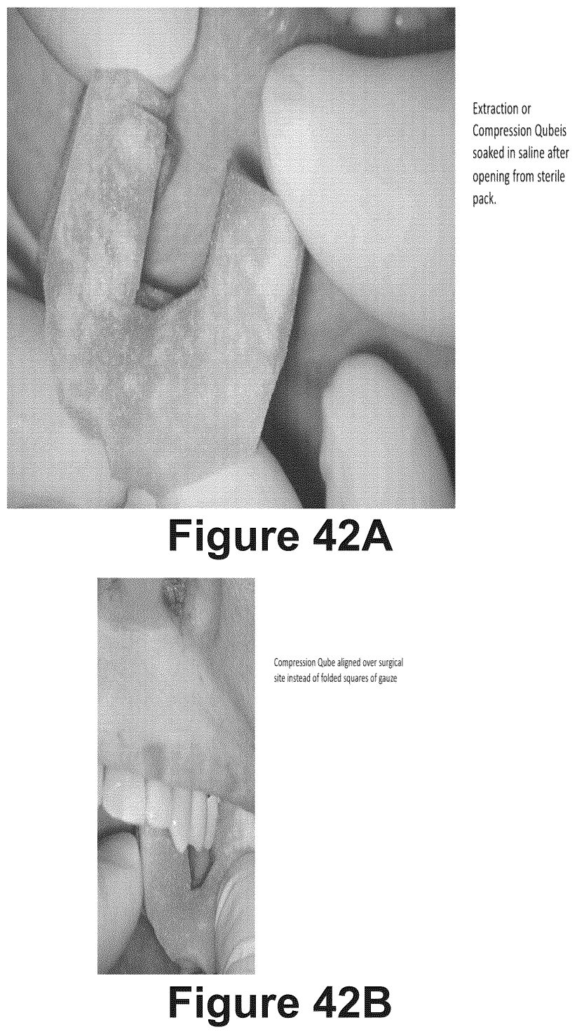

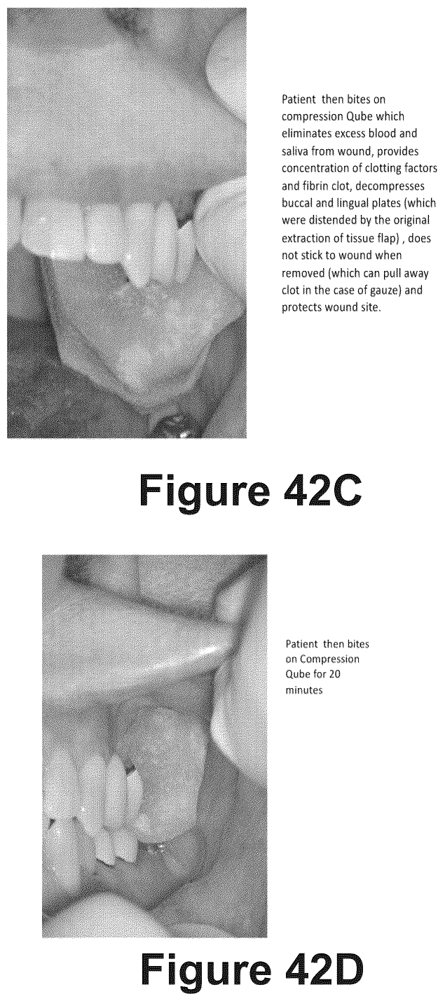

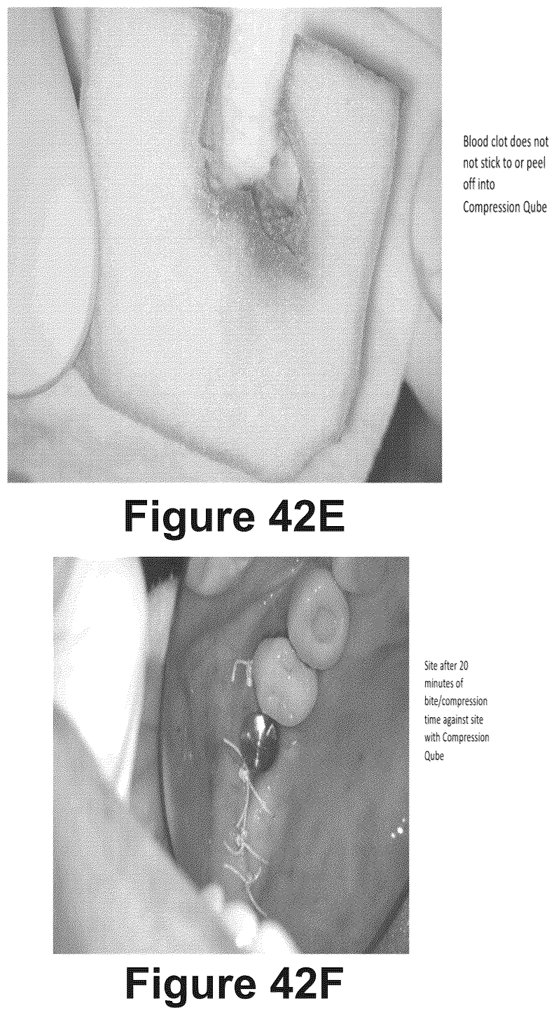

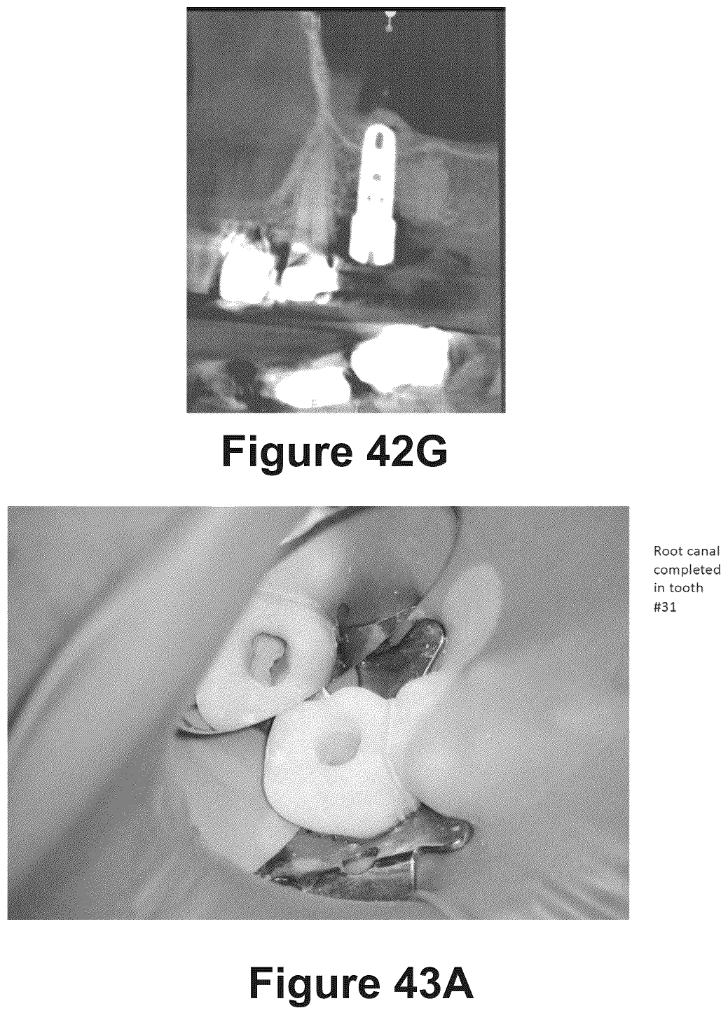

FIGS. 42A-42G illustrate one or more tooth extraction and/or dental implant processes involving use a compression qubes or C series qubes or C qubes in accordance with example embodiments.

FIG. 42A shows an extraction or compression qube that is soaked in saline after opening from a sterile pack.

FIG. 42B illustrates a compression qube aligned over a surgical site instead of a conventional folded square of cotton gauze.

FIG. 42C shows a patient biting on a compression qube which eliminates excess blood and saliva from a tooth extraction wound and provides concentration of clotting factors and a fibrin clot. Buccal and lingual plates are decompressed which were distended by the original extraction of a tissue flap. The compression qube protects the wound site and does not stick to the wound when removed in contrast to cotton gauze having a tendency to pull away a clot.

FIG. 42D shows a patient biting on a compression qube. The patient typically will do so for 20 minutes or so.

FIG. 42E shows that a blood clot does not stick to nor peel off into a compression qube in accordance with example embodiments.

FIG. 42F shows a dental implant site after 20 minutes of bite compression time against the site with a compression qube in accordance with example embodiments.

FIG. 42G shows an x-ray of the dental implant from a side view.

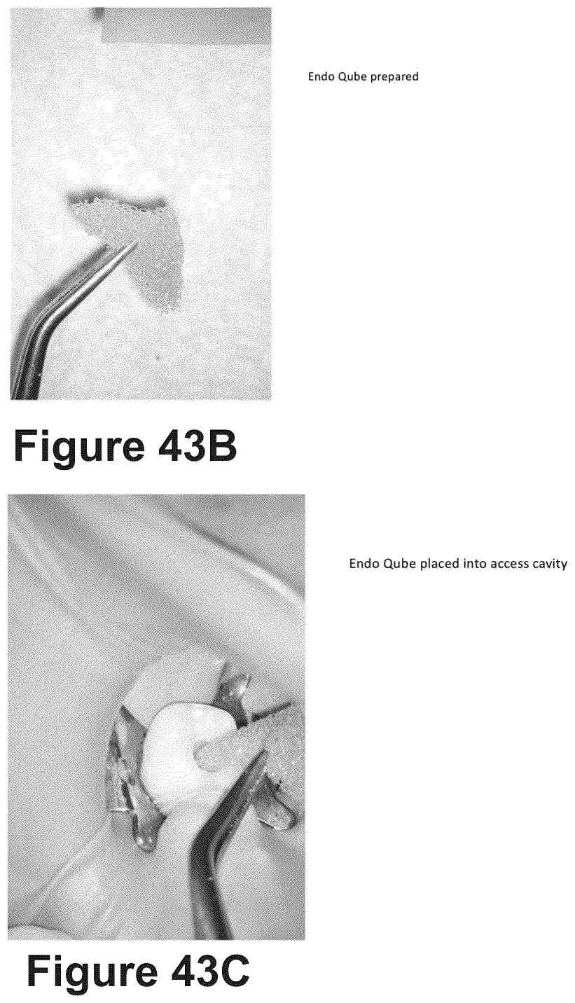

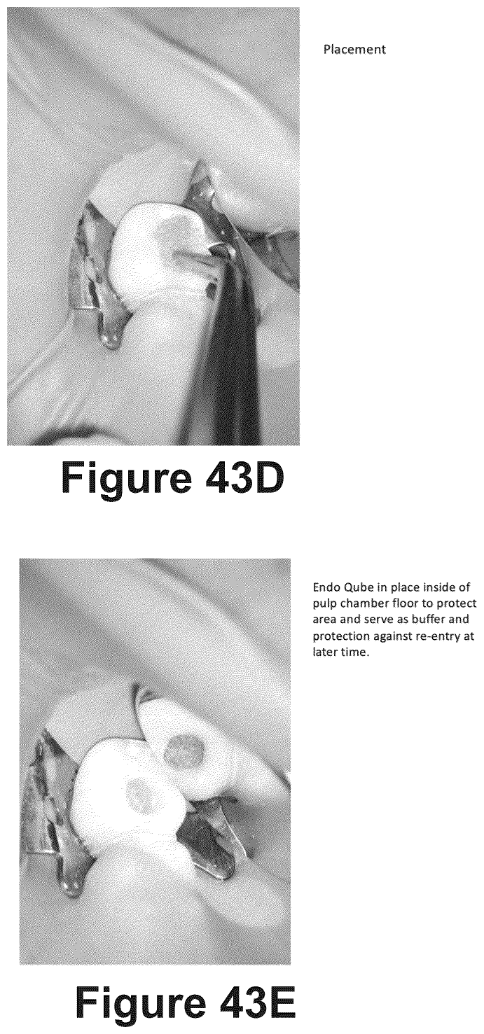

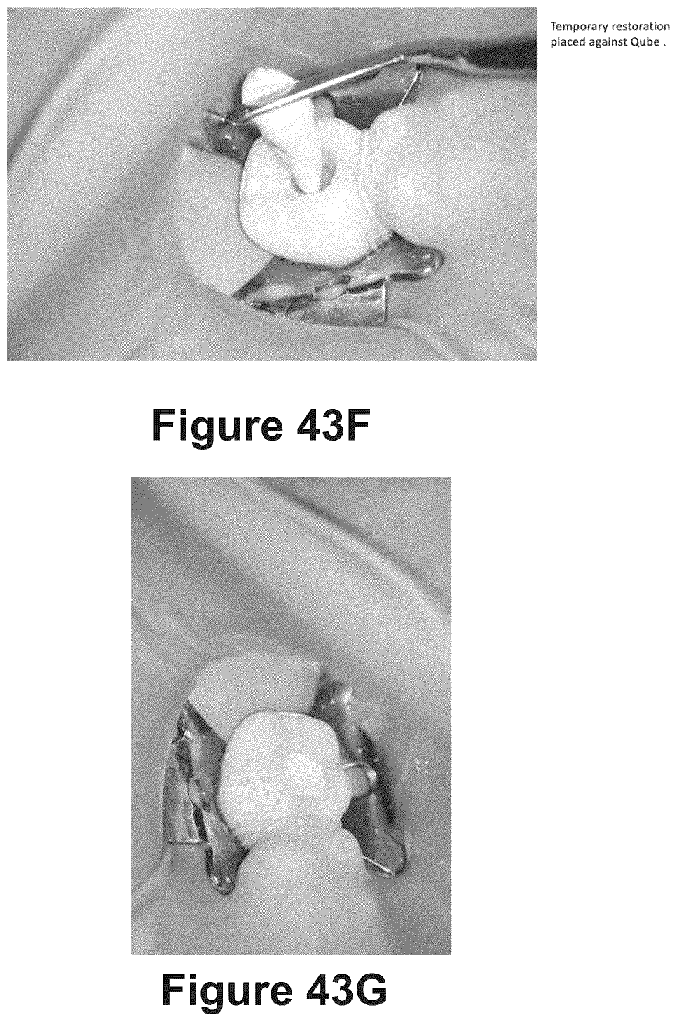

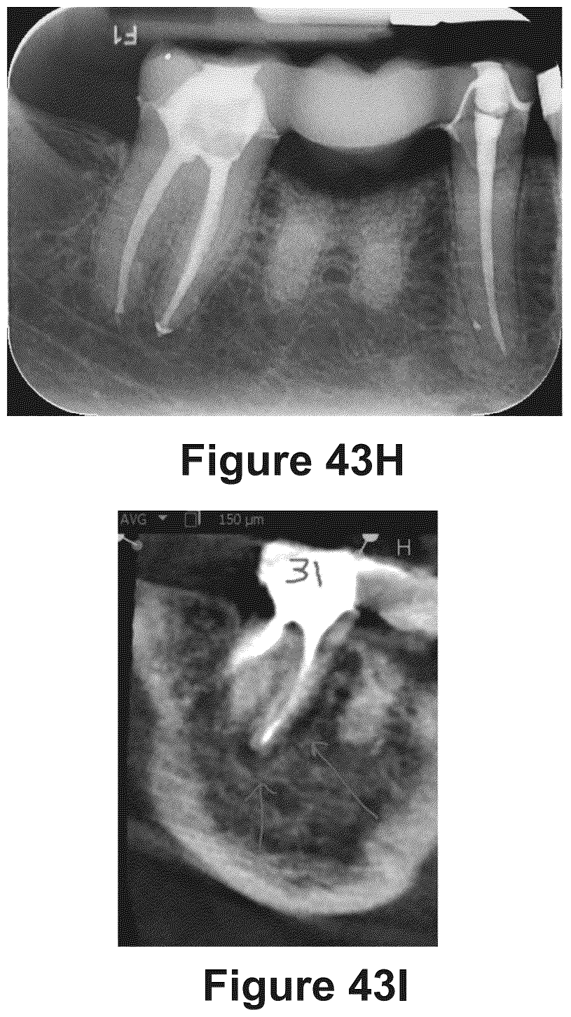

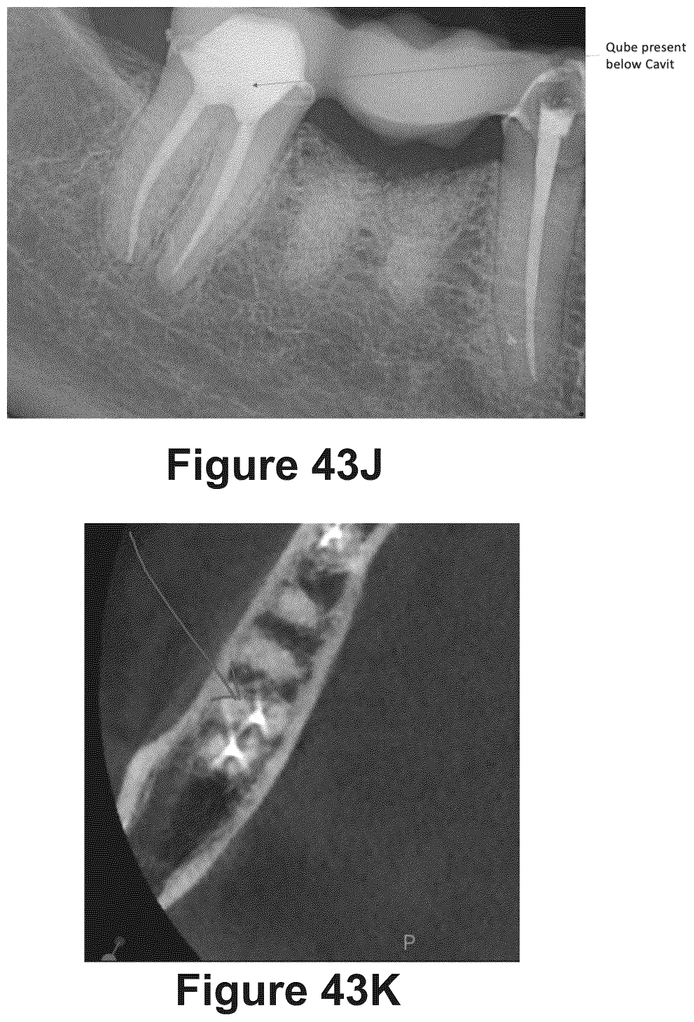

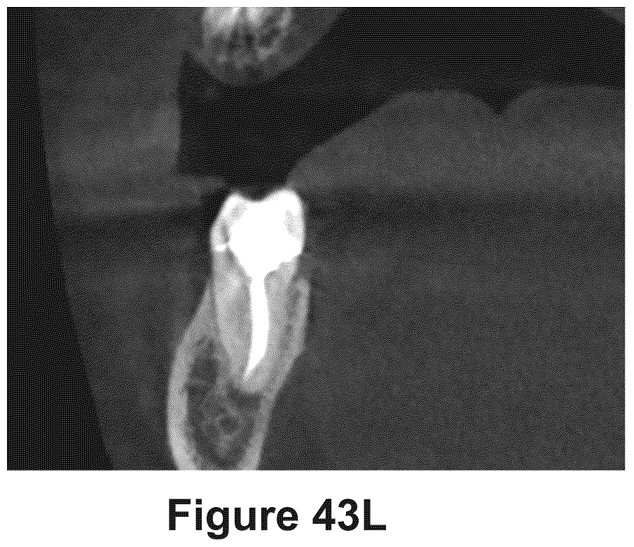

FIG. 43A-43L illustrate one or more oral surgical processes involving use of endo qubes in accordance with example embodiments.

FIG. 43A shows a completed root canal.

FIG. 43B shows a prepared endo qube in accordance with an example embodiment.

FIG. 43C shows an endo qube being placed into an access cavity of a tooth in an example embodiment.

FIG. 43D shows the placement of the endo qube into the access cavity of the tooth.

FIG. 43E shows an endo qube in place inside a pulp chamber floor to protect the area and provide a buffer and protection against re-entry at a later time.

FIG. 43F shows a temporary restoration being placed against the endo qube in an example embodiment.

FIG. 43G-43L show the presence of the endo qube below the temporary restoration within the cavity.

While the invention has been described in terms of several example embodiments, those skilled in the art will recognize that the invention can be practiced with modification and alteration. The description is thus to be regarded as illustrative.

For example, each of the SG, C, G, MS and R series qubes, and dab qubes, applicator qubes, qube rolls may be available in any or all of broad ranges of sizes, shapes and/or proportions similar to the examples described with reference to the R series qube or otherwise. As another example, further oral surgical or non-surgical dental applications of qubes may include root canal surgeries, teeth straightening, and teeth whitening. Example embodiments of qubes may be formed with ePTFE, PTFE, polyester polyurethane or polyether polyurethane in this and other contexts or with different materials such as those identified above herein or others with one or more similar or equivalent properties or characteristics.

In another example, a parotid qube may be configured to block saliva flow from one of two parotid gland areas that are located in the mouth just below and in front of each ear. The parotid qube may be elongated rhombohedral, trapezoidal or rectangular or tubular in shape in some examples. A parotid qube may be double or triple felted or be configured or manufactured with 1.5×, 2×, 2.5×, 3× or up to five times (5×) compression.

There are also many practical applications in addition to dentistry wherein conventional use of cotton gauze may be advantageously replaced with the use of qubes as set forth herein or as may be modified to suit those particular uses. Such applications may include sutures, sponges, retractors and other items used in certain medical bodily surgeries other than oral surgeries, and first aid items such as bandages and wraps, and swabs, medicinal or cosmetic applicators, sponges and bodily fluid filters. The material forming a qube in alternative embodiments may include polyurethane additives such as hydroperoxide, bronze powder, isothiazolinone, zinc pyrithinone, thiabendazole, silver, quaternary ammonium, 10,10′oxybisphenox-arsine (OBPA), silicon oil, silicone surfactant, polyether, polyester, polyethyl, polyvinyl alcohol, or polydibutyltitanate. Hydroxyl values may be varied, e.g., between 27-58 mg/g. Acid values may be varied, e.g., between 0.05-0.08 mg/g. Water values may be varied, e.g., between 0.01-0.10%. Viscosity values may be varied, e.g., between 400-1225. Qube shapes can be modified in example embodiments by rounding corners that form right angles or otherwise smoothing sharp edges and/or corners.

A kit may include one or more of multiple types of qubes such as two of more of SG, C, G, MS and R series qubes may be included in the kit, and all five of these series types may include one or two or three or several qubes, and Qubes configured for use in applications and procedures other than these five that have been described herein may be included in kits with other series type Qubes and/or within kits of their own. The qubes of each series type may be provided in different colors to easily distinguish them and/or may be provided in drawers or containment sections of the kit that are separated and labelled by series type and/or by specific applications or uses.

In addition, in methods that may be performed according to embodiments described herein and that may have been described above, the operations have been described in selected typographical sequences. However, the sequences have been selected and so ordered for typographical convenience and are not intended to imply any particular order for performing the operations, except for those where a particular order may be expressly set forth or where those of ordinary skill in the art may deem a particular order to be necessary.

A group of items linked with the conjunction “and” in the above specification should not be read as requiring that each and every one of those items be present in the grouping in accordance with all embodiments of that grouping, as various embodiments will have one or more of those elements replaced with one or more others. Furthermore, although items, elements or components of the invention may be described or claimed in the singular, the plural is contemplated to be within the scope thereof unless limitation to the singular is explicitly stated or clearly understood as necessary by those of ordinary skill in the art.

The presence of broadening words and phrases such as “one or more,” “at least,” “but not limited to” or other such phrases in some instances shall not be read to mean that the narrower case is intended or required in instances where such broadening phrases may be absent.

1. An oral surgical retraction article, comprising a polyester or polyether polyurethane foam sponge, or a combination thereof, that is autoclavable at 250° F. and has a porosity between not less than 45 pores per inch (ppi) and not more than 105 ppi and configured in size and shape for retracting a gingival flap during an oral surgery.

2. The oral surgical retraction article of claim 1, wherein said polyester or polyether polyurethane foam sponge includes an elongated shape.

3. The oral surgical retraction article of claim 2, wherein said polyester or polyether polyurethane foam sponge includes a cylindrical, ellipsoidal, tubular, wedge, prism, ovoid, triovoid, egg or pear shape, or combinations thereof.

4. A dental implant surgical spacer article, comprising a polyester or polyether polyurethane foam sponge, or combinations thereof, that is autoclavable at 250° F. and has a porosity between not less than 45 pores per inch (ppi) and not more than 105 ppi and configured in size and shape to preserve a volume above a dental implant for coupling an abutment to the dental implant during an osseointegration period.

5. The dental implant surgical spacer article of claim 4, wherein the polyester or polyether polyurethane foam sponge comprises a base end opposite a tapered end.

6. The dental implant surgical spacer article of claim 4, wherein the polyester or polyether polyurethane foam sponge comprises a tapered end to base end weight density ratio of at least 2:1.

7. The dental implant surgical spacer article of claim 4, wherein the polyester or polyether polyurethane foam sponge comprises a conic or truncated conic shape.

8. The dental implant surgical spacer article of claim 4, wherein the polyester or polyether polyurethane foam sponge comprises a pyramid or truncated pyramid shape.

9-16. (canceled)

17. A dental surgical spacer method, comprising placing a polyester or polyether polyurethane foam sponge, as recited at claim 4, in a space next to an embedded dental implant to preserve a spacing for coupling an abutment to the dental implant after an osseointegration period.

18. An oral surgical retraction method, comprising placing a polyester or polyether polyurethane foam sponge, as recited at claim 1, and thereby retracting said gingival flap during said oral surgery.

19. (canceled)

20. A dental article, comprising a polyester or polyether polyurethane foam sponge, or combinations thereof, that is autoclavable at 250° F. and has a porosity between not less than 45 pores per inch (ppi) and not more than 105 ppi, and that is configured for nonadhesively contacting and compressing loose graft material contained within a bone socket recess.

21. The dental article of claim 20, wherein said sponge comprises a 2D or 3D mushroom shape.

22. The dental article of claim 20, wherein said sponge comprises a 2D or 3D football shape.

23. The dental article of claim 20, wherein the polyester or polyether polyurethane foam sponge, or a second polyester or polyether polyurethane foam sponge, is configured for maintaining a volume density integrity of compressed graft material when removing bodily fluids from the bone socket recess by suctioning said fluids through the sponge.

24. The dental article of claim 23, wherein said second sponge comprises a U shape.

25. The dental article of claim 20, further comprising a guided tissue regeneration membrane configured to be disposed over the compressed graft material following said suctioning.

26. The dental article of claim 25, wherein said membrane is configured to remain over said graft material within said socket graft recess during an osseointegration period.

27. A dental bone socket grafting method, comprising:
preparing a bone socket recess defined within a patient's jawbone;
after said preparing said bone socket recess, filling the bone socket recess with loose graft material; and
compressing the loose graft material within the bone socket recess by inserting a nonadhesive polyester or polyether polyurethane foam sponge, or combinations thereof, as recited at claim 20, into contact with the loose graft material therein and applying pressure to said sponge.

28. The dental bone socket grafting method of claim 27, wherein the preparing a bone socket recess comprises shape cutting or drilling into a tooth, or through gum tissue, or into some bone tissue, or combinations thereof.

29. (canceled)

30. The dental bone socket grafting method of claim 27, comprising suctioning fluid from the bone socket recess through said nonadhesive polyester or polyether polyurethane foam sponge.
